(19) United States
(12) Patent Application Publication
(10) Pub. No.: US 2025/0361296 A1
(43) Pub. Date: Nov. 27, 2025

(54) ANTI-SIGLEC15 ANTIBODIES AND USES THEREOF

(71) Applicants: Biocytogen Pharmaceuticals (Beijing) Co., Ltd., Beijing (CN); Shanghai Institute of Biological Products Co., Ltd., Shanghai (CN)

(72) Inventors: Yongfei Yang, Beijing (CN); Fengping Yao, Beijing (CN); Pan Song, Beijing (CN); Aidong Qu, Shanghai (CN); Xiuling Li, Shanghai (CN); Hongyuan Liang, Shanghai (CN)

(21) Appl. No.: 18/873,872

(22) PCT Filed: Jun. 13, 2023
(86) PCT No.: PCT/CN2023/099828

§ 371 Date: Dec. 11, 2024

(30) Foreign Application Priority Data
Jun. 13, 2022 (WO) PCT/CN2022/098378
Nov. 25, 2022 (WO) PCT/CN2022/134289

(51) Int. Cl. C07K 16/28; A61K 39/00; A61K 47/68; A61P 35/00; C12N 15/85

(52) U.S. Cl. CPC C07K 16/2803; A61K 47/6849; A61P 35/00; C07K 16/2818; C07K 16/2827; C07K 16/2878; C12N 15/85; A61K 2039/507; C07K 2317/14; C07K 2317/24; C07K 2317/565; C07K 2317/71; C07K 2317/76; C12N 2800/40

(57) ABSTRACT

This disclosure provides an anti-SIGLEC15 antibody, antigen-binding fragment, and the use thereof.

### CLAIM OF PRIORITY

This application claims the benefit of PCT Application No. PCT/CN2022/098378, filed on Jun. 13, 2022, and PCT Application No. PCT/CN2022/134289, filed on Nov. 25, 2022. The entire contents of the foregoing are incorporated herein by reference.

### TECHNICAL FIELD

This disclosure relates to anti-SIGLEC15 (Sialic acid binding Ig-like lectin 15) antibodies and uses thereof.

### BACKGROUND

SIGLEC15 (Sialic acid-binding immunoglobulin-like lectin 15) belongs to the SIGLEC family and is a type I transmembrane protein. It is rarely expressed in most normal human tissues and immune cell subsets, but has relatively high expression in macrophages. In 2007, Japanese scientist Takashi Angata first discovered that this protein can recognize sialic acid, so it was classified as the SIGLEC family. By regulating innate and adaptive immune responses, SIGLEC15 plays an important role in autoimmune diseases, inflammatory responses and tumors. Considering the important role of SIGLEC15 in autoimmune diseases, inflammatory responses and tumors, there is a need to develop a therapeutic agent targeting SIGLEC15.

### SUMMARY

This disclosure relates to anti-SIGLEC15 antibodies, antigen-binding fragment thereof, and the uses thereof.

In one aspect, the disclosure is related to an antibody or antigen-binding fragment thereof that binds to SIGLEC15 (Sialic acid binding ig-like lectin 15) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 1, 2, 3, respectively, and the selected VL CDRs 1, 2, 3 amino acid sequences are set forth in SEQ ID NOs: 4, 5, 6, respectively;
(2) the selected VH CDRs 1, 2, 3 amino acid sequences are set forth in SEQ ID NOs: 7, 8, 9, respectively, and the selected VL CDRs 1, 2, 3 amino acid sequences are set forth in SEQ ID NOs: 10, 11, 12, respectively;
(3) the selected VH CDRs 1, 2, 3 amino acid sequences are set forth in SEQ ID NOs: 13, 14, 15, respectively, and the selected VL CDRs 1, 2, 3 amino acid sequences are set forth in SEQ ID NOs: 16, 17, 18, respectively;
(4) the selected VH CDRs 1, 2, 3 amino acid sequences are set forth in SEQ ID NOs: 19, 20, 21, respectively, and the selected VL CDRs 1, 2, 3 amino acid sequences are set forth in SEQ ID NOs: 22, 23, 24, respectively;
(5) the selected VH CDRs 1, 2, 3 amino acid sequences are set forth in SEQ ID NOs: 25, 26, 27, respectively, and the selected VL CDRs 1, 2, 3 amino acid sequences are set forth in SEQ ID NOs: 28, 29, 30, respectively; and
(6) the selected VH CDRs 1, 2, 3 amino acid sequences are set forth in SEQ ID NOs: 31, 32, 33, respectively, and the selected VL CDRs 1, 2, 3 amino acid sequences are set forth in SEQ ID NOs: 34, 35, 36, respectively.

In some embodiments, the VH comprises CDRs 1, 2, 3 with the amino acid sequences set forth in SEQ ID NOs: 1, 2, and 3 respectively, and the VL comprises CDRs 1, 2, 3 with the amino acid sequences set forth in SEQ ID NOs: 4, 5, and 6, respectively, according to Kabat definition.

In some embodiments, the VH comprises CDRs 1, 2, 3 with the amino acid sequences set forth in SEQ ID NOs: 7, 8, and 9, respectively, and the VL comprises CDRs 1, 2, 3 with the amino acid sequences set forth in SEQ ID NOs: 10, 11, and 12, respectively, according to Kabat definition.

In some embodiments, the VH comprises CDRs 1, 2, 3 with the amino acid sequences set forth in SEQ ID NOs: 13, 14, and 15, respectively, and the VL comprises CDRs 1, 2, 3 with the amino acid sequences set forth in SEQ ID NOs: 16, 17, and 18, respectively, according to Kabat definition.

In some embodiments, the VH comprises CDRs 1, 2, 3 with the amino acid sequences set forth in SEQ ID NOs: 19, 20, and 21, respectively, and the VL comprises CDRs 1, 2, 3 with the amino acid sequences set forth in SEQ ID NOs: 22, 23, and 24, respectively, according to Chothia definition.

In some embodiments, the VH comprises CDRs 1, 2, 3 with the amino acid sequences set forth in SEQ ID NOs: 25, 26 and 27, respectively, and the VL comprises CDRs 1, 2, 3 with the amino acid sequences set forth in SEQ ID NOs: 28, 29, and 30, respectively, according to Chothia definition.

In some embodiments, the VH comprises CDRs 1, 2, 3 with the amino acid sequences set forth in SEQ ID NOs: 31, 32, and 33, respectively, and the VL comprises CDRs 1, 2, 3 with the amino acid sequences set forth in SEQ ID NOs: 34, 35, and 36, respectively, according to Chothia definition.

In some embodiments, the antibody or antigen-binding fragment thereof specifically binds to human, mouse, monkey, or dog SIGLEC15.

In some embodiments, the antibody or antigen-binding fragment thereof is a human antibody or antigen-binding fragment thereof, a single-chain variable fragment (scFv), a one-armed antibody, and/or a multi-specific antibody (e.g., a bispecific antibody).

In some embodiments, the antibody or antigen-binding fragment thereof is a human IgG1 antibody or antigen-binding fragment thereof or a human IgG4 antibody or antigen-binding fragment thereof.

In one aspect, the disclosure is related to a nucleic acid comprising a polynucleotide encoding a polypeptide comprising:
an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 1, 2, and 3, respectively, and wherein the VH, when paired with a light chain variable region (VL) comprising the amino acid sequence set forth in SEQ ID NO: 38 binds to SIGLEC15;
an immunoglobulin light chain or a fragment thereof comprising a VL comprising complementarity determining regions (CDRs) 1, 2, and 3 comprising the amino acid sequences set forth in SEQ ID NOs: 4, 5, and 6, respectively, and wherein the VL, when paired with a VH comprising the amino acid sequence set forth in SEQ ID NO: 37 binds to SIGLEC15;
an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 7, 8, and 9, respectively, and wherein the VH, when paired with a light chain variable region (VL) comprising the amino acid sequence set forth in SEQ ID NO: 40 binds to SIGLEC15;
an immunoglobulin light chain or a fragment thereof comprising a VL comprising complementarity determining regions (CDRs) 1, 2, and 3 comprising the amino acid sequences set forth in SEQ ID NOs: 10, 11, and 12, respectively, and wherein the VL, when paired with a VH comprising the amino acid sequence set forth in SEQ ID NO: 39 binds to SIGLEC15;
an immunoglobulin heavy chain or a fragment thereof comprising a VH comprising complementarity determining regions (CDRs) 1, 2, and 3 comprising the amino acid sequences set forth in SEQ ID NOs: 13, 14, and 15, respectively, and wherein the VH, when paired with a light chain variable region (VL) comprising the amino acid sequence set forth in SEQ ID NO: 42 binds to SIGLEC15; or
an immunoglobulin light chain or a fragment thereof comprising a VL comprising CDRs 1, 2, and 3 comprising the amino acid sequences set forth in SEQ ID NOs: 16, 17 and 18, respectively, and wherein the VL, when paired with a VH comprising the amino acid sequence set forth in SEQ ID NO: 41 binds to SIGLEC15.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1, 2, and 3, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 4, 5, and 6, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 7, 8, and 9, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 10, 11, and 12, respectively.

In some embodiments, the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 13, 14, and 15, respectively.

In some embodiments, the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 16, 17, and 18, respectively.

In some embodiments, the VH when paired with a VL specifically binds to human, mouse, monkey, or dog SIGLEC15, or the VL when paired with a VH specifically binds to human, mouse, monkey, or dog SIGLEC15.

In some embodiments, the immunoglobulin heavy chain or the fragment thereof is a human immunoglobulin heavy chain or a fragment thereof (e.g., a human IgG1 heavy chain or a fragment thereof; or a human IgG4 heavy chain or a fragment thereof), and the immunoglobulin light chain or the fragment thereof is a human immunoglobulin light chain or a fragment thereof.

In some embodiments, the nucleic acid encodes a single-chain variable fragment (scFv), a one-armed antibody, a multi-specific antibody (e.g., a bispecific antibody), or a chimeric antigen receptor (CAR). In some embodiments, the nucleic acid is cDNA.

In one aspect, the disclosure is related to a vector comprising one or more of the nucleic acids described herein.

In one aspect, the disclosure is related to a vector comprising two of the nucleic acids described herein, wherein the vector encodes the VL region and the VH region that together bind to SIGLEC15.

In one aspect, the disclosure is related to a pair of vectors, wherein each vector comprises one of the nucleic acids described herein, wherein together the pair of vectors encodes the VL region and the VH region that together bind to SIGLEC15.

In one aspect, the disclosure is related to a cell comprising the vector described herein, or the pair of vectors described herein.

In some embodiments, the cell is a CHO cell.

In one aspect, the disclosure is related to a cell comprising one or more of the nucleic acids described herein.

In one aspect, the disclosure is related to a cell comprising two of the nucleic acids described herein.

In some embodiments, the two nucleic acids together encode the VL region and the VH region that together bind to SIGLEC15.

In one aspect, the disclosure is related to a method of producing an antibody or an antigen-binding fragment thereof, the method comprising
(a) culturing the cell described herein under conditions sufficient for the cell to produce the antibody or the antigen-binding fragment; and
(b) collecting the antibody or the antigen-binding fragment produced by the cell.

In one aspect, the disclosure is related to an antibody or antigen-binding fragment thereof that binds to SIGLEC15 comprising a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are one of the following:
the selected VH sequence is SEQ ID NO: 37, and the selected VL sequence is SEQ ID NO: 38;
the selected VH sequence is SEQ ID NO: 39, and the selected VL sequence is SEQ ID NO: 40; and
the selected VH sequence is SEQ ID NO: 41, and the selected VL sequence is SEQ ID NO: 42.

In some embodiments, the VH comprises the sequence of SEQ ID NO: 37 and the VL comprises the sequence of SEQ ID NO: 38.

In some embodiments, the VH comprises the sequence of SEQ ID NO: 39 and the VL comprises the sequence of SEQ ID NO: 40.

In some embodiments, the VH comprises the sequence of SEQ ID NO: 41 and the VL comprises the sequence of SEQ ID NO: 42.

In some embodiments, the antibody or antigen-binding fragment thereof specifically binds to human, mouse, monkey, or dog SIGLEC15.

In some embodiments, the antibody or antigen-binding fragment thereof is a human antibody or antigen-binding fragment thereof, a single-chain variable fragment (scFv), a one-armed antibody, and/or a multi-specific antibody (e.g., a bispecific antibody).

In some embodiments, the antibody or antigen-binding fragment is a human IgG1 antibody or antigen-binding fragment thereof or a human IgG4 antibody or antigen-binding fragment thereof.

In one aspect, the disclosure is related to an antibody or antigen-binding fragment thereof comprising the VH CDRs 1, 2, 3, and the VL CDRs 1, 2, 3 of the antibody or antigen-binding fragment thereof described herein.

In one aspect, the disclosure is related to an antibody or antigen-binding fragment thereof that cross-competes with the antibody or antigen-binding fragment thereof described herein.

In some embodiments, the antibody or antigen-binding fragment thereof comprises a fragment crystallizable region (Fc region).

In some embodiments, the Fc region has reduced or no complement-dependent cytotoxicity (CDC) or antibody-dependent cellular cytotoxicity (ADCC).

In one aspect, the disclosure is related to a chimeric antigen receptor (CAR) comprising the antibody or antigen-binding fragment thereof described herein.

In one aspect, the disclosure is related to an antibody-drug conjugate comprising the antibody or antigen-binding fragment thereof described herein covalently bound to a therapeutic agent.

In some embodiments, the therapeutic agent is a cytotoxic or cytostatic agent.

In one aspect, the disclosure is related to a method of treating a subject having cancer, the method comprising administering a therapeutically effective amount of a composition comprising the antibody or antigen-binding fragment thereof described herein, or the antibody-drug conjugate described herein, to the subject.

In some embodiments, the subject has a solid tumor.

In some embodiments, the cancer is non-small-cell lung carcinoma (NSCLC), ovarian cancer, melanoma, colorectal cancer, breast cancer, colon adenocarcinoma, a hematological malignancy, head and neck cancer, gastrointestinal cancer, bladder cancer, or bone cancer.

In some embodiments, the cancer is Non-Hodgkin's lymphoma, lymphoma, leukemia, acute myeloid leukemia, or chronic lymphocytic leukemia.

In some embodiments, the subject is further treated with an effective amount of an anti-4-1BB antibody, an anti-OX40 antibody, anti-PD-1, anti-CTLA4, or an anti-PD-L1 antibody.

In one aspect, the disclosure is related to a method of decreasing the rate of tumor growth, the method comprising contacting a tumor cell with an effective amount of a composition comprising an antibody or antigen-binding fragment thereof described herein, or the antibody-drug conjugate described herein.

In one aspect, the disclosure is related to a method of killing a tumor cell, the method comprising contacting a tumor cell with an effective amount of a composition comprising the antibody or antigen-binding fragment thereof described herein, or the antibody-drug conjugate described herein.

In one aspect, the disclosure is related to a method of increasing immune response in a subject, the method comprising administering to the subject an effective amount of a composition comprising the antibody or antigen-binding fragment thereof described herein.

In one aspect, the disclosure is related to a method of treating a subject having a bone disease, the method comprising administering a therapeutically effective amount of a composition comprising the antibody or antigen-binding fragment thereof described herein to the subject.

In some embodiments, the bone disease is osteoporosis.

In one aspect, the disclosure is related to a pharmaceutical composition comprising the antibody or antigen-binding fragment thereof described herein, and a pharmaceutically acceptable carrier.

In one aspect, the disclosure is related to a pharmaceutical composition comprising the antibody drug conjugate described herein, and a pharmaceutically acceptable carrier.

As used herein, the term “cancer” refers to cells having the capacity for autonomous growth. Examples of such cells include cells having an abnormal state or condition characterized by rapidly proliferating cell growth. The term is meant to include cancerous growths, e.g., tumors; oncogenic processes, metastatic tissues, and malignantly transformed cells, tissues, or organs, irrespective of histopathologic type or stage of invasiveness. Also included are malignancies of the various organ systems, such as respiratory, cardiovascular, renal, reproductive, hematological, neurological, hepatic, gastrointestinal, and endocrine systems; as well as adenocarcinomas which include malignancies such as most colon cancers, renal-cell carcinoma, prostate cancer and/or testicular tumors, non-small cell carcinoma of the lung, and cancer of the small intestine. Cancer that is “naturally arising” includes any cancer that is not experimentally induced by implantation of cancer cells into a subject, and includes, for example, spontaneously arising cancer, cancer caused by exposure of a patient to a carcinogen(s), cancer resulting from insertion of a transgenic oncogene or knockout of a tumor suppressor gene, and cancer caused by infections, e.g., viral infections. The term “carcinoma” is art recognized and refers to malignancies of epithelial or endocrine tissues. The term also includes carcinosarcomas, which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation. The term “hematopoietic neoplastic disorders” includes diseases involving hyperplastic/neoplastic cells of hematopoietic origin. A hematopoietic neoplastic disorder can arise from myeloid, lymphoid or erythroid lineages, or precursor cells thereof.

As used herein, the term “antibody” refers to any antigen-binding molecule that contains at least one (e.g., one, two, three, four, five, or six) complementary determining region (CDR) (e.g., any of the three CDRs from an immunoglobulin light chain or any of the three CDRs from an immunoglobulin heavy chain) and is capable of specifically binding to an epitope. Non-limiting examples of antibodies include: monoclonal antibodies, polyclonal antibodies, multi-specific antibodies (e.g., bi-specific antibodies), single-chain antibodies, chimeric antibodies, human antibodies, and humanized antibodies. In some embodiments, an antibody can contain an Fc region of a human antibody. The term antibody also includes derivatives, e.g., bi-specific antibodies, single-chain antibodies, diabodies, linear antibodies, and multi-specific antibodies formed from antibody fragments.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain). Non-limiting examples of antibody fragments include, e.g., Fab, Fab′, F(ab′)₂, and Fv fragments.

As used herein, the term “human antibody” refers to an antibody that is encoded by an endogenous nucleic acid (e.g., rearranged human immunoglobulin heavy or light chain locus) present in a human. In some embodiments, a human antibody is collected from a human or produced in a human cell culture (e.g., human hybridoma cells). In some embodiments, a human antibody is produced in a non-human cell (e.g., a mouse or hamster cell line). In some embodiments, a human antibody is produced in a bacterial or yeast cell. In some embodiments, a human antibody is produced in a transgenic non-human animal (e.g., a bovine) containing an unrearranged or rearranged human immunoglobulin locus (e.g., heavy or light chain human immunoglobulin locus).

As used herein, the term “chimeric antibody” refers to an antibody that contains a sequence present in at least two different antibodies (e.g., antibodies from two different mammalian species such as a human and a mouse antibody). A non-limiting example of a chimeric antibody is an antibody containing the variable domain sequences (e.g., all or part of a light chain and/or heavy chain variable domain sequence) of a non-human (e.g., mouse) antibody and the constant domains of a human antibody. Additional examples of chimeric antibodies are described herein and are known in the art.

As used herein, the term “humanized antibody” refers to a non-human antibody which contains minimal sequence derived from a non-human (e.g., mouse) immunoglobulin and contains sequences derived from a human immunoglobulin. In non-limiting examples, humanized antibodies are human antibodies (recipient antibody) in which hypervariable (e.g., CDR) region residues of the recipient antibody are replaced by hypervariable (e.g., CDR) region residues from a non-human antibody (e.g., a donor antibody), e.g., a mouse, rat, or rabbit antibody, having the desired specificity, affinity, and capacity. In some embodiments, the Fv framework residues of the human immunoglobulin are replaced by corresponding non-human (e.g., mouse) immunoglobulin residues. In some embodiments, humanized antibodies may contain residues which are not found in the recipient antibody or in the donor antibody. These modifications can be made to further refine antibody performance. In some embodiments, the humanized antibody contains substantially all of at least one, and typically two, variable domains, in which all or substantially all of the hypervariable loops (CDRs) correspond to those of a non-human (e.g., mouse) immunoglobulin and all or substantially all of the framework regions are those of a human immunoglobulin. The humanized antibody can also contain at least a portion of an immunoglobulin constant region (Fc), typically, that of a human immunoglobulin. Humanized antibodies can be produced using molecular biology methods known in the art. Non-limiting examples of methods for generating humanized antibodies are described herein.

As used herein, the term “single-chain antibody” refers to a single polypeptide that contains at least two immunoglobulin variable domains (e.g., a variable domain of a mammalian immunoglobulin heavy chain or light chain) that is capable of specifically binding to an antigen. Non-limiting examples of single-chain antibodies are described herein.

As used herein, the term “multimeric antibody” refers to an antibody that contains four or more (e.g., six, eight, or ten) immunoglobulin variable domains. In some embodiments, the multimeric antibody is able to crosslink one target molecule (e.g., SIGLEC15) to at least one second target molecule on the surface of a mammalian cell (e.g., a human T-cell).

As used herein, the terms “subject” and “patient” are used interchangeably throughout the specification and describe an animal, human or non-human, to whom treatment according to the methods of the present invention is provided. Veterinary and non-veterinary applications are contemplated by the present invention. Human patients can be adult humans or juvenile humans (e.g., humans below the age of 18 years old). In addition to humans, patients include but are not limited to mice, rats, hamsters, guinea-pigs, rabbits, ferrets, cats, dogs, and primates. Included are, for example, non-human primates (e.g., monkey, chimpanzee, gorilla, and the like), rodents (e.g., rats, mice, gerbils, hamsters, ferrets, rabbits), lagomorphs, swine (e.g., pig, miniature pig), equine, canine, feline, bovine, and other domestic, farm, and zoo animals.

As used herein, when referring to an antibody, the phrases “specifically binding” and “specifically binds” mean that the antibody interacts with its target molecule (e.g., SIGLEC15) preferably to other molecules, because the interaction is dependent upon the presence of a particular structure (i.e., the antigenic determinant or epitope) on the target molecule; in other words, the reagent is recognizing and binding to molecules that include a specific structure rather than to all molecules in general. An antibody that specifically binds to the target molecule may be referred to as a target-specific antibody. For example, an antibody that specifically binds to a SIGLEC15 molecule may be referred to as a SIGLEC15-specific antibody or an anti-SIGLEC15 antibody.

As used herein, the terms “polypeptide,” “peptide,” and “protein” are used interchangeably to refer to polymers of amino acids of any length of at least two amino acids.

As used herein, the terms “polynucleotide,” “nucleic acid molecule,” and “nucleic acid sequence” are used interchangeably herein to refer to polymers of nucleotides of any length of at least two nucleotides, and include, without limitation, DNA, RNA, DNA/RNA hybrids, and modifications thereof.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1B show the exemplary structures of the antibodies described herein.

FIGS. 2A-2C show the serum levels of human antibodies over time as determined by sandwich ELISA.

FIG. 3 shows the tumor size data in groups treated with PBS, Atezolizumab analog at 3 mg/kg, 23F4 at 1 mg/kg, 3 mg/kg and 10 mg/kg, or a combination of Atezolizumab analog at 3 mg/kg and 23F4 at 10 mg/kg.

FIG. 4 shows the tumor size data in groups treated with PBS, 23F4 at 3 mg/kg or 5G12 at 10 mg/kg.

FIG. 5 shows the cell sorting strategy for TILs (tumor-infiltrating lymphocytes) analysis.

FIGS. 6A-6E show the results of TILs analysis in MC38 tumor model, as determined by flow cytometry.

FIG. 7 shows the tumor size data in groups treated with PBS, Atezolizumab analog at 1 mg/kg, 10A7 at 10 mg/kg, or a combination of Atezolizumab analog at 1 mg/kg and 10A7 at 10 mg/kg.

FIG. 8 shows the tumor size data in groups treated with PBS, YH004 at 0.3 mg/kg, 23F4 at 10 mg/kg, or a combination of YH004 at 0.3 mg/kg and 23F4 at 10 mg/kg.

FIG. 9A shows the average body weight of the mice in the control group and the treatment groups.

FIG. 9B shows the body weight change of the mice in the control group and the treatment groups.

FIGS. 10A-10M show the test results from the biochemical test (alanine transaminase (ALT) and aspartate (AST)) and hematology test (complete blood count, including white blood cell count (WBC), red blood cell count (RBC), hemoglobin (HGB), hematocrit or packed cell volume (HCT), mean corpuscle volume (MCV), mean corpuscular hemoglobin (MCH), mean corpuscular hemoglobin concentration (MCHC), platelet count (PLT), lymphocyte percentage (LYMPH %), monocyte percentage (MONO %), neutrophil percentage (NEUT %)).

FIG. 11 lists CDR sequences of anti-SIGLEC15 antibodies (10A7, 10C9, and 23F4) and CDR sequences of related anti-SIGLEC15 antibodies thereof as defined by Kabat numbering.

FIG. 12 lists CDR sequences of anti-SIGLEC15 antibodies (10A7, 10C9, and 23F4) and CDR sequences of related anti-SIGLEC15 antibodies thereof as defined by Chothia numbering.

FIG. 13 lists amino acid sequences of heavy chain variable regions and light chain variable regions of anti-SIGLEC15 antibodies (10A7, 10C9, and 23F4).

FIG. 14 lists certain amino acid sequences discussed in the disclosure.

### DETAILED DESCRIPTION

The present disclosure provides examples of antibodies, antigen-binding fragment thereof, that bind to SIGLEC15.

### SIGLEC15 and Immune System

SIGLEC15 (Sialic acid-binding immunoglobulin-like lectin 15) belongs to the SIGLEC family and is a type I transmembrane protein. It is rarely expressed in most normal human tissues and immune cell subsets, but has relatively high expression in macrophages.

SIGLEC constitute a family of cell surface proteins with an important role in the regulation of immune homeostasis. The dysregulation of these proteins has been associated with multiple diseases ranging from autoimmunity to infections and cancer. They are type I transmembrane proteins with one V-set immunoglobulin (Ig) domain containing the sialic acid-binding site and one or more C2-set Ig domains in their extracellular region. The majority of Siglecs, including CD22 (Siglec-2) and most CD33 (Siglec-3)-related Siglecs, have immunoreceptor tyrosine-based inhibitory motifs (ITIM) and/or ITIM-like motifs in their cytoplasmic domain and mediate inhibitory receptor signaling. Each Siglec preferentially recognizes a different kind of sialic acids, a group of sugars that are expressed on all mammalian cells as a mechanism to discriminate between self and nonself. But some pathogens can utilize inhibitory Siglecs to dampen the immune response and benefit their survival. Although most Siglecs work as receptors, some Siglecs can serve as functional ligands, such as Siglec-1. Siglec expression has been found mainly on hematopoietic cells (mostly on myeloid cell and B cells) or nonhematopoietic cells such as neurons. Among the Siglec family, Siglec-15 has been identified as a very unique member, selectively expressed on myeloid cells and osteoclasts (a bone-specific myeloid lineage) and generally absent in other immune cells and tissues.

The amino acid sequence alignment between human and mouse Siglec-15 shows 83% identity, and unlike other Siglecs that reside within Chromosome 19 or 1, Siglec-15 gene resides within Chromosome 18. Early studies suggested that Siglec-15 contains a conserved arginine (R143) motif in the membrane distal IgV domain, which is critical for sialic acid binding. Siglec-15 preferentially binds to Sialyl-Tn (Neu5Ac alpha 2-6GalNAc), a short O-glycan with a sialic acid residue whose neo- or over-expression is associated with various types of epithelial cancers. Importantly, as opposed to the majority of the Siglecs that contain one IgV domain and multiple “tandem repeats” of IgC2 domains in the extracellular region, Siglec-15 displays only one IgV and one IgC2 domain, which is commonly seen in B7 family members. There is high structural homology between Siglec-15 and PD-L1, and the protein sequence of Siglec-15's extracellular domain exhibits 20%-30% identity to B7 family, similar to the identity among B7 family members. These distinctive molecular features highlight the unique nature of Siglec-15 and suggest a possible link with B7 immune modulatory molecules.

Unlike the majority of Siglec members, Siglec-15 does not have typical ITIMs or ITIM-like motifs in its intracellular domain that mediate inhibitory signaling. Instead, it was reported to be associated with signaling adaptor DNAX-activating protein of 12 kDa (DAP12) and DAP10 that contain an immunoreceptor tyrosine-based activation motif (ITAM), through a positively charged lysine residue (K273 in mouse Siglec-15: K274 in human Siglec-15) in its transmembrane domain. The association with DAP12 and/or DAP10 is a typical feature of some Siglecs with activating signaling, such as Siglec-14 and Siglec-16, which is possibly achieved through the recruitment of spleen tyrosine kinase (SYK) and ZAP70 among others.

Siglec-15 has been identified as an important regulator in osteoclast differentiation and function. Yoshiharu and colleagues, in an attempt to identify regulators for osteoclast-like giant cell tumors, discovered that Siglec-15 was upregulated on osteoclasts upon stimulation by receptor activator of nuclear factor-KB ligand. Knockdown of Siglec-15 by shRNA or treatment with polyclonal antibodies against Siglec-15 inhibits osteoclast differentiation and bone resorption. Sialyl-acid/Siglec-15 axis may constitute a functional loop for osteoclast differentiation—removal of sialyl acids by sialidase or disruption of sialylated glycan binding by R143 mutation impaired osteoclast development. DAP12 may be needed for Siglec-15 function in osteoclasts, as the K273 mutation that disrupted the DAP12 association with Siglec-15 led to the function loss of Siglec-15 in osteoclasts. However, it is still unclear whether Siglec-15 mainly serves as a receptor or ligand, and how important DAP-12 and DAP-10 association is for Siglec-15's osteoclast function.

Given its structural similarities with B7 family and dominant expression pattern on myeloid cells, it was hypothesized that Siglec-15 might be involved in the regulation of immunity. In early 2010, using the TCAA screening platform, one group observed inhibition of NFκB reporter activity in Jurkat T cells by HEK-293T cells expressing Siglec-15. In addition, Siglec-15 ectodomain fusion protein either coated on plates or supplied in soluble form, robustly inhibited anti-CD3 (OKT3) induced human T-cell proliferation. In line with this, Siglec-15 expression on artificial APCs suppressed mouse T-cell proliferation, cytokine secretion, and killing capacity. These in vitro data suggested a ligand-like function of Siglec-15 that suppresses human or mouse T cells through unknown receptor(s) signaling. In vivo function of Siglec-15 on T cells was subsequently validated using an experimental autoimmune encephalomyelitis (EAE) mouse model. It was found that EAE was significantly aggravated in Siglec-15 deficient mice or by injecting Siglec-15 ectodomain fusion protein, and T-cell response was remarkably amplified in comparison with control groups. In addition, it was observed that Siglec-15 affects antigen-specific T-cell responses-Siglec-15-deficient mice showed a much higher OT-I T-cell expansion in the blood and spleen compared with WT mice upon OVA peptide stimulation, which resembles the phenotype in PD-L1 KO mice. In this process, IL10 might be an important factor since Siglec15-deficient mice showed decreased IL10 levels in serum compared with WT, and anti-IL 10 mAbs abrogated the differences in OT-I T-cells expansion between WT and Siglec-15-deficient mice.

A detailed review of SIGLEC15 and its functions can be found in Sun, Jingwei, et al. “Siglec-15 as an emerging target for next-generation cancer immunotherapy.” Clinical Cancer Research 27.3 (2021): 680-688, which is incorporated by reference in its entirety.

The present disclosure provides several anti-SIGLEC15 antibodies, antigen-binding fragments thereof, and methods of using these anti-SIGLEC15 antibodies and antigen-binding fragments to inhibit tumor growth, treat cancers, and to treat autoimmune diseases.

### Antibodies and Antigen Binding Fragments

The present disclosure provides anti-SIGLEC15 antibodies and antigen-binding fragments thereof. In general, antibodies (also called immunoglobulins) are made up of two classes of polypeptide chains, light chains and heavy chains. A non-limiting antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the principle antigen binding surface of the antibody. The four framework regions largely adopt a beta-sheet conformation and the CDRs form loops connecting, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132:211-250; Martin et al., Methods Enzymol. 203:121-53 (1991); Morea et al., Biophys Chem. 68 (1-3): 9-16 (October 1997); Morea et al., J Mol Biol. 275 (2): 269-94 (January 1998); Chothia et al., Nature 342 (6252): 877-83 (December 1989); Ponomarenko and Bourne, BMC Structural Biology 7:64 (2007); each of which is incorporated herein by reference in its entirety.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al. “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′)2, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the scFV has one heavy chain variable domain, and one light chain variable domain. In some embodiments, the scFV has two heavy chain variable domains, and two light chain variable domains.

In some embodiments, sequences (e.g., CDRs or VH/VL sequences) of the antibody or antigen-binding fragment thereof described herein can be used to generate a bispecific antibody targeting SIGLEC15 and an addition antigen (e.g., PD-1).

### Anti-SIGLEC15 Antibodies and Antigen-Binding Fragments

The disclosure provides antibodies and antigen-binding fragments thereof that specifically bind to SIGLEC15 (e.g., human SIGLEC15). The antibodies and antigen-binding fragments described herein are capable of binding to SIGLEC15. These antibodies can be agonists or antagonists. In some embodiments, these antibodies can increase immune response. In some embodiments, these antibodies can block SIGLEC15 pathway.

The disclosure provides e.g., anti-SIGLEC15 antibodies 10A7, 10C9, 23F4, the chimeric antibodies thereof, and the human or humanized antibodies thereof.

The CDR sequences for 10A7, and 10A7 derived antibodies (e.g., human or humanized antibodies) include CDRs of the heavy chain variable domain, SEQ ID NOs: 1-3, and CDRs of the light chain variable domain, SEQ ID NOs: 4-6 as defined by Kabat numbering. The CDRs can also be defined by Chothia system. Under the Chothia numbering, the CDR sequences of the heavy chain variable domain are set forth in SEQ ID NOs: 19-21 and CDR sequences of the light chain variable domain are set forth in SEQ ID NOs: 22-24.

Similarly, the CDR sequences for 10C9, and 10C9 derived antibodies include CDRs of the heavy chain variable domain, SEQ ID NOs: 7-9, and CDRs of the light chain variable domain, SEQ ID NOs: 10-12, as defined by Kabat numbering. Under Chothia numbering, the CDR sequences of the heavy chain variable domain are set forth in SEQ ID NOs: 25-27, and CDRs of the light chain variable domain are set forth in SEQ ID NOs: 28-30.

The CDR sequences for 23F4, and 23F4 derived antibodies include CDRs of the heavy chain variable domain, SEQ ID NOs: 13-15, and CDRs of the light chain variable domain, SEQ ID NOs: 16-18, as defined by Kabat numbering. Under Chothia numbering, the CDR sequences of the heavy chain variable domain are set forth in SEQ ID NOs: 31-33, and CDRs of the light chain variable domain are set forth in SEQ ID NOs: 34-36.

The amino acid sequence for the heavy chain variable region of 10A7 antibody is set forth in SEQ ID NO: 37. The amino acid sequence for the light chain variable region of 10A7 antibody is set forth in SEQ ID NO: 38.

The amino acid sequence for the heavy chain variable region of 10C9 antibody is set forth in SEQ ID NO: 39. The amino acid sequence for the light chain variable region of 10C9 antibody is set forth in SEQ ID NO: 40.

The amino acid sequence for the heavy chain variable region of 23F4 antibody is set forth in SEQ ID NO: 41. The amino acid sequence for the light chain variable region of 23F4 antibody is set forth in SEQ ID NO: 42.

The amino acid sequences for heavy chain variable regions and light variable regions of the modified antibodies are also provided. In some embodiments, the heavy chain variable region is at least 80%, 85%, 90%, 91%, 92%, 93%, 94%, 95%, 96%, 97%, 98%, or 99% identical to SEQ ID NO: 37, 39, or 41. In some embodiments, the light chain variable region is at least 80%, 85%, 90%, 91%, 92%, 93%, 94%, 95%, 96%, 97%, 98%, or 99% identical to SEQ ID NO: 38, 40, or 42. The heavy chain variable region sequence can be paired with the corresponding light chain variable region sequence, and together they bind to SIGLEC15.

Humanization percentage means the percentage identity of the heavy chain or light chain variable region sequence as compared to human antibody sequences in International Immunogenetics Information System (IMGT) database. The top hit means that the heavy chain or light chain variable region sequence is closer to a particular species than to other species. For example, top hit to human means that the sequence is closer to human than to other species. Top hit to human and Macaca fascicularis means that the sequence has the same percentage identity to the human sequence and the Macaca fascicularis sequence, and these percentages identities are highest as compared to the sequences of other species. In some embodiments, humanization percentage is greater than 80%, 81%, 82%, 83%, 84%, 85%, 86%, 87%, 88%, 89%, 90%, 91%, 92%, 93%, 94%, or 95%. A detailed description regarding how to determine humanization percentage and how to determine top hits is known in the art, and is described, e.g., in Jones, et al. “The INNs and outs of antibody nonproprietary names.” MAbs. Vol. 8. No. 1. Taylor & Francis, 2016, which is incorporated herein by reference in its entirety. A high humanization percentage often has various advantages, e.g., more safe and more effective in humans, more likely to be tolerated by a human subject, and/or less likely to have side effects. In some embodiments, the variable regions are fully human, e.g., derived from human heavy chain immunoglobulin locus sequences (e.g., recombination of human IGHV, human IGHD, and human IGHJ genes), and/or human kappa chain immunoglobulin locus sequences (e.g., recombination of human IGKV and human IGKJ genes).

Furthermore, in some embodiments, the antibodies or antigen-binding fragments thereof described herein can also contain one, two, or three heavy chain variable region CDRs selected from the group of SEQ ID NOs: 1-3, SEQ ID NOs: 7-9, SEQ ID NOs: 13-15, SEQ ID NOs: 19-21, SEQ ID NOs: 25-27, and SEQ ID NOs: 31-33; and/or one, two, or three light chain variable region CDRs selected from the group of SEQ ID NOs: 4-6, SEQ ID NOs: 10-12, SEQ ID NOs: 16-18, SEQ ID NOs: 22-24, SEQ ID NOs: 28-30, and SEQ ID NOs: 34-36.

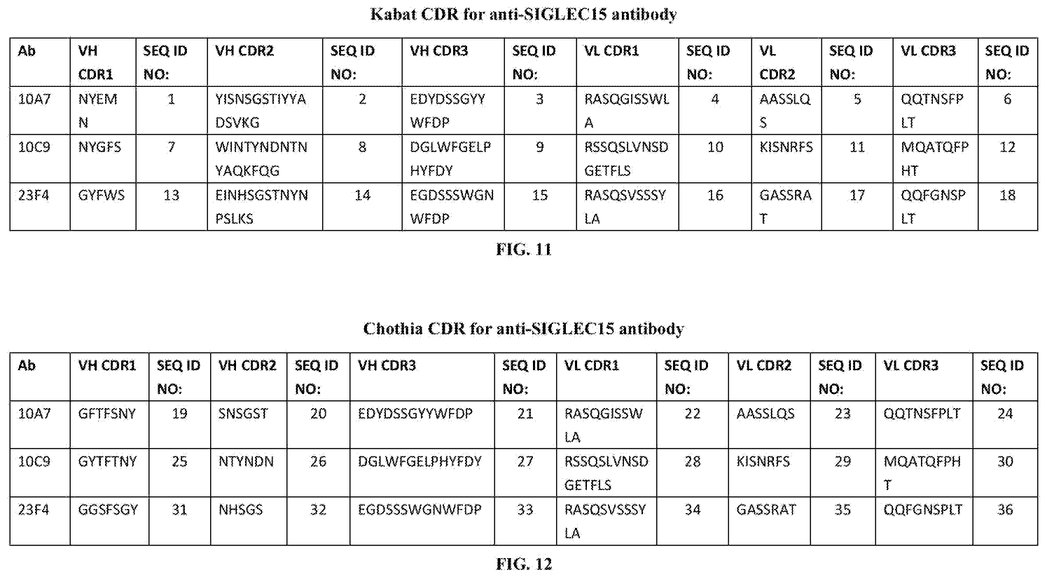

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence. In some embodiments, the antibody can have a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIG. 11 (Kabat CDR) and FIG. 12 (Chothia CDR).

In some embodiments, the antibody or an antigen-binding fragment described herein can contain a heavy chain variable domain containing one, two, or three of the CDRs of SEQ ID NO: 1 with zero, one or two amino acid insertions, deletions, or substitutions; SEQ ID NO: 2 with zero, one or two amino acid insertions, deletions, or substitutions; SEQ ID NO: 3 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 7 with zero, one or two amino acid insertions, deletions, or substitutions; SEQ ID NO: 8 with zero, one or two amino acid insertions, deletions, or substitutions; SEQ ID NO: 9 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 13 with zero, one or two amino acid insertions, deletions, or substitutions; SEQ ID NO: 14 with zero, one or two amino acid insertions, deletions, or substitutions; SEQ ID NO: 15 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 19 with zero, one or two amino acid insertions, deletions, or substitutions; SEQ ID NO: 20 with zero, one or two amino acid insertions, deletions, or substitutions; SEQ ID NO: 21 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 25 with zero, one or two amino acid insertions, deletions, or substitutions; SEQ ID NO: 26 with zero, one or two amino acid insertions, deletions, or substitutions; SEQ ID NO: 27 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 31 with zero, one or two amino acid insertions, deletions, or substitutions; SEQ ID NO: 32 with zero, one or two amino acid insertions, deletions, or substitutions; SEQ ID NO: 33 with zero, one or two amino acid insertions, deletions, or substitutions.

In some embodiments, the antibody or an antigen-binding fragment described herein can contain a light chain variable domain containing one, two, or three of the CDRs of SEQ ID NO: 4 with zero, one or two amino acid insertions, deletions, or substitutions; SEQ ID NO: 5 with zero, one or two amino acid insertions, deletions, or substitutions; SEQ ID NO: 6 with zero, one or two amino acid insertions, deletions, or substitutions.

In some embodiments, the antibody or an antigen-binding fragment described herein can contain a light chain variable domain containing one, two, or three of the CDRs of SEQ ID NO: 10 with zero, one or two amino acid insertions, deletions, or substitutions; SEQ ID NO: 11 with zero, one or two amino acid insertions, deletions, or substitutions; SEQ ID NO: 12 with zero, one or two amino acid insertions, deletions, or substitutions.

In some embodiments, the antibody or an antigen-binding fragment described herein can contain a light chain variable domain containing one, two, or three of the CDRs of SEQ ID NO: 16 with zero, one or two amino acid insertions, deletions, or substitutions; SEQ ID NO: 17 with zero, one or two amino acid insertions, deletions, or substitutions; SEQ ID NO: 18 with zero, one or two amino acid insertions, deletions, or substitutions.

In some embodiments, the antibody or an antigen-binding fragment described herein can contain a light chain variable domain containing one, two, or three of the CDRs of SEQ ID NO: 22 with zero, one or two amino acid insertions, deletions, or substitutions; SEQ ID NO: 23 with zero, one or two amino acid insertions, deletions, or substitutions; SEQ ID NO: 24 with zero, one or two amino acid insertions, deletions, or substitutions.

In some embodiments, the antibody or an antigen-binding fragment described herein can contain a light chain variable domain containing one, two, or three of the CDRs of SEQ ID NO: 28 with zero, one or two amino acid insertions, deletions, or substitutions; SEQ ID NO: 29 with zero, one or two amino acid insertions, deletions, or substitutions; SEQ ID NO: 30 with zero, one or two amino acid insertions, deletions, or substitutions.

In some embodiments, the antibody or an antigen-binding fragment described herein can contain a light chain variable domain containing one, two, or three of the CDRs of SEQ ID NO: 34 with zero, one or two amino acid insertions, deletions, or substitutions; SEQ ID NO: 35 with zero, one or two amino acid insertions, deletions, or substitutions; SEQ ID NO: 36 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat numbering scheme. In some embodiments, the CDR is determined based on Chothia numbering scheme. In some embodiments, the CDR is determined based on a combination of Kabat and Chothia numbering scheme.

The disclosure also provides antibodies or antigen-binding fragments thereof that bind to SIGLEC15. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 37, and the selected VL sequence is SEQ ID NO: 38. In some embodiments, the selected VH sequence is SEQ ID NO: 39 and the selected VL sequence is SEQ ID NO: 40. In some embodiments, the selected VH sequence is SEQ ID NO: 41, and the selected VL sequence is SEQ ID NO: 42.

The disclosure also provides antibodies or antigen-binding fragments thereof that can compete with the antibodies described herein. In some aspects, the antibodies or antigen-binding fragments can bind to the same epitope as the antibodies described herein.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

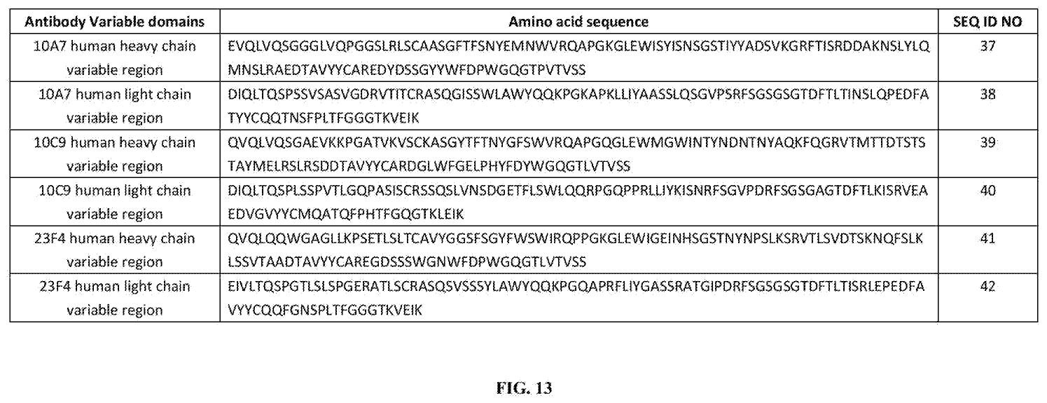

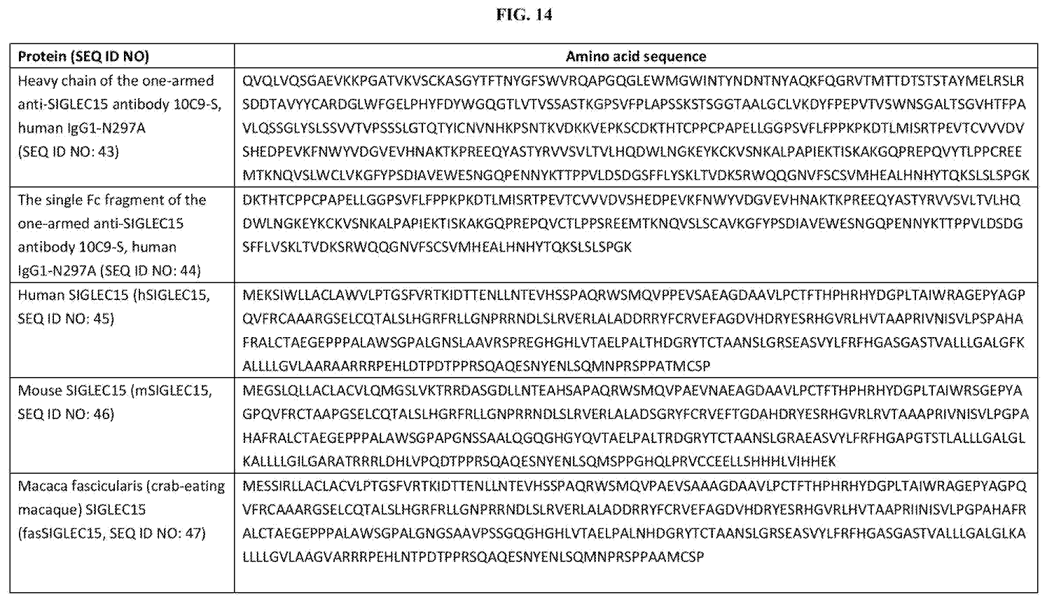

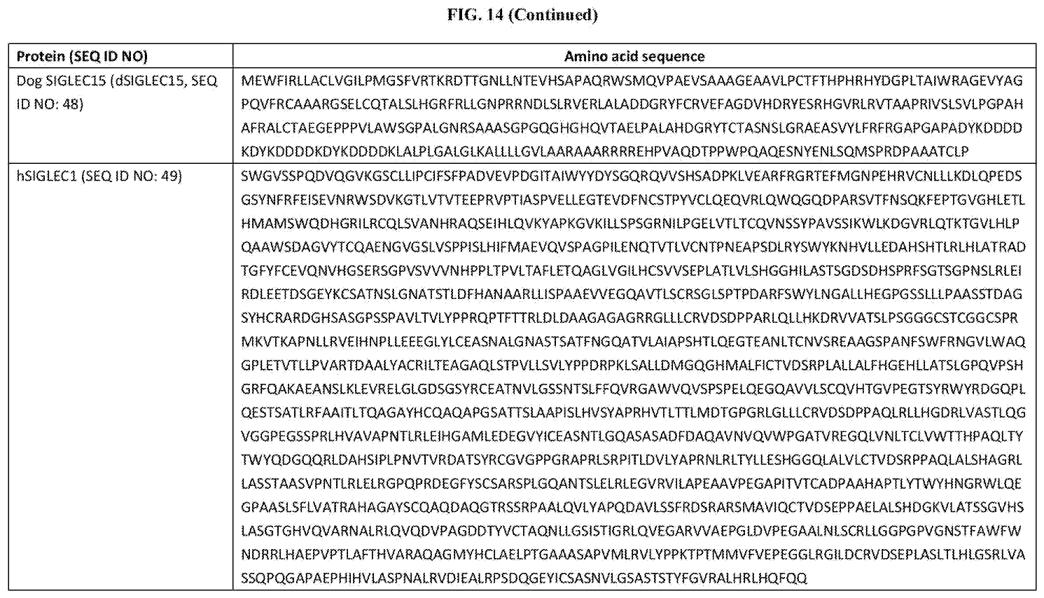

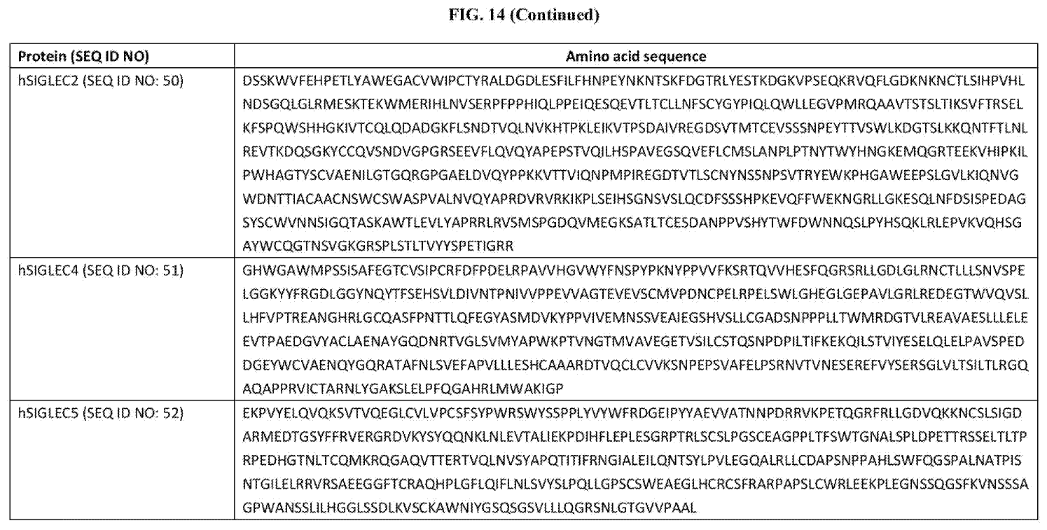

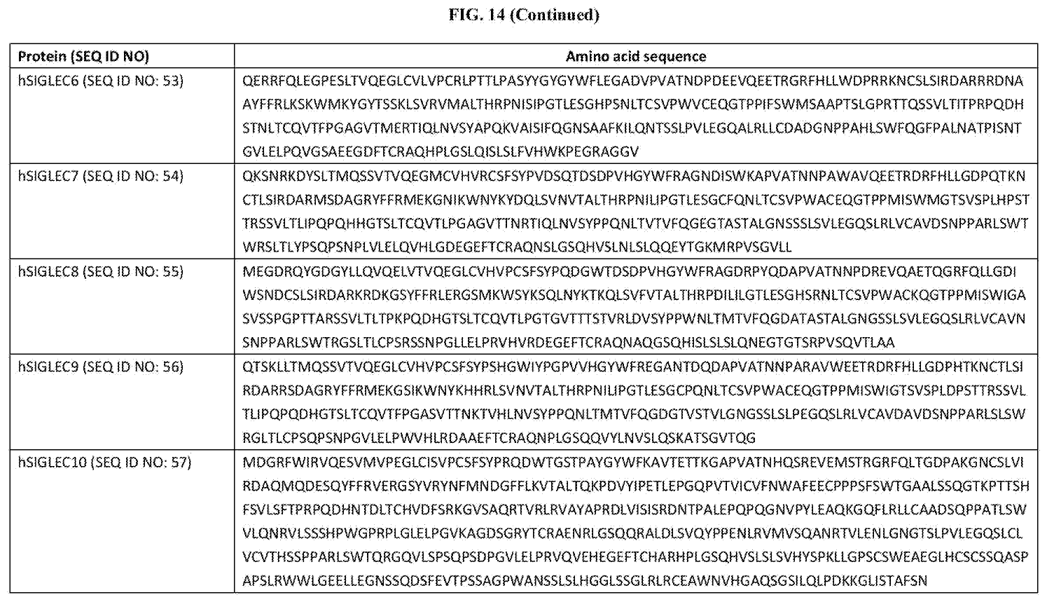

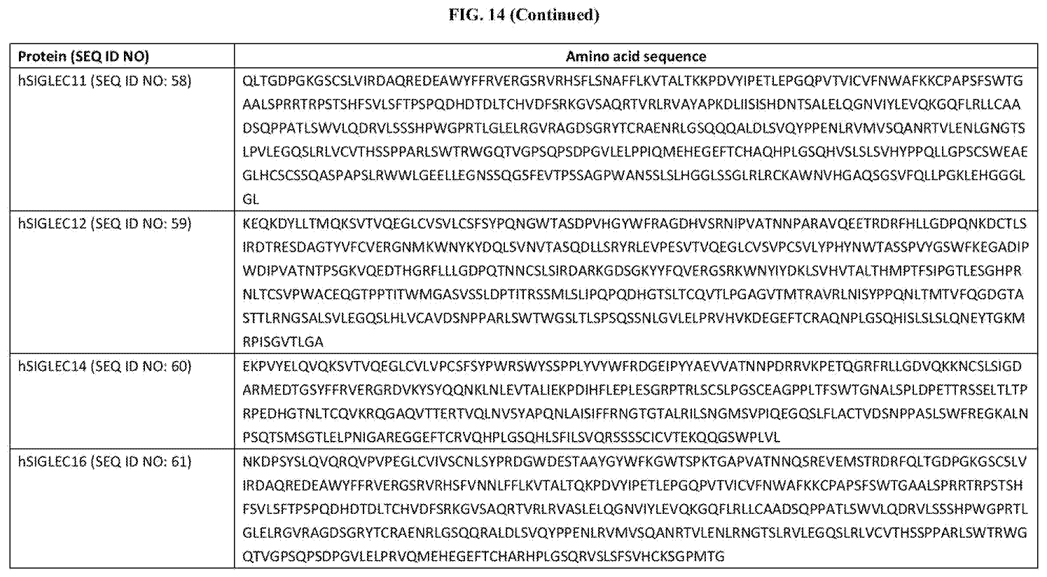

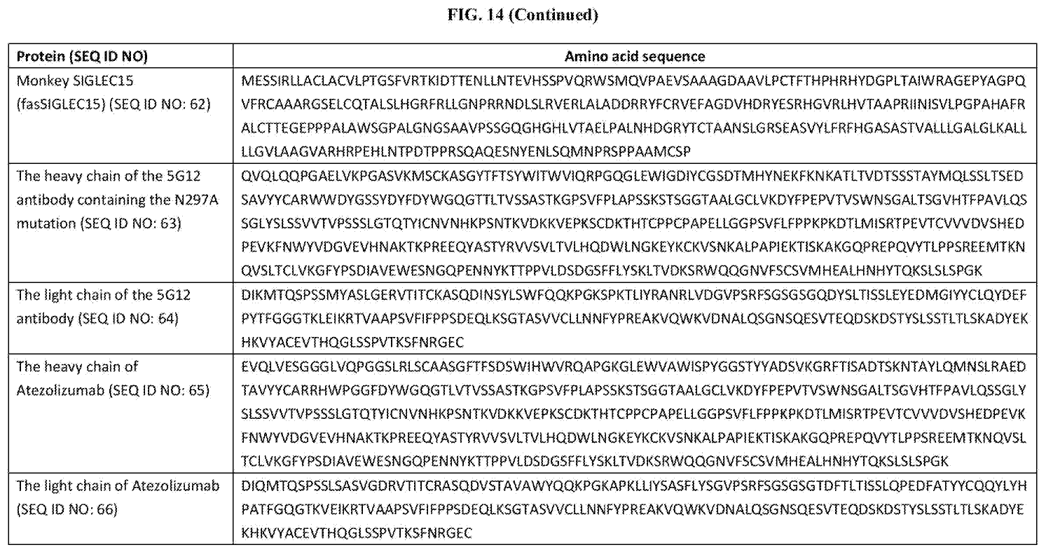

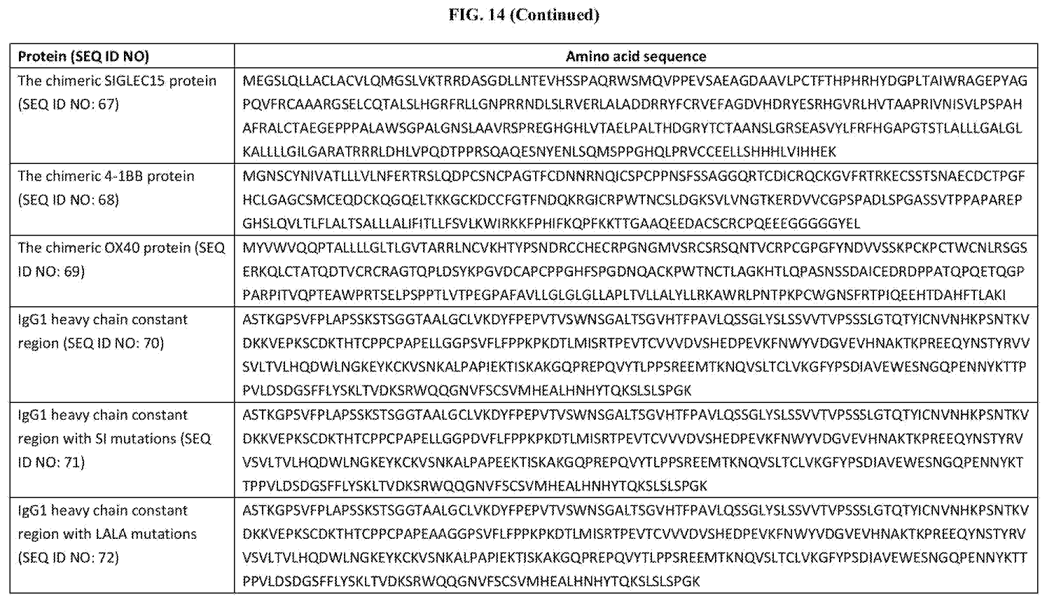

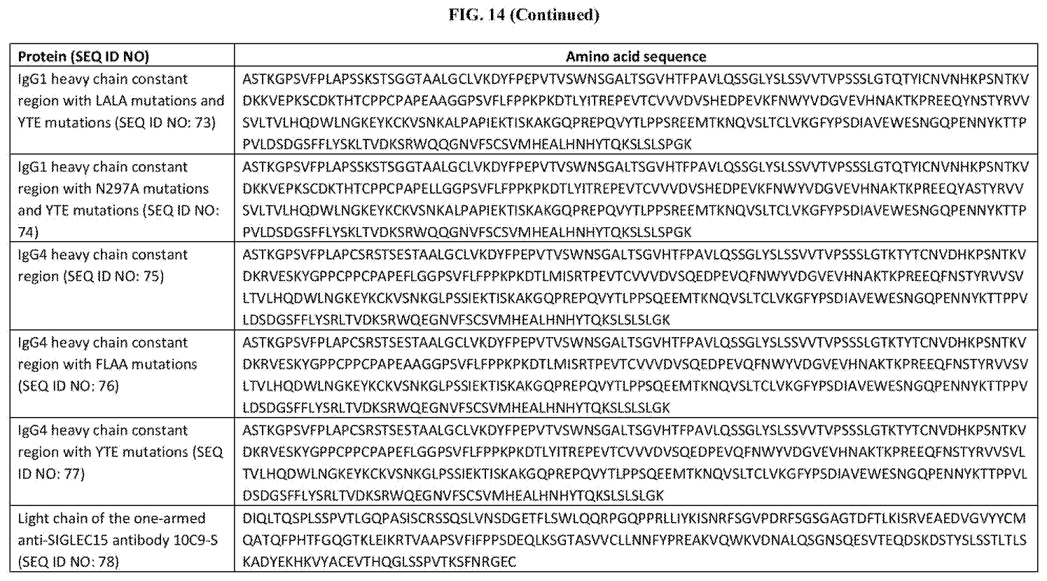

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs as shown in FIG. 11 or FIG. 12, or have sequences as shown in FIG. 13. When the polypeptides are paired with corresponding polypeptide (e.g., a corresponding heavy chain variable region or a corresponding light chain variable region), the paired polypeptides bind to SIGLEC15 (e.g., human SIGLEC15).

The anti-SIGLEC15 antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multi-specific (multimeric, e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to SIGLEC15 will retain an ability to bind to SIGLEC15. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site.

Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

The Fab fragment contains a variable and constant domain of the light chain and a variable domain and the first constant domain (CH1) of the heavy chain. F(ab′)2 antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Linear antibodies comprise a pair of tandem Fd segments (VH-CH1-VH-CH1) which, together with complementary light chain polypeptides, form a pair of antigen binding regions. Linear antibodies can be bispecific or monospecific.

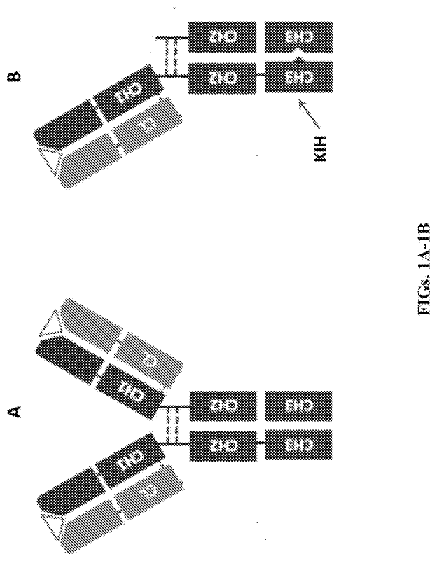

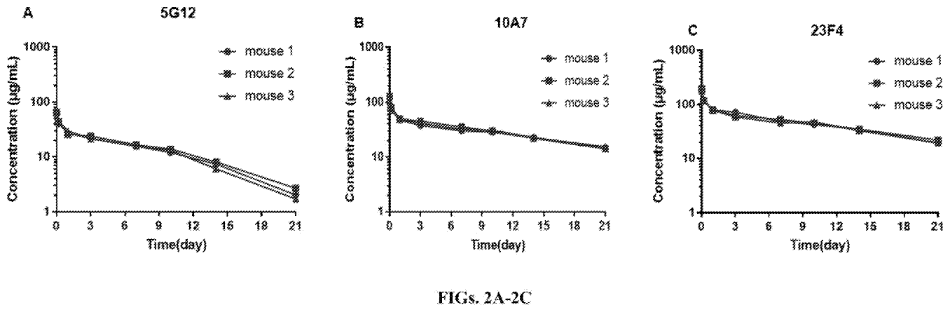

One-armed antibodies can have a heavy chain and a light chain, and a heavy chain fragment comprising CH2 and CH3 domains of IgG (FIG. 1B). In some embodiments, a one-armed antibody is an antibody that only has one of the two antigen binding arms in a typical antibody. In some embodiments, a one-armed antibody comprises an antigen binding arm (e.g., VH+CH1 and VL+CL), and a Fc.

Antibodies and antibody fragments of the present disclosure can be modified in the Fc region to provide desired effector functions or serum half-life. In some embodiments, the Fc region can be modified to silence or decrease complement-dependent cytotoxicity (CDC) or antibody-dependent cellular cytotoxicity (ADCC).

Multimerization of antibodies may be accomplished through natural aggregation of antibodies or through chemical or recombinant linking techniques known in the art. For example, some percentage of purified antibody preparations (e.g., purified IgG1 molecules) spontaneously form protein aggregates containing antibody homodimers and other higher-order antibody multimers.

Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) can be used to form antibody multimers. An exemplary protocol for the formation of antibody homodimers is described in Ghetie et al. (Proc. Natl. Acad. Sci. U.S.A. 94:7509-7514, 1997). Antibody homodimers can be converted to Fab′₂ homodimers through digestion with pepsin. Another way to form antibody homodimers is through the use of the autophilic T15 peptide described in Zhao et al. (J. Immunol. 25:396-404, 2002).

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Bi-specific antibodies include cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

Methods for generating bi-specific antibodies from antibody fragments are also known in the art. For example, bi-specific antibodies can be prepared using chemical linkage. Brennan et al. (Science 229:81, 1985) describes a procedure where intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′ TNB derivatives is then reconverted to the Fab′ thiol by reduction with mercaptoethylamine, and is mixed with an equimolar amount of another Fab′ TNB derivative to form the bi-specific antibody.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, tenoposide, vincristine, vinblastine, colchicin, doxorubicin, daunorubicin, dihydroxy anthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

In some embodiments, the antibody or antigen-binding fragment thereof described herein recognizes an endogenous SIGLEC15 or a recombinant SIGLEC15. In some embodiments, the antibody or antigen-binding fragment thereof described herein recognizes human SIGLEC15.

In some embodiments, the half-life of the antibody or antigen-binding fragment thereof described herein in wild-type mice (e.g., C57BL/6 mice) is at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, or at least 18 days. In some embodiments, the half-life of the antibody or antigen-binding fragment thereof described herein in SIGLEC15 gene humanized mice (e.g., hSIGLEC15 mice) is at least 1 day, at least 2 days, at least 3 days, at least 4 days, or at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, or at least 18 days. In some embodiments, the max concentration (Cmax) of the antibody or antigen-binding fragment thereof described herein in SIGLEC15 gene humanized mice (e.g., hSIGLEC15 mice) is at least 50 μg/ml, at least 75 μg/ml, at least 100 μg/ml, at least 125 μg/ml, at least 150 μg/ml, at least 175 μg/ml, or at least 200 μg/ml. In some embodiments, the serum clearance rate (CL) of the antibody or antigen-binding fragment thereof described herein in SIGLEC15 gene humanized mice (e.g., hSIGLEC15 mice) is at least 7.5 ml/day/kg, at least 10 ml/day/kg, at least 12.5 ml/day/kg, at least 15 ml/day/kg, at least 17.5 ml/day/kg, at least 20 ml/day/kg, at least 25 ml/day/kg, at least 30 ml/day/kg, at least 35 ml/day/kg, or at least 40 ml/day/kg.

In some embodiments, the clearance rate (CL) of the antibody or antigen-binding fragment thereof described herein in wild-type mice (e.g., C57BL/6 mice) is less than 7 ml/day/kg, less than 6 ml/day/kg, less than 5 ml/day/kg, or less than 4 ml/day/kg. In some embodiments, the clearance rate (CL) of the antibody or antigen-binding fragment thereof described herein in SIGLEC15 gene humanized mice (e.g., hSIGLEC15 mice) is less than 15 ml/day/kg, less than 14 ml/day/kg, less than 13 ml/day/kg, or less than 12 ml/day/kg.

In some embodiments, the half-life of the antibody or antigen-binding fragment thereof described herein (e.g., in FcRn gene humanized mice) is at least 6 days, at least 7 days, at least 8 days, at least 9 days, at least 10 days, at least 11 days, at least 12 days, at least 13 days, or at least 14 days. In some embodiments, the clearance rate (CL) of the antibody or antigen-binding fragment thereof described herein (e.g., in FcRn gene humanized mice) is less than 16 ml/day/kg, less than 15 ml/day/kg, less than 14 ml/day/kg, less than 13 ml/day/kg, less than 12 ml/day/kg, less than 11 ml/day/kg, less than 10 ml/day/kg, less than 9 ml/day/kg, less than 8 ml/day/kg, or less than 7 ml/day/kg.

### Antibody Characteristics

The antibodies or antigen-binding fragments thereof described herein can block the binding between SIGLEC15 and SIGLEC15 ligands (e.g., sialic acid).

The antibodies or antigen-binding fragments thereof as described herein can be SIGLEC15 pathway agonist or antagonist. In some embodiments, by binding to SIGLEC15, the antibody can inhibit SIGLEC15 signaling pathway. In some embodiments, the antibody can upregulate immune response or downregulate immune response.

In some embodiments, the antibodies or antigen-binding fragments thereof as described herein can increase immune response, activity or number of immune cells (e.g., T cells, CD8+ T cells, CD4+ T cells, macrophages, antigen presenting cells) by at least 10%, 20%, 30%, 40%, 50%, 60%, 70%, 80%, 90%, 100%, 2 folds, 3 folds, 5 folds, 10 folds, or 20 folds. In some embodiments, the antibodies or antigen-binding fragments thereof as described herein can decrease the activity or number of immune cells (e.g., T cells, CD8+ T cells, CD4+ T cells, macrophages, antigen presenting cells) by at least 10%, 20%, 30%, 40%, 50%, 60%, 70%, 80%, 90%, 100%, 2 folds, 3 folds, 5 folds, 10 folds, or 20 folds.

In some embodiments, the antibodies or antigen-binding fragments thereof as described herein can decrease the number of mCD45+ cells, e.g., by at least 10%, 20%, 30%, 40%, 50%, 60%, 70%, 80%, or 90%. In some embodiments, the antibodies or antigen-binding fragments thereof as described herein can decrease the amount of G-MDSC in mCD45+ cells, e.g., by at least 10%, 20%, 30%, 40%, 50%, 60%, 70%, 80%, or 90%. In some embodiments, the antibodies or antigen-binding fragments thereof as described herein can increase the amount of M-MDSC in mCD45+ cells, e.g., by at least 10%, 20%, 30%, 40%, 50%, 60%, 70%, 80%, 90%, 100%, 2 folds, 3 folds, 5 folds, 10 folds. In some embodiments, the antibodies or antigen-binding fragments thereof as described herein can increase the amount of CTL in mCD45+ cells, e.g., by at least 10%, 20%, 30%, 40%, 50%, 60%, 70%, 80%, 90%, 100%, 2 folds, 3 folds, 5 folds, 10 folds. In some embodiments, the antibodies or antigen-binding fragments thereof as described herein can increase the amount of Th cells in mCD45+ cells, e.g., by at least 10%, 20%, 30%, 40%, 50%, 60%, 70%, 80%, 90%, 100%, 2 folds, 3 folds, 5 folds, 10 folds.

In some implementations, the antibody (or antigen-binding fragments thereof) specifically binds to SIGLEC15 (e.g., human SIGLEC15, monkey SIGLEC15 (e.g., rhesus macaques, Macaca fascicularis), dog SIGLEC15, mouse SIGLEC15, and/or chimeric SIGLEC15) with a dissociation rate (koff) of less than 0.1 s⁻¹, less than 0.01 s⁻¹, less than 0.001 s 1, less than 0.0001 s⁻¹, less than 0.00001 s⁻¹, less than 0.000001 s⁻¹ or less than 0.0000001 s⁻¹. In some embodiments, the dissociation rate (koff) is greater than 0.01 s⁻¹, greater than 0.001 s⁻¹, greater than 0.0001 s⁻¹, greater than 0.00001 s⁻¹, greater than 0.000001 s⁻¹, greater than 0.0000001 s⁻¹ or greater than 0.00000001 s⁻¹.

In some embodiments, kinetic association rates (kon) is greater than 1×10²/Ms, greater than 1×10³/Ms, greater than 1×10⁴/Ms, greater than 1×10⁵/Ms, or greater than 1×10⁶/Ms. In some embodiments, kinetic association rates (kon) is less than 1×10⁵/Ms, less than 1×10⁶/Ms, or less than 1×10⁷/Ms.

Affinities can be deduced from the quotient of the kinetic rate constants (KD=koff/kon). In some embodiments, KD is less than 1×10⁻⁶ M, less than 1×10⁻⁷ M, less than 1×10⁻⁸ M, less than 1×10⁻⁹ M, less than 1×10⁻¹⁰ M, less than 1×10⁻¹¹ M, less than 1×10⁻¹² M, less than 1×10⁻¹³ M or less than 1×10⁻¹⁴ M. In some embodiments, the KD is less than 50 nM, 30 nM, 20 nM, 15 nM, 10 nM, 9 nM, 8 nM, 7 nM, 6 nM, 5 nM, 4 nM, 3 nM, 2 nM, or 1 nM. In some embodiments, KD is greater than 1×10⁻⁷ M, greater than 1×10⁻⁸ M, greater than 1×10⁻⁹ M, greater than 1×10⁻¹⁰ M, greater than 1×10⁻¹¹ M, greater than 1×10⁻¹² M, greater than 1×10⁻¹³ M, greater than 1×10⁻¹⁴ M.

General techniques for measuring the affinity of an antibody for an antigen include, e.g., ELISA, RIA, and surface plasmon resonance (SPR). In some embodiments, the antibody binds to human SIGLEC15 (SEQ ID NO: 45), mouse SIGLEC15 (SEQ ID NO: 46), monkey SIGLEC15 (SEQ ID NO: 47), dog SIGLEC15 (SEQ ID NO: 48) and/or chimeric SIGLEC15 (SEQ ID NO: 67). In some embodiments, the antibody does not bind to human SIGLEC15, mouse SIGLEC15, monkey SIGLEC15, dog SIGLEC15 and/or chimeric SIGLEC15.

In some embodiments, thermal stabilities are determined. The antibodies or antigen binding fragments as described herein can have a Tm greater than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C. In some embodiments, Tm is less than 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95° C.

In some embodiments, the antibodies or antigen-binding fragments thereof as described herein can bind to the same epitope of SIGLEC15. In some embodiments, the antibodies or antigen-binding fragments thereof as described herein can bind to different epitopes of SIGLEC15.

In some embodiments, the antibodies or antigen-binding fragments thereof as described herein have a purity of above 90%, above 91%, above 92%, above 93%, above 94%, above 95%, above 96%, above 97%, or above 98%, as determined by size exclusion chromatography (SEC). In some embodiments, the antibodies or antigen-binding fragments thereof as described herein have a hydrophobic interaction chromatography (HIC) retention time that is higher than 2 minutes, higher than 2.5 minutes, higher than 3 minutes, higher than 3.5 minutes, higher than 4 minutes, or higher than 4.5 minutes. In some embodiments, the antibodies or antigen-binding fragments thereof as described herein have a main peak that constitutes more than 50%, more than 55%, more than 60%, more than 65%, more than 70%, more than 75%, more than 80%, more than 85%, or more than 90%, as determined by capillary isoelectric focusing (cIEF). In some embodiments, the antibodies or antigen-binding fragments thereof as described herein have a isoelectric point (PI) that is higher than 7.5, higher than 7.75, higher than 8, higher than 8.25, higher than 8.5, or higher than 8.75, as determined by capillary isoelectric focusing (cIEF). In some embodiments, the antibodies or antigen-binding fragments thereof as described herein have a half maximal effective concentration (EC50) that is lower than 2.5, lower than 2, lower than 1.5, lower than 1, lower than 0.75, lower than 0.5, lower than 0.25, lower than 0.15 μg/mL.

In some embodiments, the antibodies or antigen-binding fragments thereof as described herein can release the inhibition of cell proliferation caused by hSIGLEC15. In some embodiments, the antibodies or antigen-binding fragments thereof as described herein can release the inhibition of hCD4+ T cell proliferation caused by hSIGLEC15 by more than 10%, more than 20%, more than 30%, more than 40%, more than 50%, more than 60%, or more than 70%. In some embodiments, the antibodies or antigen-binding fragments thereof as described herein can release the inhibition of hCD8+ T cell proliferation caused by hSIGLEC15 by more than 10%, more than 20%, more than 30%, more than 40%, more than 50%, more than 60%, or more than 70%.

In some embodiments, the antibody has a tumor growth inhibition percentage (TGI %) that is greater than 10%, 20%, 30%, 40%, 50%, 60%, 70%, 80%, 90%, 100%, 110%, 120%, 130%, 140%, 150%, 160%, 170%, 180%, 190%, or 200%. In some embodiments, the antibody has a tumor growth inhibition percentage that is less than 60%, 70%, 80%, 90%, 100%, 110%, 120%, 130%, 140%, 150%, 160%, 170%, 180%, 190%, or 200%. The TGI % can be determined, e.g., at 3, 4, 5, 6, 7, 8, 9, 10, 11, 12, 13, 14, 15, 16, 17, 18, 19, 20, 21, 22, 23, 24, 25, 26, 27, 28, 29, or 30 days after the treatment starts, or 1, 2, 3, 4, 5, 6, 7, 8, 9, 10, 11, or 12 months after the treatment starts. As used herein, the tumor growth inhibition percentage (TGI %) is calculated using the following formula:

$$TGI ( \% ) = [ 1 - ( Ti - T 0 ) / ( Vi - V 0 ) ] \times 100$$

Ti is the average tumor volume in the treatment group on day i. TO is the average tumor volume in the treatment group on day zero. Vi is the average tumor volume in the control group on day i. V0 is the average tumor volume in the control group on day zero.

In some embodiments, the antibodies or antigen-binding fragments thereof as described herein are SIGLEC15 antagonist. In some embodiments, the antibodies or antigen binding fragments decrease SIGLEC15 signal transduction in a target cell that expresses SIGLEC15.

In some embodiments, the antibodies or antigen binding fragments can enhance APC (e.g., DC cell) function, for example, inducing surface expression of costimulatory and MHC molecules, inducing production of proinflammatory cytokines, and/or enhancing T cell triggering function.

In some embodiments, the antibodies or antigen binding fragments can bind to tumor cells that express SIGLEC15. In some embodiments, the antibodies or antigen binding fragments can induce complement-dependent cytotoxicity (CDC) and/or antibody dependent cellular cytoxicity (ADCC), and kill the tumor cell.

In some embodiments, the antibodies or antigen binding fragments have a functional Fc region. In some embodiments, effector function of a functional Fc region is antibody-dependent cell-mediated cytotoxicity (ADCC). In some embodiments, effector function of a functional Fc region is phagocytosis. In some embodiments, effector function of a functional Fc region is ADCC and phagocytosis.

In some embodiments, the antibodies or antigen binding fragments can induce complement complement-dependent cytotoxicity (CDC).

In some embodiments, the Fc region is human IgG1, human IgG2, human IgG3, or human IgG4. In some embodiments, the antibody is a human IgG1 antibody, optionally with SI mutations, LALA mutations, N297A mutation, YTE mutations, and/or FLAA mutations. In some embodiments, the antibody is a human IgG4 antibody, optionally with SI mutations, LALA mutations, N297A mutation, YTE mutations, and/or FLAA mutations.

In some embodiments, the antibodies or antigen binding fragments do not have a functional Fc region. For example, the antibodies or antigen binding fragments are Fab, Fab′, F(ab′)₂, and Fv fragments. In some embodiments, the Fc region has LALA mutations (L234A and L235A mutations according to EU numbering), or LALA-PG mutations (L234A, L235A, P329G mutations according to EU numbering). In some embodiments, the Fc region has FLAA mutations (F234A and L235A according to EU numbering). In some embodiments, the Fc has SI mutations (S239D and I332E mutations according to EU numbering). In some embodiments, the Fc has N297A mutation according to EU numbering. In some embodiments, the Fc has YTE mutations (M252Y, S254T and T256E according to EU numbering).

In some embodiments, the antibodies or antigen binding fragments described herein include an amino acid sequence that is at least 80%, at least 85%, at least 90%, at least 95% or 100% identical to any one of SEQ ID NOs: 70-77.

### Methods of Making Anti-SIGLEC15 Antibodies

An isolated fragment of human SIGLEC15 can be used as an immunogen to generate antibodies using standard techniques for polyclonal and monoclonal antibody preparation. Polyclonal antibodies can be raised in animals by multiple injections (e.g., subcutaneous or intraperitoneal injections) of an antigenic peptide or protein. In some embodiments, the antigenic peptide or protein is injected with at least one adjuvant. In some embodiments, the antigenic peptide or protein can be conjugated to an agent that is immunogenic in the species to be immunized. Animals can be injected with the antigenic peptide or protein more than one time (e.g., twice, three times, or four times).

The full-length polypeptide or protein can be used or, alternatively, antigenic peptide fragments thereof can be used as immunogens. The antigenic peptide of a protein comprises at least 8 (e.g., at least 10, 15, 20, or 30) amino acid residues of the amino acid sequence of SIGLEC15 and encompasses an epitope of the protein such that an antibody raised against the peptide forms a specific immune complex with the protein. As described above, the full length sequence of human SIGLEC15 (SEQ ID NO: 45) is known in the art. In some embodiments, an Fc-tagged or His-tagged human SIGLEC15 protein is used as the immunogen.

An immunogen typically is used to prepare antibodies by immunizing a suitable subject (e.g., human or transgenic animal expressing at least one human immunoglobulin locus). An appropriate immunogenic preparation can contain, for example, a recombinantly-expressed or a chemically-synthesized polypeptide (e.g., a fragment of human SIGLEC15). The preparation can further include an adjuvant, such as Freund's complete or incomplete adjuvant, or a similar immunostimulatory agent.

Polyclonal antibodies can be prepared as described above by immunizing a suitable subject with a SIGLEC15 polypeptide, or an antigenic peptide thereof (e.g., part of SIGLEC15) as an immunogen. The antibody titer in the immunized subject can be monitored over time by standard techniques, such as with an enzyme-linked immunosorbent assay (ELISA) using the immobilized SIGLEC15 polypeptide or peptide. If desired, the antibody molecules can be isolated from the mammal (e.g., from the blood) and further purified by well-known techniques, such as protein A of protein G chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler et al. (Nature 256:495-497, 1975), the human B cell hybridoma technique (Kozbor et al., Immunol. Today 4:72, 1983), the EBV-hybridoma technique (Cole et al., Monoclonal Antibodies and Cancer Therapy, Alan R. Liss, Inc., pp. 77-96, 1985), or trioma techniques. The technology for producing hybridomas is well known (see, generally, Current Protocols in Immunology, 1994, Coligan et al. (Eds.), John Wiley & Sons, Inc., New York, NY). Hybridoma cells producing a monoclonal antibody are detected by screening the hybridoma culture supernatants for antibodies that bind the polypeptide or epitope of interest, e.g., using a standard ELISA assay.

Variants of the antibodies or antigen-binding fragments described herein can be prepared by introducing appropriate nucleotide changes into the DNA encoding a human, humanized, or chimeric antibody, or antigen-binding fragment thereof described herein, or by peptide synthesis. Such variants include, for example, deletions, insertions, or substitutions of residues within the amino acids sequences that make-up the antigen-binding site of the antibody or an antigen-binding domain. In a population of such variants, some antibodies or antigen-binding fragments will have increased affinity for the target protein, e.g., SIGLEC15. Any combination of deletions, insertions, and/or combinations can be made to arrive at an antibody or antigen-binding fragment thereof that has increased binding affinity for the target. The amino acid changes introduced into the antibody or antigen-binding fragment can also alter or introduce new post-translational modifications into the antibody or antigen-binding fragment, such as changing (e.g., increasing or decreasing) the number of glycosylation sites, changing the type of glycosylation site (e.g., changing the amino acid sequence such that a different sugar is attached by enzymes present in a cell), or introducing new glycosylation sites.

Antibodies disclosed herein can be derived from any species of animal, including mammals. Non-limiting examples of native antibodies include antibodies derived from humans, primates, e.g., monkeys and apes, cows, pigs, horses, sheep, camelids (e.g., camels and llamas), chicken, goats, and rodents (e.g., rats, mice, hamsters and rabbits), including transgenic rodents genetically engineered to produce human antibodies.

Human and humanized antibodies include antibodies having variable and constant regions derived from (or having the same amino acid sequence as those derived from) human germline immunoglobulin sequences. Human antibodies may include amino acid residues not encoded by human germline immunoglobulin sequences (e.g., mutations introduced by random or site-specific mutagenesis in vitro or by somatic mutation in vivo), for example in the CDRs.

A humanized antibody, typically has a human framework (FR) grafted with non-human CDRs. Thus, a humanized antibody has one or more amino acid sequence introduced into it from a source which is non-human. These non-human amino acid residues are often referred to as “import” residues, which are typically taken from an “import” variable domain. Humanization can be essentially performed by e.g., substituting rodent CDRs or CDR sequences for the corresponding sequences of a human antibody. These methods are described in e.g., Jones et al., Nature, 321:522-525 (1986); Riechmann et al., Nature, 332:323-327 (1988); Verhoeyen et al., Science, 239:1534-1536 (1988); each of which is incorporated by reference herein in its entirety. Accordingly, “humanized” antibodies are chimeric antibodies wherein substantially less than an intact human V domain has been substituted by the corresponding sequence from a non-human species. In practice, humanized antibodies are typically mouse antibodies in which some CDR residues and some FR residues are substituted by residues from analogous sites in human antibodies.

The choice of human VH and VL domains to be used in making the humanized antibodies is very important for reducing immunogenicity. According to the so-called “best-fit” method, the sequence of the V domain of a mouse antibody is screened against the entire library of known human-domain sequences. The human sequence which is closest to that of the mouse is then accepted as the human FR for the humanized antibody (Sims et al., J. Immunol., 151:2296 (1993); Chothia et al., J. Mol. Biol., 196:901 (1987)).

It is further important that antibodies be humanized with retention of high specificity and affinity for the antigen and other favorable biological properties. To achieve this goal, humanized antibodies can be prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved.

Ordinarily, amino acid sequence variants of the human, humanized, or chimeric anti-SIGLEC15 antibody will contain an amino acid sequence having at least 75%, 80%, 85%, 90%, 95%, 96%, 97%, 98%, or 99% percent identity with a sequence present in the light or heavy chain of the original antibody.

In some embodiments, a mouse (e.g., RenMab™ mouse) with a humanized heavy chain immunoglobulin locus and a humanized kappa chain immunoglobulin locus is used to generate antibodies. The heavy chain immunoglobulin locus is a region on the chromosome that contains genes for the heavy chains of antibodies. The locus can include e.g., human IGHV (variable) genes, human IGHD (diversity) genes, human IGHJ (joining) genes, and mouse heavy chain constant domain genes. The kappa chain immunoglobulin locus is a region on the chromosome that contains genes that encode the light chains of antibodies (kappa chain). The kappa chain immunoglobulin locus can include e.g., human IGKV (variable) genes, human IGKJ (joining) genes, and mouse light chain constant domain genes. A detailed description regarding RenMab™ mice can be found in PCT/CN2020/075698 or US20200390073A1, which is incorporated herein by reference in its entirety.

In some embodiments, a mouse (e.g., RenLite™ mouse) with a humanized heavy chain immunoglobulin locus and a humanized kappa chain immunoglobulin locus is used to generate antibodies. The heavy chain immunoglobulin locus is a region on the chromosome that contains genes for the heavy chains of antibodies. The locus can include e.g., human IGHV (variable) genes, human IGHD (diversity) genes, human IGHJ (joining) genes, and mouse heavy chain constant domain genes. The kappa chain immunoglobulin locus is a region on the chromosome that contains genes that encode a common light chains. The kappa chain immunoglobulin locus can include e.g., a human IGKV (variable) gene, a human IGKJ (joining) gene, and mouse light chain constant domain genes. A detailed description regarding RenLite™ mice can be found in PCT/CN2021/097652, which is incorporated herein by reference in its entirety.

The antibodies generated by the mice have a full human VH, a full human VL, and mouse constant regions. In some embodiments, the human VH and human VL is linked to a human IgG constant regions (e.g., IgG1, IgG2, IgG3, and IgG4). In some embodiments, the constant region has a sequence that is at least at least 80%, 85%, 90%, 91%, 92%, 93%, 94%, 95%, 96%, 97%, 98%, or 99% identical to one of SEQ ID NOs: 70-77.

Identity or homology with respect to an original sequence is usually the percentage of amino acid residues present within the candidate sequence that are identical with a sequence present within the human, humanized, or chimeric anti-SIGLEC15 antibody or fragment, after aligning the sequences and introducing gaps, if necessary, to achieve the maximum percent sequence identity, and not considering any conservative substitutions as part of the sequence identity.

Additional modifications to the anti-SIGLEC15 antibodies or antigen-binding fragments can be made. For example, a cysteine residue(s) can be introduced into the Fc region, thereby allowing interchain disulfide bond formation in this region. The homodimeric antibody thus generated may have any increased half-life in vitro and/or in vivo. Homodimeric antibodies with increased half-life in vitro and/or in vivo can also be prepared using heterobifunctional cross-linkers as described, for example, in Wolff et al. (Cancer Res. 53:2560-2565, 1993). Alternatively, an antibody can be engineered which has dual Fc regions (see, for example, Stevenson et al., Anti-Cancer Drug Design 3:219-230, 1989).

In some embodiments, a covalent modification can be made to the anti-SIGLEC15 antibody or antigen-binding fragment thereof. These covalent modifications can be made by chemical or enzymatic synthesis, or by enzymatic or chemical cleavage. Other types of covalent modifications of the antibody or antibody fragment are introduced into the molecule by reacting targeted amino acid residues of the antibody or fragment with an organic derivatization agent that is capable of reacting with selected side chains or the N- or C-terminal residues.

In some embodiments, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fe region residues; or position 314 in Kabat numbering); however, Asn297 may also be located about +3 amino acids upstream or downstream of position 297, i.e., between positions 294 and 300, due to minor sequence variations in antibodies. Such fucosylation variants may have improved ADCC function. In some embodiments, to reduce glycan heterogeneity, the Fc region of the antibody can be further engineered to replace the Asparagine at position 297 with Alanine (N297A).

In some embodiments, to facilitate production efficiency by avoiding Fab-arm exchange, the Fc region of the antibodies was further engineered to replace the serine at position 228 (EU numbering) of IgG4 with proline (S228P). A detailed description regarding S228 mutation is described, e.g., in Silva et al. “The S228P mutation prevents in vivo and in vitro IgG4 Fab-arm exchange as demonstrated using a combination of novel quantitative immunoassays and physiological matrix preparation.” Journal of Biological Chemistry 290.9 (2015): 5462-5469, which is incorporated by reference in its entirety.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant antibody polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

In some implementations, a polynucleotide disclosed herein (e.g., a polynucleotide that encodes a polypeptide disclosed herein) is introduced using a viral expression system (e.g., vaccinia or other pox virus, retrovirus, or adenovirus), which may involve the use of a non-pathogenic (defective), replication competent virus, or may use a replication defective virus. In the latter case, viral propagation generally will occur only in complementing virus packaging cells. Suitable systems are disclosed, for example, in Fisher-Hoch et al., 1989, Proc. Natl. Acad. Sci. USA 86:317-321; Flexner et al., 1989, Ann. N.Y. Acad Sci. 569:86-103; Flexner et al., 1990, Vaccine, 8:17-21; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner-Biotechniques, 6:616-627, 1988; Rosenfeld et al., 1991, Science, 252:431-434; Kolls et al., 1994, Proc. Natl. Acad. Sci. USA, 91:215-219; Kass-Eisler et al., 1993, Proc. Natl. Acad. Sci. USA, 90:11498-11502; Guzman et al., 1993, Circulation, 88:2838-2848; and Guzman et al., 1993, Cir. Res., 73:1202-1207. Techniques for incorporating DNA into such expression systems are well known to those of ordinary skill in the art. The DNA may also be “naked,” as described, for example, in Ulmer et al., 1993, Science, 259:1745-1749, and Cohen, 1993, Science, 259:1691-1692. The uptake of naked DNA may be increased by coating the DNA onto biodegradable beads that are efficiently transported into the cells.

For expression, the DNA insert comprising an antibody-encoding or polypeptide-encoding polynucleotide disclosed herein can be operatively linked to an appropriate promoter (e.g., a heterologous promoter), such as the phage lambda PL promoter, the E. coli lac, trp and tac promoters, the SV40 early and late promoters and promoters of retroviral LTRs, to name a few. Other suitable promoters are known to the skilled artisan. The expression constructs can further contain sites for transcription initiation, termination and, in the transcribed region, a ribosome binding site for translation. The coding portion of the mature transcripts expressed by the constructs may include a translation initiating at the beginning and a termination codon (UAA, UGA, or UAG) appropriately positioned at the end of the polypeptide to be translated.

As indicated, the expression vectors can include at least one selectable marker. Such markers include dihydrofolate reductase or neomycin resistance for eukaryotic cell culture and tetracycline or ampicillin resistance genes for culturing in E. coli and other bacteria. Representative examples of appropriate hosts include, but are not limited to, bacterial cells, such as E. coli. Streptomyces, and Salmonella typhimurium cells; fungal cells, such as yeast cells; insect cells such as Drosophila S2 and Spodoptera Sf9 cells; animal cells such as CHO, COS, Bowes melanoma, and HK 293 cells; and plant cells. Appropriate culture mediums and conditions for the host cells described herein are known in the art.

Non-limiting vectors for use in bacteria include pQE70, pQE60 and pQE-9, available from Qiagen; pBS vectors, Phagescript vectors, Bluescript vectors, pNH8A, pNH16a, pNH18A, pNH46A, available from Stratagene; and ptrc99a, pKK223-3, pKK233-3, pDR540, pRIT5 available from Pharmacia. Non-limiting eukaryotic vectors include pWLNEO, pSV2CAT, pOG44, pXT1 and pSG available from Stratagene; and pSVK3, pBPV, pMSG and pSVL available from Pharmacia. Other suitable vectors will be readily apparent to the skilled artisan.

Non-limiting bacterial promoters suitable for use include the E. coli lacl and lacZ promoters, the T3 and T7 promoters, the gpt promoter, the lambda PR and PL promoters and the trp promoter. Suitable eukaryotic promoters include the CMV immediate early promoter, the HSV thymidine kinase promoter, the early and late SV40 promoters, the promoters of retroviral LTRs, such as those of the Rous sarcoma virus (RSV), and metallothionein promoters, such as the mouse metallothionein-I promoter.

In the yeast Saccharomyces cerevisiae, a number of vectors containing constitutive or inducible promoters such as alpha factor, alcohol oxidase, and PGH may be used. For reviews, see Ausubel et al. (1989) Current Protocols in Molecular Biology, John Wiley & Sons, New York, N.Y, and Grant et al., Methods Enzymol., 153:516-544 (1997).

Introduction of the construct into the host cell can be effected by calcium phosphate transfection, DEAE-dextran mediated transfection, cationic lipid-mediated transfection, electroporation, transduction, infection or other methods. Such methods are described in many standard laboratory manuals, such as Davis et al., Basic Methods In Molecular Biology (1986), which is incorporated herein by reference in its entirety.

Transcription of DNA encoding an antibody of the present disclosure by higher eukaryotes may be increased by inserting an enhancer sequence into the vector. Enhancers are cis-acting elements of DNA, usually about from 10 to 300 bp that act to increase transcriptional activity of a promoter in a given host cell-type. Examples of enhancers include the SV40 enhancer, which is located on the late side of the replication origin at base pairs 100 to 270, the cytomegalovirus early promoter enhancer, the polyoma enhancer on the late side of the replication origin, and adenovirus enhancers.

For secretion of the translated protein into the lumen of the endoplasmic reticulum, into the periplasmic space or into the extracellular environment, appropriate secretion signals may be incorporated into the expressed polypeptide. The signals may be endogenous to the polypeptide or they may be heterologous signals.

The polypeptide (e.g., antibody) can be expressed in a modified form, such as a fusion protein (e.g., a GST-fusion) or with a histidine-tag, and may include not only secretion signals, but also additional heterologous functional regions. For instance, a region of additional amino acids, particularly charged amino acids, may be added to the N-terminus of the polypeptide to improve stability and persistence in the host cell, during purification, or during subsequent handling and storage. Also, peptide moieties can be added to the polypeptide to facilitate purification. Such regions can be removed prior to final preparation of the polypeptide. The addition of peptide moieties to polypeptides to engender secretion or excretion, to improve stability and to facilitate purification, among others, are familiar and routine techniques in the art.

### Methods of Treatment

The antibodies or antigen-binding fragments thereof of the present disclosure can be used for various therapeutic purposes.

In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount of an antibody or antigen-binding fragment thereof disclosed herein to a subject in need thereof (e.g., a subject having, or identified or diagnosed as having, a cancer), e.g., breast cancer (e.g., triple-negative breast cancer), carcinoid cancer, cervical cancer, endometrial cancer, glioma, head and neck cancer, liver cancer, lung cancer, small cell lung cancer, lymphoma, melanoma, ovarian cancer, pancreatic cancer, prostate cancer, renal cancer, colorectal cancer, gastric cancer, testicular cancer, thyroid cancer, bladder cancer, urethral cancer, or hematologic malignancy. In some embodiments, the cancer is unresectable melanoma or metastatic melanoma, non-small cell lung carcinoma (NSCLC), small cell lung cancer (SCLC), bladder cancer, or metastatic hormone-refractory prostate cancer. In some embodiments, the cancer is NSCLC, ovarian cancer, melanoma, colorectal cancer, breast cancer, a hematological malignancy, head and neck cancer, gastrointestinal cancer, bladder cancer, or bone cancer. In some embodiments, the subject has a solid tumor. In some embodiments, the cancer is squamous cell carcinoma of the head and neck (SCCHN), renal cell carcinoma (RCC), triple-negative breast cancer (TNBC), or colorectal carcinoma. In some embodiments, the subject has Hodgkin's lymphoma. In some embodiments, the subject has triple-negative breast cancer (TNBC), gastric cancer, urothelial cancer, Merkel-cell carcinoma, or head and neck cancer. In some embodiments, the cancer is melanoma, pancreatic carcinoma, mesothelioma, hematological malignancies, especially Non-Hodgkin's lymphoma, lymphoma, chronic lymphocytic leukemia, or advanced solid tumors.

In some embodiments, the compositions and methods disclosed herein can be used for treatment of patients at risk for a cancer. Patients with cancer can be identified with various methods known in the art.

In one aspect, the disclosure provides methods for treating, preventing, or reducing the risk of developing bone disorders, e.g., rickets, renal diseases (renal osteodystrophy, Fanconi syndrome), tumor-induced osteomalacia, hypophosphatasia, McCune-Albright syndrome, or osteogenesis imperfecta with mineralization defect (syndrome resembling osteogenesis imperfecta (SROI). In some embodiments, the disorder of bone mineralization is osteoporosis.

In one aspect, the disclosure provides methods for treating, preventing, or reducing the risk of developing disorders associated with an abnormal or unwanted immune response, e.g., an autoimmune disorder, e.g., by affecting the functional properties of the APC cells (e.g., by blocking the interaction between SIGLEC15 and SIGLEC15 ligands). These autoimmune disorders include, but are not limited to, Alopecia areata, lupus, ankylosing spondylitis, Meniere's disease, antiphospholipid syndrome, mixed connective tissue disease, autoimmune Addison's disease, multiple sclerosis, autoimmune hemolytic anemia, myasthenia gravis, autoimmune hepatitis, pemphigus vulgaris, Behcet's disease, pernicious anemia, bullous pemphigoid, polyarthritis nodosa, cardiomyopathy, polychondritis, celiac sprue-dermatitis, polyglandular syndromes, chronic fatigue syndrome (CFIDS), polymyalgia rheumatica, chronic inflammatory demyelinating, polymyositis and dermatomyositis, chronic inflammatory polyneuropathy, primary agammaglobulinemia, Churg-Strauss syndrome, primary biliary cirrhosis, cicatricial pemphigoid, psoriasis, CREST syndrome, Raynaud's phenomenon, cold agglutinin disease, Reiter's syndrome, Crohn's disease, Rheumatic fever, discoid lupus, rheumatoid arthritis, Cryoglobulinemia sarcoidosis, fibromyalgia, scleroderma, Grave's disease, Sjögren's syndrome, Guillain-Barre, stiff-man syndrome, Hashimoto's thyroiditis, Takayasu arteritis, idiopathic pulmonary fibrosis, temporal arteritis/giant cell arteritis, idiopathic thrombocytopenia purpura (ITP), ulcerative colitis, IgA nephropathy, uveitis, diabetes (e.g., Type I), vasculitis, lichen planus, and vitiligo. The anti-SIGLEC15 antibodies or antigen-binding fragments thereof can also be administered to a subject to treat, prevent, or reduce the risk of developing disorders associated with an abnormal or unwanted immune response associated with cell, tissue or organ transplantation, e.g., renal, hepatic, and cardiac transplantation, e.g., graft versus host disease (GVHD), or to prevent allograft rejection. In some embodiments, the subject has dermatological disorders, liver disease (e.g., cirrhosis), Hidradenitis, experimental autoimmune encephalomyelitis. In some embodiments, the subject has renal disease, lupus, Sjogren's syndrome, ulcerative colitics, psoriasis, Hidradenitis suppurativa, Immune Thrombocytopenia (ITP), or other inflammatory arthritis. In some embodiments, the subject has multiple sclerosis or myasthenia gravis. In some embodiments, the subject has Crohn's disease, ulcerative colitis or type 1 diabetes. In some embodiments, the subject has autoimmune thyroid disease, Grave's disease, multiple sclerosis, psoriasis, inflammatory bowel disease (e.g., Crohn's Disease (CD) and ulcerative colitis), rheumatoid arthritis, Sjögren's syndrome, autoimmune nephritis, or systemic lupus erythematosus. In some embodiments, the methods involve administering to the subject an effective amount of a composition comprising the antibody or antigen-binding fragment thereof as described herein.

As used herein, by an “effective amount” is meant an amount or dosage sufficient to effect beneficial or desired results including halting, slowing, retarding, or inhibiting progression of a disease, e.g., an autoimmune disease or a cancer. An effective amount will vary depending upon, e.g., an age and a body weight of a subject to which the antibody, antigen binding fragment, antibody-encoding polynucleotide, vector comprising the polynucleotide, and/or compositions thereof is to be administered, a severity of symptoms and a route of administration, and thus administration can be determined on an individual basis.

An effective amount can be administered in one or more administrations. By way of example, an effective amount of an antibody or an antigen binding fragment is an amount sufficient to ameliorate, stop, stabilize, reverse, inhibit, slow and/or delay progression of an autoimmune disease or a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective amount of an antibody or antigen binding fragment may vary, depending on, inter alia, patient history as well as other factors such as the type (and/or dosage) of antibody used.

Effective amounts and schedules for administering the antibodies, antibody-encoding polynucleotides, and/or compositions disclosed herein may be determined empirically, and making such determinations is within the skill in the art. Those skilled in the art will understand that the dosage that must be administered will vary depending on, for example, the mammal that will receive the antibodies, antibody-encoding polynucleotides, and/or compositions disclosed herein, the route of administration, the particular type of antibodies, antibody-encoding polynucleotides, antigen binding fragments, and/or compositions disclosed herein used and other drugs being administered to the mammal. Guidance in selecting appropriate doses for antibody or antigen binding fragment can be found in the literature on therapeutic uses of antibodies and antigen binding fragments, e.g., Handbook of Monoclonal Antibodies, Ferrone et al., eds., Noges Publications, Park Ridge, N.J., 1985, ch. 22 and pp. 303-357; Smith et al., Antibodies in Human Diagnosis and Therapy, Haber et al., eds., Raven Press, New York, 1977, pp. 365-389.

A typical daily dosage of an effective amount of an antibody is 0.01 mg/kg to 100 mg/kg. In some embodiments, the dosage can be less than 100 mg/kg, 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, or 0.1 mg/kg. In some embodiments, the dosage can be greater than 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.5 mg/kg, 0.1 mg/kg, 0.05 mg/kg, or 0.01 mg/kg. In some embodiments, the dosage is about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg.

In any of the methods described herein, the at least one antibody, antigen-binding fragment thereof, or pharmaceutical composition (e.g., any of the antibodies, antigen-binding fragments, or pharmaceutical compositions described herein) and, optionally, at least one additional therapeutic agent can be administered to the subject at least once a week (e.g., once a week, twice a week, three times a week, four times a week, once a day, twice a day, or three times a day). In some embodiments, at least two different antibodies and/or antigen-binding fragments are administered in the same composition (e.g., a liquid composition). In some embodiments, at least one antibody or antigen-binding fragment and at least one additional therapeutic agent are administered in the same composition (e.g., a liquid composition). In some embodiments, the at least one antibody or antigen-binding fragment and the at least one additional therapeutic agent are administered in two different compositions (e.g., a liquid composition containing at least one antibody or antigen-binding fragment and a solid oral composition containing at least one additional therapeutic agent). In some embodiments, the at least one additional therapeutic agent is administered as a pill, tablet, or capsule. In some embodiments, the at least one additional therapeutic agent is administered in a sustained-release oral formulation.

In some embodiments, the one or more additional therapeutic agents can be administered to the subject prior to, or after administering the at least one antibody, antigen-binding antibody fragment, or pharmaceutical composition (e.g., any of the antibodies, antigen-binding antibody fragments, or pharmaceutical compositions described herein). In some embodiments, the one or more additional therapeutic agents and the at least one antibody, antigen-binding antibody fragment, or pharmaceutical composition (e.g., any of the antibodies, antigen-binding antibody fragments, or pharmaceutical compositions described herein) are administered to the subject such that there is an overlap in the bioactive period of the one or more additional therapeutic agents and the at least one antibody or antigen-binding fragment (e.g., any of the antibodies or antigen-binding fragments described herein) in the subject.

In some embodiments, the subject can be administered the at least one antibody, antigen-binding antibody fragment, or pharmaceutical composition (e.g., any of the antibodies, antigen-binding antibody fragments, or pharmaceutical compositions described herein) over an extended period of time (e.g., over a period of at least 1 week, 2 weeks, 3 weeks, 1 month, 2 months, 3 months, 4 months, 5 months, 6 months, 7 months, 8 months, 9 months, 10 months, 11 months, 12 months, 1 year, 2 years, 3 years, 4 years, or 5 years). A skilled medical professional may determine the length of the treatment period using any of the methods described herein for diagnosing or following the effectiveness of treatment (e.g., the observation of at least one symptom of cancer). As described herein, a skilled medical professional can also change the identity and number (e.g., increase or decrease) of antibodies or antigen-binding antibody fragments (and/or one or more additional therapeutic agents) administered to the subject and can also adjust (e.g., increase or decrease) the dosage or frequency of administration of at least one antibody or antigen-binding antibody fragment (and/or one or more additional therapeutic agents) to the subject based on an assessment of the effectiveness of the treatment (e.g., using any of the methods described herein and known in the art).

In some embodiments, one or more additional therapeutic agents can be administered to the subject. The additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat).

In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAMI agonist, and a Selectin agonist.

In some embodiments, carboplatin, nab-paclitaxel, paclitaxel, cisplatin, pemetrexed, gemcitabine, FOLFOX, or FOLFIRI are administered to the subject.

In some embodiments, the additional therapeutic agent is an anti-OX40 antibody, an anti-PD-1 antibody, an anti-PD-L1 antibody, an anti-PD-L2 antibody, an anti-LAG-3 antibody, an anti-TIGIT antibody, an anti-BTLA antibody, an anti-CTLA-4 antibody, anti-ICOS antibody, anti-CD27 antibody, anti-OX40 antibody, anti-4-1BB antibody, and/or an anti-GITR antibody.

In one aspect, the disclosure provides a combination therapy. In some embodiments, the anti-SIGLEC15 antibody or antigen-binding fragment thereof (e.g., any antibody described herein) can be administered together with an anti-PD-L1 antibody. In some embodiments, the anti-SIGLEC15 antibody or antigen-binding fragment thereof (e.g., any antibody described herein) can be administered together with an anti-OX40 antibody. In some embodiments, the anti-SIGLEC15 antibody or antigen-binding fragment thereof (e.g., any antibody described herein) can be administered together with an anti-4-1-BB antibody.

### Pharmaceutical Compositions and Routes of Administration

Also provided herein are pharmaceutical compositions that contain at least one (e.g., one, two, three, or four) of the antibodies or antigen-binding fragments described herein. Two or more (e.g., two, three, or four) of any of the antibodies or antigen-binding fragments described herein can be present in a pharmaceutical composition in any combination. The pharmaceutical compositions may be formulated in any manner known in the art.

Pharmaceutical compositions are formulated to be compatible with their intended route of administration (e.g., intravenous, intraarterial, intramuscular, intradermal, subcutaneous, or intraperitoneal). The compositions can include a sterile diluent (e.g., sterile water or saline), a fixed oil, polyethylene glycol, glycerine, propylene glycol or other synthetic solvents, antibacterial or antifungal agents, such as benzyl alcohol or methyl parabens, chlorobutanol, phenol, ascorbic acid, thimerosal, and the like, antioxidants, such as ascorbic acid or sodium bisulfite, chelating agents, such as ethylenediaminetetraacetic acid, buffers, such as acetates, citrates, or phosphates, and isotonic agents, such as sugars (e.g., dextrose), polyalcohols (e.g., mannitol or sorbitol), or salts (e.g., sodium chloride), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating, such as lecithin, or a surfactant. Absorption of the antibody or antigen-binding fragment thereof can be prolonged by including an agent that delays absorption (e.g., aluminum monostearate and gelatin). Alternatively, controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid; Alza Corporation and Nova Pharmaceutical, Inc.).

Compositions containing one or more of any of the antibodies or antigen-binding fragments described herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions for parenteral administration are preferably sterile and substantially isotonic and manufactured under Good Manufacturing Practice (GMP) conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, preferably in physiologically-compatible buffers to reduce discomfort at the site of injection. The solution can contain formulatory agents such as suspending, stabilizing and/or dispersing agents. Alternatively antibodies can be in lyophilized form for constitution with a suitable vehicle, e.g., sterile pyrogen-free water, before use.

Toxicity and therapeutic efficacy of compositions can be determined by standard pharmaceutical procedures in cell cultures or experimental animals (e.g., monkeys). One can, for example, determine the LD50 (the dose lethal to 50% of the population) and the ED50 (the dose therapeutically effective in 50% of the population): the therapeutic index being the ratio of LD50:ED50. Agents that exhibit high therapeutic indices are preferred. Where an agent exhibits an undesirable side effect, care should be taken to minimize potential damage (i.e., reduce unwanted side effects). Toxicity and therapeutic efficacy can be determined by other standard pharmaceutical procedures.

Data obtained from cell culture assays and animal studies can be used in formulating an appropriate dosage of any given agent for use in a subject (e.g., a human). A therapeutically effective amount of the one or more (e.g., one, two, three, or four) antibodies or antigen-binding fragments thereof (e.g., any of the antibodies or antibody fragments described herein) will be an amount that treats the disease in a subject (e.g., kills cancer cells) in a subject (e.g., a human subject identified as having cancer), or a subject identified as being at risk of developing the disease (e.g., a subject who has previously developed cancer but now has been cured), decreases the severity, frequency, and/or duration of one or more symptoms of a disease in a subject (e.g., a human). The effectiveness and dosing of any of the antibodies or antigen-binding fragments described herein can be determined by a health care professional or veterinary professional using methods known in the art, as well as by the observation of one or more symptoms of disease in a subject (e.g., a human). Certain factors may influence the dosage and timing required to effectively treat a subject (e.g., the severity of the disease or disorder, previous treatments, the general health and/or age of the subject, and the presence of other diseases).

Exemplary doses include milligram or microgram amounts of any of the antibodies or antigen-binding fragments described herein per kilogram of the subject's weight (e.g., about 1 μg/kg to about 500 mg/kg; about 100 μg/kg to about 500 mg/kg; about 100 μg/kg to about 50 mg/kg; about 10 μg/kg to about 5 mg/kg; about 10 μg/kg to about 0.5 mg/kg; or about 1 μg/kg to about 50 μg/kg). While these doses cover a broad range, one of ordinary skill in the art will understand that therapeutic agents, including antibodies and antigen-binding fragments thereof, vary in their potency, and effective amounts can be determined by methods known in the art. Typically, relatively low doses are administered at first, and the attending health care professional or veterinary professional (in the case of therapeutic application) or a researcher (when still working at the development stage) can subsequently and gradually increase the dose until an appropriate response is obtained. In addition, it is understood that the specific dose level for any particular subject will depend upon a variety of factors including the activity of the specific compound employed, the age, body weight, general health, gender, and diet of the subject, the time of administration, the route of administration, the rate of excretion, and the half-life of the antibody or antibody fragment in vivo.

The pharmaceutical compositions can be included in a container, pack, or dispenser together with instructions for administration. The disclosure also provides methods of manufacturing the antibodies or antigen binding fragments thereof for various uses as described herein.

### EXAMPLES

The invention is further described in the following examples, which do not limit the scope of the invention described in the claims.

### Example 1. Generating Human Anti-SIGLEC15 Antibodies

To generate antibodies against human SIGLEC15, RenMab™ mice were immunized with human SIGLEC15. Anti-SIGLEC15 antibodies were made by the methods as described below.

RenMab™ mice have both a humanized heavy chain immunoglobulin locus and a humanized kappa chain immunoglobulin locus. The heavy chain immunoglobulin locus is a region on the chromosome that contains genes for the heavy chains of antibodies. The locus includes IGHV (variable), IGHD (diversity), IGHJ (joining), and heavy chain constant domain genes. The kappa chain immunoglobulin locus is a region on the chromosome that contains genes that encode the light chains of antibodies (kappa chain). The kappa chain immunoglobulin locus includes IGKV (variable), IGKJ (joining), and light chain constant domain genes. Detailed descriptions regarding RenMab™ mice can be found in PCT/CN2020/075698, which is incorporated herein by reference in its entirety.

### Immunization of Mice

Human SIGLEC15 protein (hSIGLEC15-Fc, ACRO Biosystems Inc., Cat #: SG5-H5253, including positions 20-263 of SEQ ID NO: 45) and mouse SIGLEC15 protein (mSIGLEC15-His, ACRO Biosystems Inc., Cat #: SG5-M52H7, including positions 24-262 of SEQ ID NO: 46) were emulsified with adjuvants, and was used to immunize RenMab™ mice. Before immunization, retro-orbital blood was collected as a negative control.

Freund's complete adjuvant CFA was used for the first immunization and Freund's incomplete adjuvant IFA was used for the second, third, and fourth immunizations. A total of four immunizations were performed. The first and second immunizations were separated by two weeks, and the remaining immunizations were separated by one week. One week after the fourth immunization, retro-orbital blood was collected, and the antibody titer of serum was detected by FACS.

In another experiment, several mice are immunized by injecting the expression plasmids encoding human SIGLEC15 or mouse SIGLEC15 into the mice (SIGLEC15 KO mice). The plasmids encoding the antigen are injected into the tibialis anterior muscle (intramuscular injection; i.m. injection). At least four injections are performed with at least 14 days between two injections. Blood (serum) is collected seven days after the last immunization and the serum is tested for antibody titer by FACS.

Procedures to enhance immunization were also performed at least fourteen days after the previous immunization (either by injecting the proteins or by injecting the plasmids). SIGLEC15 protein was injected by intraperitoneal injection, and the CHO-S cells expressing human SIGLEC15 antigen was injected through the tail vein.

Antigen-specific immune cells were isolated from the immunized mice to further obtain anti-SIGLEC15 antibodies or to obtain the light chain and heavy chain variable region sequences of the anti-SIGLEC15 antibodies. For example, single cell technology (e.g., using Beacon® Optofluidic System, Berkeley Lights Inc.) was used to screen and find plasma cells that secrete antigen-specific monoclonal antibodies, and reverse transcription and PCR sequencing were used to obtain antibody variable region sequences. The obtained variable region sequences were used for antibody expression to verify the binding affinity to SIGLEC15 using FACS. Specifically, the obtained VH and VL sequences were respectively connected to a human IgG1 constant region that include the N297A mutation. Exemplary antibodies obtained by this method included: 10A7, 10C9 and 23F4.

The amino acid sequences of heavy chain CDRs 1, 2, 3, and light chain CDRs 1, 2, 3 for 10A7 are shown in SEQ ID NOs: 1-6 (Kabat numbering) or SEQ ID NOs: 19-24 (Chothia numbering), respectively. The human heavy chain variable region and human light chain variable region for 10A7 are shown in SEQ ID NO: 37 and SEQ ID NO: 38, respectively.

The amino acid sequences of heavy chain CDRs 1, 2, 3, and light chain CDRs 1, 2, 3 for 10C9 are shown in SEQ ID NOs: 7-12 (Kabat numbering) or SEQ ID NOs: 25-30 (Chothia numbering), respectively. The human heavy chain variable region and human light chain variable region for 10C9 are shown in SEQ ID NO: 39 and SEQ ID NO: 40, respectively.

The amino acid sequences of heavy chain CDRs 1, 2, 3, and light chain CDRs 1, 2, 3 for 23F4 are shown in SEQ ID NOs: 13-18 (Kabat numbering) or SEQ ID NOs: 31-36 (Chothia numbering), respectively. The human heavy chain variable region and human light chain variable region for 23F4 are shown in SEQ ID NO: 41 and SEQ ID NO: 42, respectively.

### Construction of One-Armed Antibodies

Further, one-armed antibodies were constructed, the antibody has an anti-SIGLEC15 arm comprising a heavy chain and a light chain, and a heavy chain fragment comprising CH2 and CH3 domains of IgG (FIG. 1B).

Vectors expressing respective polypeptide chains of the one-armed antibody were co-transfected into CHO cells. After 14 days of culture, the cell supernatant was collected and purified by Protein A affinity chromatography. Constant domains of the antibody were selected from either human IgG1 or IgG4. In particular, mutations within IgG1, e.g., N297A mutations, were also introduced to reduce Fc receptor binding affinities. These Fc mutations can improve antibody safety by minimizing antibody effector functions. Exemplary antibodies obtained by this method included: 10C9-S and 23F4-S.

In the one-armed antibodies, knobs-into-holes (KIH) mutations were also introduced to the constant regions. The knobs-into-holes technology was employed to construct a one-armed anti-SIGLEC15 antibody by introducing a knob mutation (T366W) in the Fc of a full-length heavy chain and hole mutations (T366S, L368A, and Y407V) in the single Fc fragment (FIG. 1). The one-armed anti-SIGLEC15 antibody 10C9-S, human IgG1-N297A was generated (the full-length heavy chain with knob SEQ ID NO: 43, the single Fc fragment SEQ ID NO: 44, the full-length light chain SEQ ID NO: 78)

### Example 2. Cross-Species Binding of Anti-SIGLEC15 Antibodies

CHO-S-hSIGLEC15 cells, CHO-S-mSIGLEC15 cells, CHO-S-fasSIGLEC15 cells or CHO-S-dSIGLEC15 cells were transferred to a 96-well plate at a density of 5×10⁴ cells/well respectively. Serially diluted sample anti-SIGLEC15 antibodies were added to the 96-well plate, and incubated at 4° C. for 30 min. Then, the cells were incubated with the secondary antibody anti-hIgG-Fc-Alex Flour 647 (RL1-H) (Jackson ImmunoResearch Laboratories, Inc., 109-606-170) at 4° C. in the dark for 15 minutes before flow cytometry analysis.

CHO-S-hSIGLEC15 cells, CHO-S-mSIGLEC15 cells. CHO-S-fasSIGLEC15 cells or CHO-S-dSIGLEC15 cells were obtained by transfecting CHO-S cells with human SIGLEC15 amino acid sequence (hSIGLEC15, SEQ ID NO: 45), mouse SIGLEC15 amino acid sequence (mSIGLEC15, SEQ ID NO: 46), positions 60-387 of Macaca fascicularis (crab-eating macaque) SIGLEC15 amino acid sequence (fasSIGLEC15, SEQ ID NO: 47) and dog SIGLEC15 amino acid sequence (dSIGLEC15, SEQ ID NO: 48), respectively.

The test results are shown in the table below. All anti-SIGLEC15 antibodies 10A7, 10C9 and 23F4 can bind to hSIGLEC15, mSIGLEC15, fasSIGLEC15 and dSIGLEC15.

TABLE 1

| | | Percentage of | |
|---|---|---|---|
| Antibody | SIGLEC15 Protein | Positive Cells | Evaluation |
| NC | hSIGLEC15 | 0.1% | No Binding |
| | mSIGLEC15 | 0.1% | No Binding |
| | fasSIGLEC15 | 0.2% | No Binding |
| | dSIGLEC15 | 0.6% | No Binding |
| NC + | hSIGLEC15 | 0 | No Binding |
| secondary | mSIGLEC15 | 0.3% | No Binding |
| antibody | fasSIGLEC15 | 0 | No Binding |
| | dSIGLEC15 | 0.7% | No Binding |
| 10A7 | hSIGLEC15 | 17.9% | Binding |
| | mSIGLEC15 | 38.5% | Binding |
| | fasSIGLEC15 | 49.3% | Binding |
| | dSIGLEC15 | 22.8% | Binding |
| 10C9 | hSIGLEC15 | 16.3% | Binding |
| | mSIGLEC15 | 29.3% | Binding |
| | fasSIGLEC15 | 47.5% | Binding |
| | dSIGLEC15 | 37.3% | Binding |
| 23F4 | hSIGLEC15 | 25.6% | Binding |
| | mSIGLEC15 | 40.5% | Binding |
| | fasSIGLEC15 | 50.0% | Binding |
| | dSIGLEC15 | 24.6% | Binding |

Another similar experiment was performed to determine whether the anti-SIGLEC15 antibodies can bind with other SIGLEC family proteins. CHO-S-hSIGLEC1 cells (expressing hSIGLEC1, SEQ ID NO: 49)), CHO-S-hSIGLEC2 cells (expressing hSIGLEC2, SEQ ID NO: 50), CHO-S-hSIGLEC4 cells (expressing hSIGLEC4, SEQ ID NO: 51), CHO-S-hSIGLEC5 cells (expressing hSIGLEC5, SEQ ID NO: 52), CHO-S-hSIGLEC6 cells (expressing hSIGLEC6, SEQ ID NO: 53), CHO-S-hSIGLEC7 cells (expressing hSIGLEC7, SEQ ID NO: 54), CHO-S-hSIGLEC8 cells (expressing hSIGLEC8, SEQ ID NO: 55), CHO-S-hSIGLEC9 cells (expressing hSIGLEC9, SEQ ID NO: 56), CHO-S-hSIGLEC10 cells (expressing hSIGLEC10, SEQ ID NO: 57), CHO-S-hSIGLEC11 cells (expressing hSIGLEC11, SEQ ID NO: 58), CHO-S-hSIGLEC12 cells (expressing hSIGLEC12, SEQ ID NO: 59), CHO-S-hSIGLEC14 cells (expressing hSIGLEC14, SEQ ID NO: 60) or CHO-S-hSIGLEC16 cells (expressing hSIGLEC16, SEQ ID NO: 61) were used.

The results are shown in the table below. Anti-SIGLEC15 antibodies 10A7, 10C9 and 23F4 don't bind to the other SIGLEC family proteins.

TABLE 2

| | SIGLEC family | Percentage of | |
|---|---|---|---|
| Antibody | protein | Positive Cells | Evaluation |
| 10A7 | hSIGLEC1 | 0 | No Binding |
| | hSIGLEC2 | 0 | No Binding |
| | hSIGLEC4 | 1.1% | No Binding |
| | hSIGLEC5 | 0.2% | No Binding |
| | hSIGLEC6 | 0.7% | No Binding |
| | hSIGLEC7 | 0.2% | No Binding |
| | hSIGLEC8 | 0.1% | No Binding |
| | hSIGLEC9 | 0.3% | No Binding |
| | hSIGLEC10 | 1.4% | No Binding |
| | hSIGLEC11 | 0.1% | No Binding |
| | hSIGLEC12 | 0 | No Binding |
| | hSIGLEC14 | 0.1% | No Binding |
| | hSIGLEC16 | 0.2% | No Binding |
| 10C9 | hSIGLEC1 | 0.1% | No Binding |
| | hSIGLEC2 | 0.3% | No Binding |
| | hSIGLEC4 | 1.2% | No Binding |
| | hSIGLEC5 | 0.2% | No Binding |
| | hSIGLEC6 | 0.5% | No Binding |
| | hSIGLEC7 | 0.4% | No Binding |
| | hSIGLEC8 | 0.3% | No Binding |
| | hSIGLEC9 | 0.1% | No Binding |
| | hSIGLEC10 | 1.5% | No Binding |
| | hSIGLEC11 | 0.1% | No Binding |
| | hSIGLEC12 | 0.1% | No Binding |
| | hSIGLEC14 | 0.2% | No Binding |
| | hSIGLEC16 | 0.2% | No Binding |
| 23F4 | hSIGLEC1 | 1.1% | No Binding |
| | hSIGLEC2 | 1.8% | No Binding |
| | hSIGLEC4 | 0.8% | No Binding |
| | hSIGLEC5 | 7.0% | No Binding |
| | hSIGLEC6 | 1.5% | No Binding |
| | hSIGLEC7 | 1.0% | No Binding |
| | hSIGLEC8 | 0.6% | No Binding |
| | hSIGLEC9 | 0.7% | No Binding |
| | hSIGLEC10 | 1.6% | No Binding |
| | hSIGLEC11 | 0.2% | No Binding |
| | hSIGLEC12 | 0.4% | No Binding |
| | hSIGLEC14 | 1.3% | No Binding |
| | hSIGLEC16 | 1.3% | No Binding |

### Example 3. Binding Affinity of Anti-SIGLEC15 Antibodies

The binding affinity of the anti-SIGLEC15 antibodies to His-tagged SIGLEC15 protein of human (hSIGLEC15-His, Novoprotein Scientific Inc., Cat #: CW37) (positions 20-263 of SEQ ID NO: 45), mouse (mSIGLEC15-His, ACROBiosystems Inc., Cat #: SG5-M52H7) (positions 24-262 of SEQ ID NO: 46) or monkey (fasSIGLEC15-His, ACROBiosystems Inc., Cat #: SG5-C52H6) (SEQ ID NO: 62) were verified using surface plasmon resonance (SPR) using Biacore™ (Biacore, Inc., Piscataway N.J.) 8K biosensor equipped with pre-immobilized Protein A sensor chips.

Purified anti-SIGLEC15 antibodies was captured on the Protein A chip (Series S Sensor Chip Protein A) for the detection. 1 μg/mL Purified anti-SIGLEC15 antibodies was loaded at 10 μL/min to bind to a gradient concentration of recombinant hSIGLEC15, fasSIGLEC15 and mSIGELC15 (50, 25, 12.5, 6.25, 3.125, 1.5626, 0.78125 and 0 nM). The flow rate was 30 μL/min, the binding and dissociation time were set to 180 s and 600 s, respectively. The chip was regenerated after the last injection of each titration with a glycine solution (pH 2.0) at 30 μL/min for 30 seconds.

Kinetic association rates (kon) and dissociation rates (koff) were obtained simultaneously by fitting the data globally to a 1:1 Langmuir binding model (Karlsson, R. Roos, H. Fagerstam, L. Petersson, B., 1994. Methods Enzymology 6. 99-110) using Biacore™ 8K Evaluation Software 3.0. Affinities were deduced from the quotient of the kinetic rate constants (KD=koff/kon). As a person of ordinary skill in the art would understand, the same method with appropriate adjustments for parameters (e.g., antibody concentration) was performed for each tested antibody. The results for the tested antibodies are summarized in the table below.

TABLE 3

| Protein | Analyte 1 Solution | kon (1/Ms) | koff (1/s) | KD (M) |
|---|---|---|---|---|
| 5G12 | hSIGLEC15-His | 2.93E+06 | 8.56E−04 | 2.92E−10 |
| | fasSIGLEC15-His | 2.62E+06 | 7.50E−04 | 2.86E−10 |
| | mSIGLEC15-His | 2.85E+06 | 1.18E−03 | 4.13E−10 |
| 10C9 | hSIGLEC15-His | 2.63E+06 | 2.23E−07 | 8.46E−14 |
| | fasSIGLEC15-His | 2.43E+06 | 5.01E−05 | 2.06E−11 |
| | mSIGLEC15-His | 1.17E+06 | 2.53E−03 | 2.17E−09 |
| 23F4 | hSIGLEC15-His | 4.19E+06 | 6.85E−07 | 1.64E−13 |
| | fasSIGLEC15-His | 8.22E+05 | 1.67E−04 | 2.04E−10 |
| | mSIGLEC15-His | 6.39E+05 | 1.26E−03 | 1.97E−09 |

5G12 is a humanized IgG1 monoclonal antibody targeting human SIGLEC15 in phase I/II clinical development at NextCure for the treatment of patients with locally advanced or metastatic solid tumors. The heavy chain sequence of the 5G12 antibody containing the N297A mutation is shown in SEQ ID NO: 63. The light chain sequence of the 5G12 antibody is shown in SEQ ID NO: 64.

The results show that the anti-SIGLEC15 antibodies 10C9 and 23F4 can all bind to human and monkey SIGLEC15 with high affinity.

### Example 4. Epitope Analysis of Anti-SIGLEC15 Antibodies

Relative positions of target protein epitope between a pair of purified anti-SIGLEC15 antibodies were analyzed by Biolayer Interferometry (BLI) using ForteBio Octet system at 30° C. A total of 4 purified antibodies were used: 5G12, 10A7, 10C9 and 23F4.

1×HBS-EP+ buffer (10 mM 4-(2-hydroxyethyl)-1-piperazineethanesulfonic acid (HEPES), 150 mM NaCl, 3 mM ethylenediaminetetraacetic acid (EDTA) and 0.05% P20, pH7.4) diluted from HBS-EP+ buffer (10×) was used as the running buffer throughout the experiment. About 100 RU of hSIGLEC15 protein was captured at a flow rate of 10 μL/min, and 200 nM of antibody was injected at a flow rate of 30 μL/min to bind the ligand. Another antibody was injected under the same conditions to determine whether the binding of different antibodies interfered with each other. The binding time was 300 s for each antibody.

The binding value of each antibody was obtained using Data Analysis HT 12.0. To quantify the interference of one antibody binding to another, a binding ratio was calculated to compare each pair of antibodies. The binding ratio is defined as the binding value of the second antibody (analyte 2), divided by the binding value of the first antibody (analyte 1). The binding ratio of each antibody pair was summarized in a matrix table as shown in Table 4. More specifically, the binding ratio was between-0.1 to 0.5, if analyte 1 exhibited a blocking effect to analyte 2. The binding ratio was between 0.5-1.2, if analyte 1 did not exhibit a blocking effect to analyte 2. In general, antibody pairs that interfere with each other have the same or overlapping epitopes.

The epitope binding assay results show that 23F4 and 10C9 recognize different epitopes, 10A7, 10C9 and 5G12 exhibited a strong correlation with each other.

TABLE 4

| Antibody | 5G12 | 10A7 | 10C9 | 23F4 |
|---|---|---|---|---|
| 5G12 | 0.08 | −0.08 | −0.05 | 0.51 |
| 10A7 | 0.20 | 0.14 | 0.16 | 0.43 |
| 10C9 | 0.23 | 0.11 | 0.11 | 0.72 |
| 23F4 | 0.48 | 0.18 | 0.62 | 0.07 |

### Example 5. Stability Analysis of Anti-SIGLEC15 Antibodies

Anti-SIGLEC15 antibodies 10C9 and 23F4 were diluted to 2 mg/mL using a buffer at pH 6.0 (3 mg/mL histidine, 80 mg/mL sucrose, and 0.2 mg/mL Tween 80). The diluted antibodies were kept in sealed Eppendorf tubes at 5±3° C. (hereinafter referred to as 4° C.) for 7 days; 25±2° C. (hereinafter referred to as 25° C.) for 7 days; or at 40±2° C. (hereinafter referred to as 40° C.) for 7 days, and their thermal stability was evaluated.

Freeze-thaw stability was determined with the below experiments: “freeze 1”: freezing at −80° C. for 5 days and then thawing at 4° C. for detection; “freeze 10”: freezing at −80° C., repeat freeze-thaw 10 times within 5 days (freezing at −80° C. and thawing at 4° C.);

Low pH stability: the stability index of the antibody was determined before and after six hours of incubation in 1 mol/L acetic acid, pH 3.5.

Specifically, the following tests were performed: (1) observing the solution appearance and presence of visible non-soluble objects; (2) detecting the purity changes of antibodies by Size-Exclusion Ultra Performance Liquid Chromatography (SEC-UPLC) (indicated as the percentage of the main peak area to the sum of all peak areas (Purity, %)); (3) detecting changes in the apparent hydrophobicity of the antibodies using the Hydrophobic Interaction Chromatography-High Performance Liquid Chromatography (HIC-HPLC) method (indicated as the retention time of the main peak (HIC, min); (4) detecting charge variants in the antibodies by Capillary Isoelectric Focusing (cIEF) (indicated as the percentages of the main component, acidic component, and alkaline component); (5) detecting antibody activity using flow cytometry (EC50).

In the SEC-UPLC experiments, the Agilent 1290 chromatograph system (connected with XBridge™ Protein BEH SEC column (200 Å, Waters Corporation)) was used. The antibody samples were diluted to 1 mg/mL with purified water. The following parameters were used: mobile phase: 100 mmol/L phosphate buffer (PB) (pH 7.4)+0.2 mol/L NaCl+10% acetonitrile; flow rate: 1.8 mL/min; column temperature: 25° C.; detection wavelength: 280 nm; injection volume: 10 μg; sample tray temperature: 6° C.; and running time: 7 minutes.

In the HIC-HPLC experiments, the Agilent 1260 chromatograph system (connected with ProPac™ HIC-10 column (4.6×250 mm, Thermo Scientific)) was used, and samples were diluted using mobile phase A to 0.5 mg/mL. The following parameters were used: mobile phase A: 0.9 M ammonium sulfate, 0.1 M PB, 10% acetonitrile pH 6.5; mobile phase B: 0.1 M PB, 10% acetonitrile pH 6.5; flow rate: 0.8 mL/min; gradient: 0 min 100% A, 2 min 100% A, 32 min 100% B, 34 min 100% B, 35 min 100% A, and 45 min 100% A; column temperature: 30° C.; detection wavelength: 280 nm; injection volume: 10 μg; sample tray temperature: about 6° C.; and running time: 45 minutes.

In the cIEF experiments, the Maurice cIEF Method Development Kit (Protein Simple, Cat #: PS-MDK01-C) was used for sample preparation. Specifically, 40 μg protein sample was mixed with the following reagents in the kit: 1 μL Maurice cIEF pI Marker-4.05, 1 μL Maurice cIEF pI Marker-9.99, 35 μL 1% Methyl Cellulose Solution, 2 μL Maurice cIEF 500 mM Arginine, 4 μL Ampholytes (Pharmalyte pH ranges 3-10), and water (added to make a final volume of 100 μL). On the Maurice analyzer (Protein Simple, Santa Clara, CA), Maurice cIEF Cartridges (PS-MC02-C) were used to generate imaging capillary isoelectric focusing spectra. The sample was focused for a total of 10 minutes. The analysis software installed on the instrument was used to analyze the absorbance of the 280 nm-focused protein.

In the activity detection experiment, 30 μL of CHO-S-hSIGLEC15 cells were added to the culture plate, and the antibody to be tested was added in a gradient dilution (90, 30, 10˜0.000152, 0.00051 μg/mL), and incubated at 4° C. for 30 min. The plate was washed with PBS, and diluted secondary antibody Anti-hIgG-Fc-Alex Flour 647 (RL1-H) (1:2000) was added, incubated at 4° C. for 30 min. The plate was washed once with PBS and the cells were resuspended for flow cytometry detection. MFI value was derived by FlowJo 7.6.1 analysis to calculate EC50.

Detailed results of 10C9, 10C9-S and 23F4 are shown in the table below. The results showed that these anti-SIGLEC15 antibodies have good stability and physical and chemical properties.

TABLE 5

| | | SEC | | cIEF | | | | |
|---|---|---|---|---|---|---|---|---|
| | | Purity | HIC | Acidic | Main | Alkali | | EC50 |
| Antibody | Treatment | (%) | (min) | peak (%) | peak (%) | peak (%) | PI | (μg/mL) |
| 10C9 | 0 d | — | 4.409 | 11.2 | 83.8 | 2.9 | 8.168 | 1.17 |
| | 4° C. 7 d | — | 4.375 | 10.5 | 84.2 | 2.8 | 8.177 | 0.85 |
| | 25° C. 7 d | — | 4.387 | 11.2 | 84.0 | 2.9 | 8.176 | 0.65 |
| | 40° C. 7 d | — | 4.375 | 25.9 | 67.2 | 4.0 | 8.151 | 0.60 |
| | freeze 1 | — | 4.361 | 11.2 | 84.0 | 2.8 | 8.173 | 0.62 |
| | freeze 10 | — | 4.368 | 10.8 | 84.1 | 2.7 | 8.168 | 0.81 |
| | pH 3.5 0 h | 98.17 | 4.649 | 18.9 | 71.8 | 2.9 | 8.746 | 0.44 |
| | pH 3.5 6 h | 96.02 | 4.628 | 14.8 | 78.9 | 3.8 | 8.656 | 0.11 |
| 10C9-S | 0 d | 96.00 | 3.139 | 8.5 | 68.4 | 3.5 | 7.724 | 1.79 |
| | 4° C. 7 d | 95.93 | 3.140 | 9.1 | 61.4 | 3.0 | 7.712 | 1.63 |
| | 25° C. 7 d | 95.77 | 3.143 | 9.4 | 59.9 | 3.1 | 7.718 | 1.64 |
| | 40° C. 7 d | 95.95 | 3.145 | 12.8 | 54.8 | 4.1 | 7.702 | 1.55 |
| | freeze 1 | 96.00 | 3.145 | 8.8 | 58.7 | 1.8 | 7.705 | 1.46 |
| | freeze 10 | 96.00 | 3.147 | 9.7 | 61.1 | 3.6 | 7.702 | 1.67 |
| | pH 3.5 0 h | 96.26 | 3.163 | 9.1 | 61.5 | 5.2 | 8.045 | 1.66 |
| | pH 3.5 6 h | 96.14 | 3.151 | 8.4 | 61.5 | 4.1 | 8.061 | 2.05 |
| 23F4 | 0 d | — | 2.477 | 13.0 | 81.1 | 2.4 | 8.264 | 0.1287 |
| | 4° C. 7 d | — | 2.473 | 12.5 | 80.6 | 2.6 | 8.261 | 0.1217 |
| | 25° C. 7 d | — | 2.488 | 13.7 | 80.0 | 2.9 | 8.286 | 0.1061 |
| | 40° C. 7 d | — | 2.507 | 19.1 | 77.1 | — | 8.258 | 0.1105 |
| | freeze 1 | — | 2.523 | 13.8 | 79.7 | 2.7 | 8.261 | 0.1252 |
| | freeze 10 | — | 2.536 | 13.5 | 80.0 | 2.5 | 8.269 | 0.1289 |
| | pH 3.5 0 h | 97.43 | 2.558 | 0.7 | 92.1 | 2.3 | 8.873 | 0.1484 |
| | pH 3.5 6 h | 97.38 | 2.566 | 3.1 | 88.2 | 3.7 | 8.891 | 0.2205 |

Note:
—: not detected.

### Example 6. Human PBMC Proliferation Assay

Proliferation assay was performed using human peripheral blood mononuclear cells (PBMCs). Human PBMCs (AllCells, Cat #: FPB003F-C) were stained with 5-(6)-carboxy-fluorescein succinimidyl ester (CFSE, Thermo Fisher, Cat #: C34554) according to the manufacturer's instructions. 0.01 μg/mL anti-CD3 monoclonal antibody (ACROBiosystems Inc., Cat #: CDE-M120a) was used to coat a 96 well plate. The coating was performed overnight at 4° C. The plate was washed three times with PBS. 5.00 μg/mL (final concentration) of human SIGLEC15 proteins (hSIGLEC15, ACROBiosystems Inc., Cat #: SG5-H5253) was added along with various concentrations of different anti-SIGLEC15 antibodies. The plate was incubated for 3-4 hours at 37° C. 2×10⁵ PBMC cells labeled with CFSE were added to each well and incubated for 72 hours at 37° C. with 5% CO₂. The PBMC cells were stained with anti-CD4 antibody (Biolegend, Cat #: 300514) and anti-CD8a antibody (Biolegend, Cat #: 301036) to conduct the flow cytometry analysis. Details of the incubation scheme and the result are shown in Table 6. The results showed that 23F4, 10C9 and 10C9-S reversed the inhibition of T cell proliferation caused by human SIGLEC15 protein, and the effect of reversing the inhibition of T cell proliferation was stronger with increasing dose.

TABLE 6

| | Protein and Dosage (μg/mL) | | | | | Proliferating | Proliferating |
|---|---|---|---|---|---|---|---|
| | Anti-CD3 | | | | | hCD4+ T | hCD8+ T |
| Group | antibody | hSIGLEC15 | 23F4 | 10C9-S | 5G12 | cell (%) | cell (%) |
| G1 | 0 | 0 | 0 | 0 | 0 | 3.43 | 4.66 |
| G2 | 0.01 | 0 | 0 | 0 | 0 | 84.30 | 80.75 |
| G3 | 0.01 | 5.00 | 0 | 0 | 0 | 32.65 | 25.60 |
| G4 | 0.01 | 5.00 | 3.00 | 0 | 0 | 34.95 | 26.20 |
| G5 | 0.01 | 5.00 | 10.00 | 0 | 0 | 65.15 | 53.60 |
| G6 | 0.01 | 5.00 | 30.00 | 0 | 0 | 66.30 | 57.75 |
| G7 | 0.01 | 5.00 | 0 | 3.00 | 0 | 40.70 | 34.35 |
| G8 | 0.01 | 5.00 | 0 | 10.00 | 0 | 60.05 | 49.90 |
| G9 | 0.01 | 5.00 | 0 | 30.00 | 0 | 68.60 | 61.05 |
| G10 | 0.01 | 5.00 | 0 | 0 | 10.00 | 43.80 | 33.75 |

### Example 7. Pharmacokinetics (PK) Analysis

A humanized SIGLEC15 mouse model (hSIGLEC15 mice) was engineered to express a chimeric SIGLEC15 protein (SEQ ID NO: 67) wherein a part of the extracellular region of the mouse SIGLEC15 protein was replaced with the corresponding human SIGLEC15 extracellular region. A detailed description regarding humanized SIGLEC15 mouse model can be found in PCT/CN2022/080427, which is incorporated herein by reference in its entirety.

The pharmacokinetic clearance rates of the anti-SIGLEC15 antibodies were determined in hSIGLEC15 mice. Specifically, the mice were placed into two groups (3 mice per group), and administered with 10 mg/kg of 23F4 or 5G12 by intravenous injection. Blood samples were collected 4 days before administration and 15 minutes, 4 hr, 1 day, 3 days, 7 days, 10 days, 14 days and 21 days after administration.

The serum levels of human antibodies were determined by sandwich ELISA. Briefly, Goat Anti-Human IgG (H+L) (Jackson ImmunoResearch Inc., Cat #: 109-005-088) was diluted to a final concentration of 2000 ng/mL, added to a 96-well plate (ELISA plate) at 100 μL/well, and then incubated overnight at 4° C. After the incubation, the plate was washed with PBS-T buffer (PBS supplemented with Tween™ 20) 4 times. Antibody-unbound areas were blocked with 2% BSA (bovine serum albumin) for 2 hours at 37° C. Afterwards, the plate was washed with PBS-T buffer 4 times. After washing, 100 μL of blocking buffer (2% BSA) was added to each well. The wells were sealed and incubated at 37° C. for 1 hour. After washing the plate using a plate washer, Peroxidase AffiniPure F(ab′)₂ Fragment Goat Anti-Human IgG, Fcγ fragment specific (Jackson ImmunoResearch Inc., Cat #: 109-036-098) was added at 100 μL/well to each well of the plate, and incubated at 37° C. for 1 hour. After washing the plate, tetramethylbenzidine (TMB) solution was added at 100 μL/well to the 96-well plate as the substrate. After incubating at room temperature in the dark, 100 μL stop solution (Beyotime, Cat #: P0215) was added to each well. A microplate reader was used to read the absorbance value of each well at wavelengths of 450 nm and 630 nm. Data analysis software GenS™ was used to analyze the data. The absorbance value and corresponding concentration of the calibration sample prepared by each test product was used to create a standard curve with four parameters (i.e., T_{1/2}, Cₘₐₓ, AUC_{0-21 day}, and CL). The standard curve was used to calculate the antibody concentration of each serum sample. A drug concentration-time curve was created using the calculated sample concentration at each time point. Phoenix™ WinNolin 8.3 was used to calculate the pharmacokinetic parameters. The results are shown in the table below.

TABLE 7

| Group | | T_{1/2} (day) | Cₘₐₓ (μg/mL) | AUC_{0-21 day} (day*μg/mL) | CL (mL/day/kg) |
|---|---|---|---|---|---|
| 5G12 | Mean | 4.19 | 66.39 | 277.41 | 34.43 |
| | SE | 0.25 | 3.71 | 1.97 | 0.50 |
| 10A7 | Mean | 11.51 | 117.56 | 635.58 | 11.35 |
| | SE | 0.49 | 5.41 | 13.96 | 0.22 |
| 23F4 | Mean | 9.79 | 184.10 | 969.47 | 7.97 |
| | SE | 0.65 | 5.91 | 16.04 | 0.28 |

T_{1/2}: Terminal Half Life;
Cₘₐₓ: Max Concentration;
AUC₀₋₂₁ _{day}: Area under Blood Concentration-time Curve 0-21 day
CL: Clearance

4 days before antibody administration, the antibody concentration was detected as 0 μg/mL (results not shown). As shown in FIGS. 2A-2C and Table 7, the results were consistent with typical pharmacokinetic characteristics, showing that after injection of anti-SIGLEC15 antibodies, the concentration of antibodies in the serum of hSIGLEC15 mice decreased over time. The half-life of 10A7, 23F4 and 5G12 in hSIGLEC15 mice were 11.51 days, 9.79 days and 4.19 days respectively.

In a similar experiment, the PK analysis results of 23F4-S and 10C9-S are shown in Table 8.

TABLE 8

| | | T_{1/2} | Cₘₐₓ | AUC_{0-35 day} | CL |
|---|---|---|---|---|---|
| Group | | (day) | (μg/mL) | (day*μg/mL) | (mL/day/kg) |
| 10C9-S | Mean | 4.62 | 64.44 | 235.10 | 42.31 |
| | CV | 9.2% | 3.0% | 3.8% | 3.7% |
| 23F4-S | Mean | 7.49 | 148.74 | 565.04 | 17.14 |
| | CV | 2.1% | 7.7% | 5.4% | 5.4% |

### Example 8. Anti-Tumor Activity in hSIGLEC15 Mice

Anti-Tumor Activity of 23F4 in Combination with Anti-PD-L1 Antibody

The hSIGLEC15 mice were used to determine the anti-tumor activity of anti-SIGLEC15 antibodies. About 5×10⁵ MC38 cells were injected subcutaneously in hSIGLEC15 mice, and when the tumor volume grew to about 100-150 mm³, the mice were divided to different groups based on tumor size (5 mice per group). The treatment groups were randomly selected for 23F4 treatment, Atezolizumab analog treatment or a combination of Atezolizumab analog and 23F4 treatment. The control group mice were injected with phosphate buffer saline (PBS). The frequency of administration was twice a week (6 administrations in total). The tumor volume was measured twice a week and the body weight of the mice was weighed as well. Euthanasia was performed when the tumor volume of the mouse reached 3000 mm³. Details of the administration scheme are shown in the table below.

TABLE 9

| | No. of | | Dosage | | | Total No. of |
|---|---|---|---|---|---|---|
| Group | mice | Antibodies | (mg/kg) | Route | Frequency | administration |
| G1 | 5 | PBS | — | i.p. | BIW | 6 |
| G2 | 5 | Atezolizumab analog | 3 mg/kg | i.p. | BIW | 6 |
| G3 | 5 | 23F4 | 1 mg/kg | i.p. | BIW | 6 |
| G4 | 5 | 23F4 | 3 mg/kg | i.p. | BIW | 6 |
| G5 | 5 | 23F4 | 10 mg/kg | i.p. | BIW | 6 |
| G6 | 5 | Atezolizumab analog | 3 mg/kg | i.p. | BIW | 6 |
| | | 23F4 | 10 mg/kg | i.p. | BIW | 6 |

The length of the long axis and the short axis of the tumor were measured and the volume of the tumor was calculated as 0.5×(long axis)×(short axis)². The weight of the mice was also measured twice a week.

The tumor growth inhibition percentage (TGI %) was calculated using the following formula: TGI (%)=[1−(Ti−T0)/(Vi−V0)]×100. Ti is the average tumor volume in the treatment group on day i. T0 is the average tumor volume in the treatment group on day zero. Vi is the average tumor volume in the control group on day i. V0 is the average tumor volume in the control group on day zero.

T-test was performed for statistical analysis. P<0.05 is a threshold to indicate significant difference.

The weight of mice in different groups all increased. On the day of group assignment (Day 0), the average weight of each group was in the range of 19.3 g-19.7 g. At the end of the experiment (Day 25), the average weight of each group was in the range of 22.8 g-24.9 g. The average weight of each group was in the range of 118.0%-126.7%. The results showed that the tested antibodies were well tolerated and were not obviously toxic to the mice.

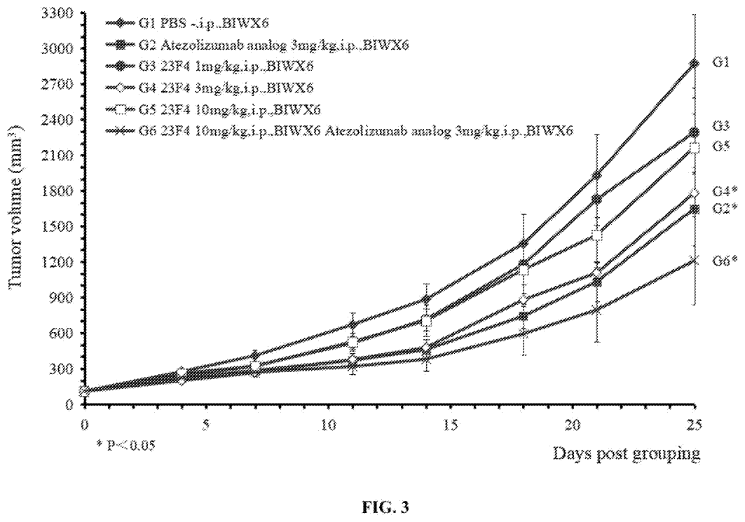

The tumor size data in groups treated with the antibodies are shown in FIG. 3. Table 10 below summarizes the results for this experiment, including the tumor volumes on the day of grouping (day 0), 14 days after grouping (day 14) and at the end of the experiment (day 25); the survival rate of the mice; Tumor Growth Inhibition value (TGI %); and the statistical differences (P value) of tumor volume between the treatment and control groups.

TABLE 10

| | Tumor volume (mm³) | | | | | P |
|---|---|---|---|---|---|---|
| Group | Day 0 | Day 14 | Day 25 | Survival | TGI % | value |
| G1 | 108 ± 4 | 884 ± 132 | 2872 ± 420 | 5/5 | NA | NA |
| G2 | 108 ± 5 | 459 ± 84 | 1647 ± 313 | 5/5 | 44.3 | 0.047 |
| G3 | 108 ± 4 | 714 ± 169 | 2291 ± 433 | 5/5 | 21.0 | 0.364 |
| G4 | 108 ± 5 | 477 ± 25 | 1780 ± 167 | 5/5 | 39.5 | 0.042 |
| G5 | 108 ± 5 | 705 ± 132 | 2163 ± 506 | 5/5 | 25.7 | 0.312 |
| G6 | 108 ± 5 | 379 ± 96 | 1208 ± 371 | 5/5 | 60.2 | 0.018 |

As shown in FIG. 3 and Table 10, compared with the control group (G1), the tumor growth in the treatment groups (G2 and G6) were inhibited to different extents. In treatment groups G3-G5, which were treated with 23F4 only, G4 showed the best tumor suppressive effect. In addition, 23F4 in combination with Atezolizumab analog (G6) obtained better tumor inhibitory effect as compared to 23F4 treatment group (G5) or anti-PD-L1 antibody treatment group (G2), indicating that anti-SIGLEC15 antibody enhanced the anti-tumor efficacy of anti-PD-L1 antibody in MC38 model.

Anti-Tumor Activity of 23F4 at a Dosage of 3 mg/Kg

In another experiment, about 5×10⁵ MC38 cells were injected subcutaneously in hSIGLEC15 mice to determine the anti-tumor activity of 23F4, and when the tumor volume grew to about 100 mm³, the mice were divided to a control group and two treatment groups based on tumor size (7 mice per group). The treatment groups were randomly selected for 23F4 treatment (G2, 3 mg/kg) or 5G12 treatment (G3, 10 mg/kg). The control group mice were injected with PBS (G1). The frequency of administration was twice a week (6 times of administrations in total). The tumor volume was measured twice a week and the body weight of the mice was weighed as well.

The weight of mice in different groups all increased. On Day 0, the average weight of each group was in the range of 19.9 g-20.1 g. At the end of the experiment (Day 21), the average weight of each group was in the range of 23.1 g-23.6 g. The average weight of each group was in the range of 115.6%-118.1%. The results showed that the tested antibodies were well tolerated and were not obviously toxic to the mice.

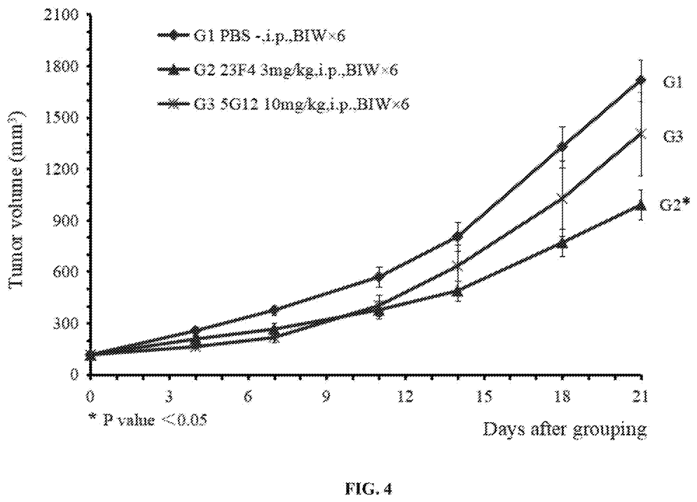

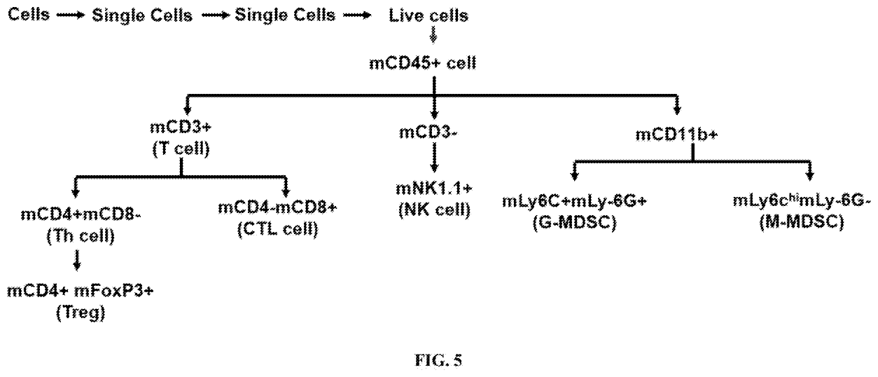

The tumor size in groups treated with the antibodies are shown in FIG. 4 and Table 11, which show that compared with the control group (G1), the tumor growth in the treatment groups (G2 and G3) were inhibited to different extents, and 23F4 at a dose of 3 mg/kg obtained better tumor inhibitory effect as compared to 5G12.

TABLE 11

| | Tumor volume (mm³) | | | | | P |
|---|---|---|---|---|---|---|
| Group | Day 0 | Day 11 | Day 21 | Survival | TGI % | value |
| G1 | 114 ± 2 | 568 ± 58 | 1717 ± 120 | 7/7 | NA | NA |
| G2 | 114 ± 2 | 376 ± 50 | 990 ± 88 | 7/7 | 45.4 | 0 |
| G3 | 114 ± 3 | 401 ± 61 | 1405 ± 244 | 7/7 | 19.5 | 0.273 |

### TILs Analysis in MC38 Tumor Model

In another experiment, about 5×10⁵ MC38 cells were injected subcutaneously in hSIGLEC15 mice to determine the anti-tumor activity of 23F4 and 23F4-S, and when the tumor volume grew to about 100 mm³, the mice were divided to a control group and four treatment groups based on tumor size (7 mice per group). The treatment groups were randomly selected for 23F4 treatment (G2 (1 mg/kg) and G3 (3 mg/kg)) or 23F4-S treatment (G4 (1 mg/kg)). The control group mice were injected with PBS (G1). The frequency of administration was twice a week (6 times of administrations in total). The tumor volume was measured twice a week and the body weight of the mice was weighed as well.

The weight of mice in different groups all increased. On Day 0, the average weight of each group was in the range of 20.8 g-21.3 g. At the end of the experiment (Day 20), the average weight of each group was in the range of 22.9 g-23.9 g. The average weight of each group was in the range of 109.7%-114.0%. The results showed that the tested antibodies were well tolerated and were not obviously toxic to the mice.

The tumor size data in groups treated with the antibodies are shown in Table 12, which show that compared with the control group (G1), the tumor growth in the treatment groups (G2-G4) were inhibited to different extents.

TABLE 12

| | Tumor volume (mm³) | | | | | P |
|---|---|---|---|---|---|---|
| Group | Day 0 | Day 9 | Day 20 | Survival | TGI % | value |
| G1 | 115 ± 3 | 553 ± 67 | 1574 ± 161 | 7/7 | NA | NA |
| G2 | 115 ± 3 | 474 ± 60 | 1271 ± 220 | 7/7 | 20.8 | 0.288 |
| G3 | 115 ± 4 | 353 ± 42 | 1023 ± 109 | 7/7 | 37.8 | 0.015 |
| G4 | 115 ± 3 | 477 ± 30 | 1214 ± 144 | 7/7 | 24.6 | 0.122 |

At the end of the experiment, the tumor tissue was collected, digested, and then re-suspended as a single cell suspension for flow cytometry analysis. Anti-mouse CD45 antibody APC/Cyanine7 anti-mouse CD45 antibody (BioLegend, Cat #: 103116), anti-mouse CD16/32 antibody Purified anti-mouse CD16/32 antibody (BioLegend, Cat #: 101302), anti-mouse CD3 antibody PerCP/Cyanine5.5 anti-mouse CD3& antibody (BioLegend, Cat #: 100328), anti-mouse CD4 antibody FITC anti-mouse CD4 antibody (BioLegend, Cat #: 100406), anti-mouse CD8a antibody Brilliant Violet 605™ anti-mouse CD8a antibody (BioLegend, Cat #: 100744), anti-mouse NK1.1 antibody Brilliant Violet 421™ anti-mouse NK1.1 antibody (BioLegend, Cat #: 108732), anti-mouse CD11b antibody PE anti-mouse/human CD11b antibody (BioLegend, Cat #: 101208), anti-mouse ly-6G antibody Alexa Fluor®700 anti-mouse ly-6G antibody (BioLegend, Cat #: 127622), anti-mouse mLy-6C antibody APC anti-mouse mLy-6C antibody (BioLegend. Cat #: 128016), anti-mouse Foxp3 antibody (eBioscience, Cat #: 25-5773-82) were used for cell staining before flow cytometry. The cell sorting strategy for TILs (tumor-infiltrating lymphocytes) analysis is shown in FIG. 5.

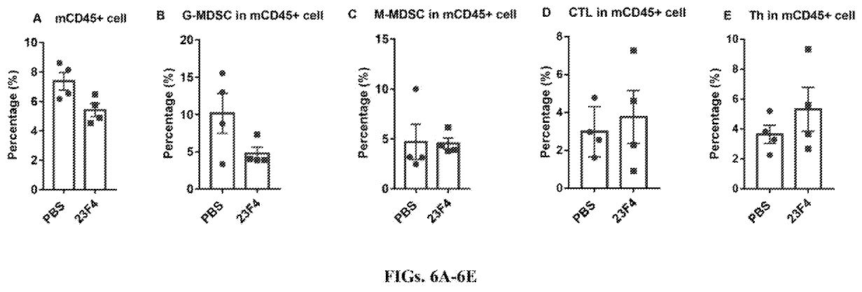

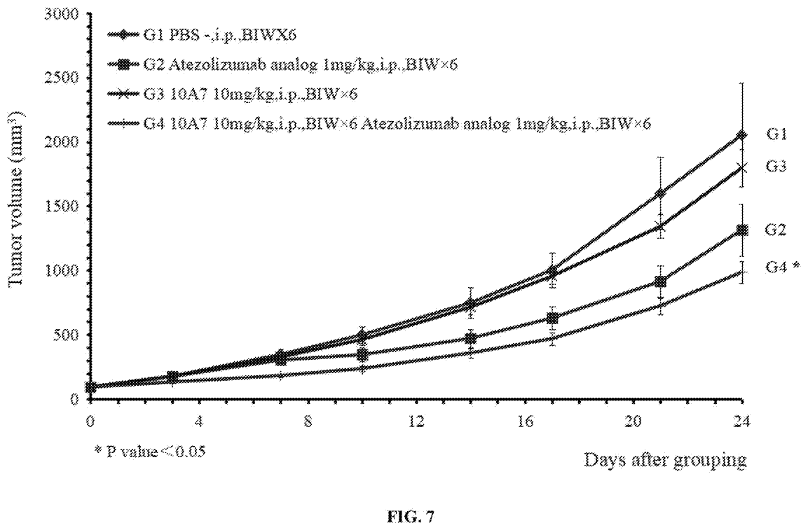

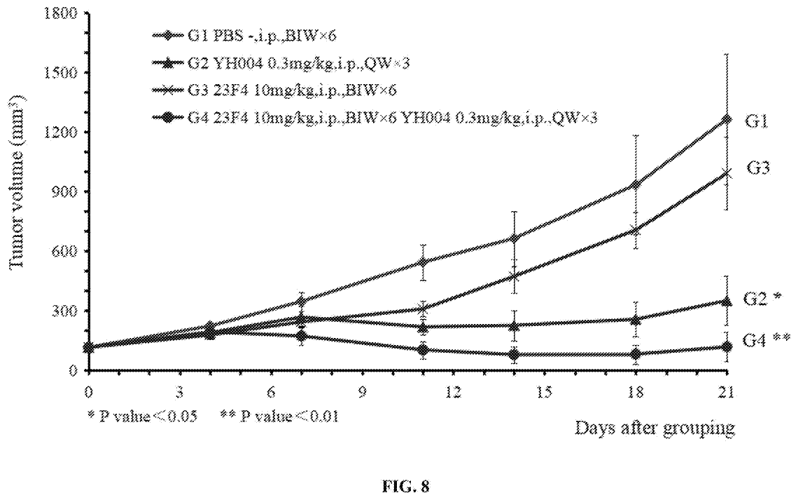

As shown in FIGS. 6A-6E, anti-SIGLEC15 antibodies can significantly reduce the number of G-MDSC (granulocytic myeloid derived suppressor cells) in the tumor microenvironment, and then relieve the immunosuppression caused by G-MDSC, which is manifested as a slight increase in the number of T helper (Th) cells and CTL (cytotoxic T lymphocytes). Compared with M-MDSC (monocytic myeloid-derived suppressor cells), G-MDSC occupies a higher proportion in the tumor microenvironment, so the effect of G-MDSC on the immunosuppressive effect may be greater than that of M-MDSC.

### Anti-Tumor Activity of 10C9

A total of fifteen hSIGLEC15 mice were subcutaneously injected with MC38 cells (5×10⁵/mouse). When the tumor reached a volume of 100 mm³, the mice were randomly placed into 3 groups, with 5 mice in each group. The treatment groups were treated with 10C9 or the positive control antibody 5G12 respectively by intraperitoneal injection at a dose of 3 mg/kg, and the control group was injected with PBS, and administered twice a week. The body weight and tumor volume of the mice were measured twice a week until the end of the experiment after 3 weeks.

During the entire treatment period, the average body weight of the mice in the control group and the treatment groups increased steadily and there was no significant difference between the groups, indicating that these 10C9 was not obviously toxic to the mice. Table 13 below showed the TGI % (21 days after grouping) results for each group, compared with the control group, the tumor growth in the treatment groups were inhibited to different extents, wherein the 10C9 treatment group obtained better tumor inhibitory effect as compared to 5G12.

TABLE 13

| | | Average tumor | | |
|---|---|---|---|---|
| Group | Antibody | volume (mm³) | TGI% | P value |
| G1 | PBS | 1632 ± 238 | NA | NA |
| G2 | 5G12 (3 mg/kg) | 1293 ± 163 | 21.9% | 0.274 |
| G3 | 10C9 (3 mg/kg) | 1157 ± 225 | 30.8% | 0.185 |

Anti-Tumor Activity of 10A7 in Combination with Anti-PD-L1 Antibody

A total of twenty hSIGLEC15 mice were subcutaneously injected with MC38 cells (5×10⁵/mouse). When the tumor reached a volume of 100 mm³, the mice were divided to a control group and 3 treatment groups based on tumor size (5 mice per group). The treatment groups were randomly selected for an anti-PD-L1 antibody Atezolizumab analog treatment, 10A7 treatment or a combination of Atezolizumab analog and 10A7 treatment. The control group mice were injected with PBS. The tumor volume was measured twice a week and the body weight of the mice was weighed as well. Euthanasia was performed when the tumor volume of the mouse reached 3000 mm³. Details of the administration scheme are shown in the table below.

TABLE 14

| | No. of | | Dosage | | | Total No. of |
|---|---|---|---|---|---|---|
| Group | mice | Antibodies | (mg/kg) | Route | Frequency | administration |
| G1 | 5 | PBS | — | i.p. | BIW | 6 |
| G2 | 5 | Atezolizumab analog | 1 mg/kg | i.p. | BIW | 6 |
| G3 | 5 | 10A7 | 10 mg/kg | i.p. | BIW | 6 |
| G4 | 5 | Atezolizumab analog | 1 mg/kg | i.p. | BIW | 6 |
| | | 10A7 | 10 mg/kg | | | 6 |

Atezolizumab is a humanized anti-PD-L1 monoclonal antibody. The heavy chain sequence and the light chain sequence are shown in SEQ ID NOs: 65-66.

During the entire treatment period, the average body weight of the mice in the control group and the treatment groups increased steadily and there was no significant difference between the groups, indicating that these antibodies were not obviously toxic to the animals.

The tumor size in groups treated with the antibodies are shown in FIG. 7. The Table 15 below showed the TGI % (24 days after grouping) results for each group, compared with the control group, the tumor growth in the treatment groups were inhibited to different extents, wherein the anti-SIGLEC15 antibody 10A7 in combination with anti-PD-L1 antibody treatment group obtained better tumor inhibitory effect as compared to 10A7 treatment group or anti-PD-L1 antibody treatment group.

TABLE 15

| | Tumor volume (mm³) | | | | |
|---|---|---|---|---|---|
| Group | Day 0 | Day 10 | Day 24 | TGI% | P value |
| G1 | 93 ± 3 | 498 ± 65 | 2052 ± 403 | NA | NA |
| G2 | 93 ± 5 | 345 ± 56 | 1314 ± 202 | 37.7 | 0.140 |
| G3 | 93 ± 3 | 462 ± 40 | 1798 ± 143 | 13.0 | 0.569 |
| G4 | 93 ± 3 | 236 ± 19 | 986 ± 82 | 54.4 | 0.032 |

### Example 9. Anti-Tumor Activity in C57BL/6 Mice

The C57BL/6 mice were subcutaneously injected with MC38 cells (5×10⁵/mouse). When the tumor reached a volume of 100 mm³, the mice were randomly divided into 3 groups, with 7 mice in each group. The treatment groups were treated with 10C9-S by intraperitoneal injection, and the control group was injected with PBS, and administered twice a week. The body weight and tumor volume of the mice were measured twice a week until the end of the experiment after 24 days. Details of the administration scheme are shown in the table below.

TABLE 16

| | No. of | | Dosage | | | Total No. of |
|---|---|---|---|---|---|---|
| Group | mice | Antibodies | (mg/kg) | Route | Frequency | administration |
| G1 | 7 | PBS | — | i.p. | BIW | 6 |
| G2 | 7 | 10C9-S | 1 mg/kg | i.p. | BIW | 6 |
| G3 | 7 | 10C9-S | 10 mg/kg | i.p. | BIW | 6 |

During the entire treatment period, the average body weight of the mice in the control group and the treatment groups increased steadily and there was no significant difference between the groups, indicating that 10C9-S were not obviously toxic to the animals. Table 17 below showed the TGI % (24 days after grouping) results for each group, compared with the control group, the tumor growth in 10C9-S treatment groups were inhibited to different extents.

TABLE 17

| | Average tumor volume (mm³) | | | | |
|---|---|---|---|---|---|
| Group | Day 0 | Day 10 | Day 24 | TGI% | P value |
| G1 | 119 ± 3 | 486 ± 69 | 1803 ± 268 | NA | NA |
| G2 | 119 ± 5 | 337 ± 40 | 1322 ± 305 | 28.6 | 0.259 |
| G3 | 119 ± 5 | 340 ± 50 | 1335 ± 317 | 27.8 | 0.282 |

### Example 10. Anti-Tumor Activity in h4-1BB Mice

A humanized 4-1BB mouse model (h4-1BB mice) was engineered to express a chimeric 4-1BB protein (SEQ ID NO: 68) wherein a part of the extracellular region of the mouse 4-1BB protein was replaced with the corresponding human 4-1BB extracellular region. A detailed description regarding humanized 4-1BB mouse model can be found in PCT/CN2021/087867, which is incorporated herein by reference in its entirety.

About 5×10⁵ mouse colon cancer cell MC38 were injected subcutaneously in h4-1BB mice, and when the tumor volume grew to about 100 mm³, the mice were divided to a control group and 3 treatment groups based on tumor size (5 mice per group). The treatment groups were randomly selected for an anti-4-1BB antibody YH004 treatment (G2), 23F4 treatment (G3) or a combination of YH004 and 23F4 treatment (G4). A detailed description regarding YH004 can be found in PCT/CN2019/105315, which is incorporated herein by reference in its entirety. The control group mice were injected with phosphate buffer saline (PBS) (G1). The tumor volume was measured twice a week and the body weight of the mice was weighed as well. Euthanasia was performed when the tumor volume of the mouse reached 3000 mm³. Details of the administration scheme are shown in the table below.

TABLE 18

| | No. of | | Dosage | | | Total No. of |
|---|---|---|---|---|---|---|
| Group | mice | Antibodies | (mg/kg) | Route | Frequency | administration |
| G1 | 5 | PBS | — | i.p. | BIW | 6 |
| G2 | 5 | YH004 | 0.3 mg/kg | i.p. | QW | 3 |
| G3 | 5 | 23F4 | 10 mg/kg | i.p. | BIW | 6 |
| G4 | 5 | YH004 | 0.3 mg/kg | i.p. | QW | 3 |
| | | 23F4 | 10 mg/kg | | BIW | 6 |

The weight of mice in different groups all increased. On the day of group assignment (Day 0), the average weight of each group was in the range of 19.2 g-19.4 g. At the end of the experiment (Day 21), the average weight of each group was in the range of 20.9 g-22.0 g. The average weight of each group was in the range of 109.0%-114.4%. The results showed that the tested antibodies were well tolerated and were not obviously toxic to the mice.

As shown in FIG. 8 and Table 19, compared with the control group, the tumor growth in the treatment groups were inhibited to different extents. Among the treatment groups, the anti-SIGLEC15 antibody in combination with anti-4-1BB antibody could significantly inhibit tumor growth with superior efficacy.

TABLE 19

| | | | | | | P value | |
|---|---|---|---|---|---|---|---|
| | Tumor volume (mm³) | | | | | Body | Tumor |
| Group | Day 0 | Day 11 | Day 21 | Survival | TGI % | weight | Volume |
| G1 | 115 ± 5 | 543 ± 89 | 1264 ± 327 | 5/5 | NA | NA | NA |
| G2 | 115 ± 7 | 218 ± 40 | 350 ± 125 | 5/5 | 79.5 | 0.290 | 0.031 |
| G3 | 115 ± 7 | 308 ± 40 | 991 ± 183 | 5/5 | 23.8 | 0.688 | 0.487 |
| G4 | 115 ± 7 | 102 ± 43 | 117 ± 73 | 5/5 | 99.8 | 0.193 | 0.009 |

### Example 11. Anti-Tumor Activity in hOX40 Mice

A humanized OX40 mouse model (hOX40 mice) was engineered to express a chimeric OX40 protein (SEQ ID NO: 69) wherein a part of the extracellular region of the mouse OX40 protein was replaced with the corresponding human OX40 extracellular region. A detailed description regarding humanized OX40 mouse model can be found in PCT/CN2017/099575, which is incorporated herein by reference in its entirety.

About 5×10⁵ mouse colon cancer cell MC38 were injected subcutaneously in hOX40 mice, and when the tumor volume grew to about 100 mm³, the mice were divided to a control group and 4 treatment groups based on tumor size (5 mice per group). The treatment groups were randomly selected for an anti-OX40 antibody YH002 treatment (G2), 10A7 treatment (G3), a combination of YH002 and 10A7 treatment (G4) or a combination of YH002 and 10C9 treatment (G5). A detailed description regarding YH002 can be found in PCT/CN2017/112832, which is incorporated herein by reference in its entirety. The control group mice were injected with an equal volume of PBS (G1). Details of the administration scheme are shown in the table below.

TABLE 20

| | No. of | | Dosage | | | Total No. of |
|---|---|---|---|---|---|---|
| Group | mice | Antibodies | (mg/kg) | Route | Frequency | administration |
| G1 | 5 | PBS | — | i.p. | BIW | 6 |
| G2 | 5 | YH002 | 1 mg/kg | i.p. | QW | 3 |
| G3 | 5 | 10A7 | 10 mg/kg | i.p. | BIW | 6 |
| G4 | 5 | YH002 | 1 mg/kg | i.p. | QW | 3 |
| | | 10A7 | 10 mg/kg | | BIW | 6 |
| G5 | 5 | YH002 | 1 mg/kg | i.p. | QW | 3 |
| | | 10C9 | 10 mg/kg | | BIW | 6 |

The weight of mice in different groups all increased. On the day of group assignment (Day 0), the average weight of each group was in the range of 19.7 g-20.1 g. At the end of the experiment (Day 24), the average weight of each group was in the range of 22.2 g-25.8 g. The average weight of each group was in the range of 111.8%-129.3%. The results showed that the tested antibodies were well tolerated and were not obviously toxic to the mice.

Table 21 below summarizes the results for this experiment, including the tumor volumes on the day of grouping (day 0), 14 days after the grouping (day 14) and at the end of the experiment (day 24); TGI and the P value of tumor volume between the treatment and control groups. The results showed that anti-OX40 antibody in combination with anti-SIGLEC15 antibodies 10A7 or 10C9 could significantly inhibit tumor growth with superior efficacy.

TABLE 21

| | Tumor volume (mm³) | | | | |
|---|---|---|---|---|---|
| Group | Day 0 | Day 14 | Day 24 | TGI% | P value |
| G1 | 109 ± 2 | 978 ± 89 | 2976 ± 273 | NA | NA |
| G2 | 109 ± 4 | 373 ± 114 | 915 ± 249 | 71.9 | 0.001 |
| G3 | 109 ± 4 | 935 ± 99 | 2968 ± 275 | 0.3 | 0.984 |
| G4 | 109 ± 5 | 143 ± 47 | 218 ± 125 | 96.2 | 1.58E−05 |
| G5 | 109 ± 5 | 217 ± 50 | 419 ± 134 | 89.2 | 3.02E−05 |

### Example 12. In Vivo Toxicity of Anti-SIGLEC15 Antibodies

In vivo toxicity of the anti-SIGLEC15 antibodies in hSIGLEC15 mice was evaluated. The hSIGLEC15 mice were placed into different groups (3 mice per group) by weight, the treatment groups were treated with 10A7, 23F4 or the positive control antibody 5G12 respectively by intraperitoneal injection at a dose of 30 mg/kg, and the control group was injected with PBS. The treatment was administered on day 0, day 3, day 7 and day 10. The body weight of the mice were measured twice a week until the end of the experiment. On day 11, day 16 and day 21, the peripheral blood and serum from the hSIGLEC15 mice were collected and analyzed using the biochemical test (alanine transaminase (ALT) and aspartate (AST)) and hematology test (complete blood count, including white blood cell count (WBC), red blood cell count (RBC), hemoglobin (HGB), hematocrit or packed cell volume (HCT), mean corpuscle volume (MCV), mean corpuscular hemoglobin (MCH), mean corpuscular hemoglobin concentration (MCHC), platelet count (PLT), lymphocyte percentage (LYMPH %), monocyte percentage (MONO %), neutrophil percentage (NEUT %)).

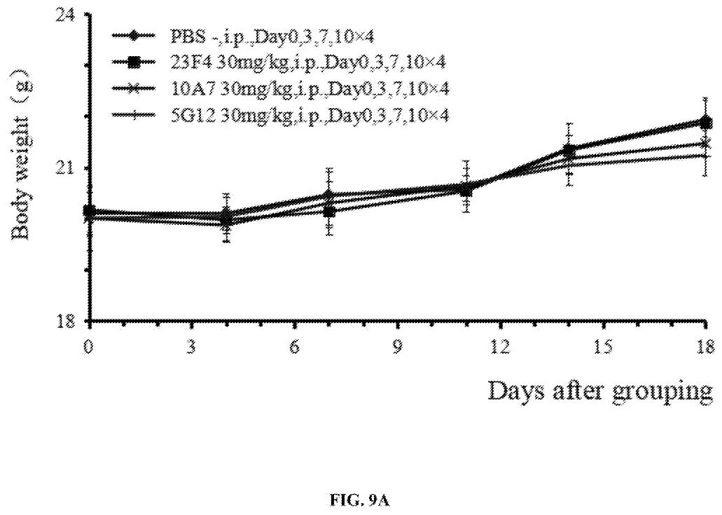

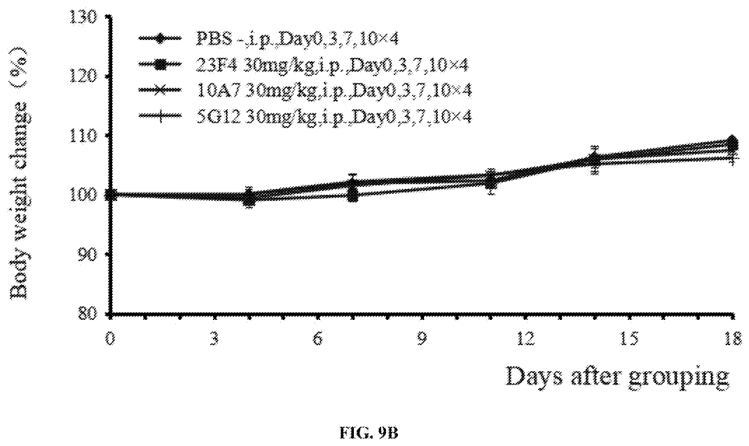

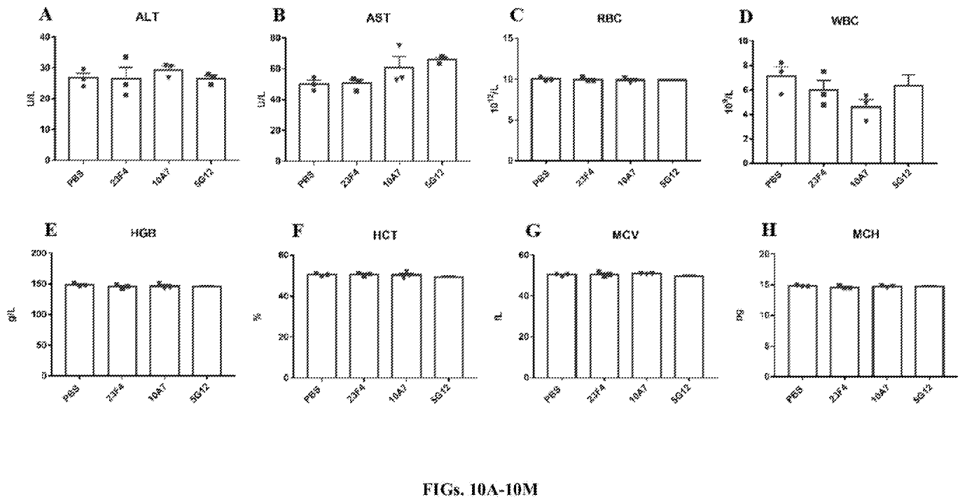

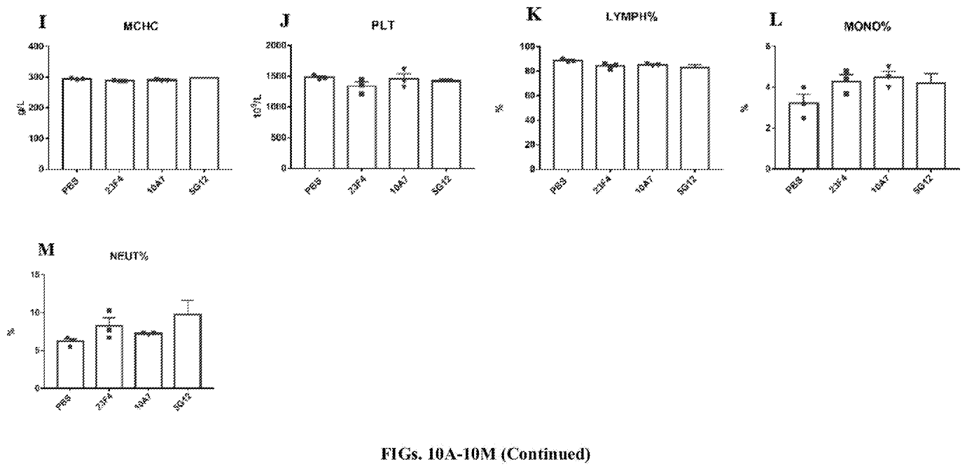

As shown in FIGS. 9A-9B, the average body weight of the mice in the control group and the treatment groups increased steadily during the entire treatment period. FIGS. 10A-10M show the hematology and biochemical test results, there was no significant difference between the groups. These results indicating that anti-SIGLEC15 antibodies 23F4 and 10A7 were not toxic to the mice.

Similar to the in vivo toxicity validation of 23F4, the toxicity analysis of 10C9 and 10C9-S were also performed. The results show that the average body weight of the mice in the control group and the treatment groups increased steadily during the entire treatment period, the hematology and biochemical test results was no significant difference between the groups. These results indicate that anti-SIGLEC15 antibodies were not obviously toxic to the mice.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An antibody or antigen-binding fragment thereof that binds to SIGLEC15 (Sialic acid binding ig-like lectin 15) comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 1, 2, 3, respectively, and the selected VL CDRs 1, 2, 3 amino acid sequences are set forth in SEQ ID NOs: 4, 5, 6, respectively;
(2) the selected VH CDRs 1, 2, 3 amino acid sequences are set forth in SEQ ID NOs: 7, 8, 9, respectively, and the selected VL CDRs 1, 2, 3 amino acid sequences are set forth in SEQ ID NOs: 10, 11, 12, respectively;
(3) the selected VH CDRs 1, 2, 3 amino acid sequences are set forth in SEQ ID NOs: 13, 14, 15, respectively, and the selected VL CDRs 1, 2, 3 amino acid sequences are set forth in SEQ ID NOs: 16, 17, 18, respectively;
(4) the selected VH CDRs 1, 2, 3 amino acid sequences are set forth in SEQ ID NOs: 19, 20, 21, respectively, and the selected VL CDRs 1, 2, 3 amino acid sequences are set forth in SEQ ID NOs: 22, 23, 24, respectively;
(5) the selected VH CDRs 1, 2, 3 amino acid sequences are set forth in SEQ ID NOs: 25, 26, 27, respectively, and the selected VL CDRs 1, 2, 3 amino acid sequences are set forth in SEQ ID NOs: 28, 29, 30, respectively; and
(6) the selected VH CDRs 1, 2, 3 amino acid sequences are set forth in SEQ ID NOs: 31, 32, 33, respectively, and the selected VL CDRs 1, 2, 3 amino acid sequences are set forth in SEQ ID NOs: 34, 35, 36, respectively.

2. The antibody or antigen-binding fragment thereof of claim 1, wherein the VH comprises CDRs 1, 2, 3 with the amino acid sequences set forth in SEQ ID NOs: 1, 2, and 3 respectively, and the VL comprises CDRs 1, 2, 3 with the amino acid sequences set forth in SEQ ID NOs: 4, 5, and 6, respectively, according to Kabat definition.

3. The antibody or antigen-binding fragment thereof of claim 1, wherein the VH comprises CDRs 1, 2, 3 with the amino acid sequences set forth in SEQ ID NOs: 7, 8, and 9, respectively, and the VL comprises CDRs 1, 2, 3 with the amino acid sequences set forth in SEQ ID NOs: 10, 11, and 12, respectively, according to Kabat definition.

4. The antibody or antigen-binding fragment thereof of claim 1, wherein the VH comprises CDRs 1, 2, 3 with the amino acid sequences set forth in SEQ ID NOs: 13, 14, and 15, respectively, and the VL comprises CDRs 1, 2, 3 with the amino acid sequences set forth in SEQ ID NOs: 16, 17, and 18, respectively, according to Kabat definition.

5. The antibody or antigen-binding fragment thereof of claim 1, wherein the VH comprises CDRs 1, 2, 3 with the amino acid sequences set forth in SEQ ID NOs: 19, 20, and 21, respectively, and the VL comprises CDRs 1, 2, 3 with the amino acid sequences set forth in SEQ ID NOs: 22, 23, and 24, respectively, according to Chothia definition.

6. The antibody or antigen-binding fragment thereof of claim 1, wherein the VH comprises CDRs 1, 2, 3 with the amino acid sequences set forth in SEQ ID NOs: 25, 26 and 27, respectively, and the VL comprises CDRs 1, 2, 3 with the amino acid sequences set forth in SEQ ID NOs: 28, 29, and 30, respectively, according to Chothia definition.

7. The antibody or antigen-binding fragment thereof of claim 1, wherein the VH comprises CDRs 1, 2, 3 with the amino acid sequences set forth in SEQ ID NOs: 31, 32, and 33, respectively, and the VL comprises CDRs 1, 2, 3 with the amino acid sequences set forth in SEQ ID NOs: 34, 35, and 36, respectively, according to Chothia definition.

8. The antibody or antigen-binding fragment thereof of any one of claims 1-7, wherein the antibody or antigen-binding fragment thereof specifically binds to human, mouse, monkey, or dog SIGLEC15.

9. The antibody or antigen-binding fragment thereof of any one of claims 1-8, wherein the antibody or antigen-binding fragment thereof is a human antibody or antigen-binding fragment thereof, a single-chain variable fragment (scFv), a one-armed antibody, and or a multi-specific antibody (e.g., a bispecific antibody).

10. The antibody or antigen-binding fragment thereof of any one of claims 1-9, wherein the antibody or antigen-binding fragment thereof is a human IgG1 antibody or antigen-binding fragment thereof or a human IgG4 antibody or antigen-binding fragment thereof.

11. A nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 1, 2, and 3, respectively, and wherein the VH, when paired with a light chain variable region (VL) comprising the amino acid sequence set forth in SEQ ID NO: 38 binds to SIGLEC15;
(2) an immunoglobulin light chain or a fragment thereof comprising a VL comprising complementarity determining regions (CDRs) 1, 2, and 3 comprising the amino acid sequences set forth in SEQ ID NOs: 4, 5, and 6, respectively, and wherein the VL, when paired with a VH comprising the amino acid sequence set forth in SEQ ID NO: 37 binds to SIGLEC15;
(3) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 7, 8, and 9, respectively, and wherein the VH, when paired with a light chain variable region (VL) comprising the amino acid sequence set forth in SEQ ID NO: 40 binds to SIGLEC15;
(4) an immunoglobulin light chain or a fragment thereof comprising a VL comprising complementarity determining regions (CDRs) 1, 2, and 3 comprising the amino acid sequences set forth in SEQ ID NOs: 10, 11, and 12, respectively, and wherein the VL, when paired with a VH comprising the amino acid sequence set forth in SEQ ID NO: 39 binds to SIGLEC15;
(5) an immunoglobulin heavy chain or a fragment thereof comprising a VH comprising complementarity determining regions (CDRs) 1, 2, and 3 comprising the amino acid sequences set forth in SEQ ID NOs: 13, 14, and 15, respectively, and wherein the VH, when paired with a light chain variable region (VL) comprising the amino acid sequence set forth in SEQ ID NO: 42 binds to SIGLEC15; or
(6) an immunoglobulin light chain or a fragment thereof comprising a VL comprising CDRs 1, 2, and 3 comprising the amino acid sequences set forth in SEQ ID NOs: 16, 17 and 18, respectively, and wherein the VL, when paired with a VH comprising the amino acid sequence set forth in SEQ ID NO: 41 binds to SIGLEC15.

12. The nucleic acid of claim 11, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 1, 2, and 3, respectively.

13. The nucleic acid of claim 11, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 4, 5, and 6, respectively.

14. The nucleic acid of claim 11, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 7, 8, and 9, respectively.

15. The nucleic acid of claim 11, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 10, 11, and 12, respectively.

16. The nucleic acid of claim 11, wherein the nucleic acid comprises a polynucleotide encoding a polypeptide comprising an immunoglobulin heavy chain or a fragment thereof comprising a VH comprising CDRs 1, 2, and 3 comprising the amino acid sequences set forth in SEQ ID NOs: 13, 14, and 15, respectively.

17. The nucleic acid of claim 11, wherein the nucleic acid comprises a polynucleotide encoding a polypeptide comprising an immunoglobulin light chain or a fragment thereof comprising a VL comprising CDRs 1, 2, and 3 comprising the amino acid sequences set forth in SEQ ID NOs: 16, 17, and 18, respectively.

18. The nucleic acid of any one of claims 11-17, wherein the VH when paired with a VL specifically binds to human, mouse, monkey, or dog SIGLEC15, or the VL when paired with a VH specifically binds to human, mouse, monkey, or dog SIGLEC15.

19. The nucleic acid of any one of claims 11-18, wherein the immunoglobulin heavy chain or the fragment thereof is a human immunoglobulin heavy chain or a fragment thereof (e.g., a human IgG1 heavy chain or a fragment thereof; or a human IgG4 heavy chain or a fragment thereof), and the immunoglobulin light chain or the fragment thereof is a human immunoglobulin light chain or a fragment thereof.

20. The nucleic acid of any one of claims 11-19, wherein the nucleic acid encodes a single-chain variable fragment (scFv), a one-armed antibody, a multi-specific antibody (e.g., a bispecific antibody), or a chimeric antigen receptor (CAR).

21. The nucleic acid of any one of claims 11-20, wherein the nucleic acid is cDNA.

22. A vector comprising one or more of the nucleic acids of any one of claims 11-21.

23. A vector comprising two of the nucleic acids of any one of claims 11-21, wherein the vector encodes the VL region and the VH region that together bind to SIGLEC15.

24. A pair of vectors, wherein each vector comprises one of the nucleic acids of any one of claims 11-21, wherein together the pair of vectors encodes the VL region and the VH region that together bind to SIGLEC15.

25. A cell comprising the vector of claim 22 or 23, or the pair of vectors of claim 24.

26. The cell of claim 25, wherein the cell is a CHO cell.

27. A cell comprising one or more of the nucleic acids of any one of claims 11-21.

28. A cell comprising two of the nucleic acids of any one of claims 11-21.

29. The cell of claim 28, wherein the two nucleic acids together encode the VL region and the VH region that together bind to SIGLEC15.

30. A method of producing an antibody or an antigen-binding fragment thereof, the method comprising
(c) culturing the cell of any one of claims 25-29 under conditions sufficient for the cell to produce the antibody or the antigen-binding fragment; and
(d) collecting the antibody or the antigen-binding fragment produced by the cell.

31. An antibody or antigen-binding fragment thereof that binds to SIGLEC15 comprising a heavy chain variable region (VH) comprising an amino acid sequence that is at least 90% identical to a selected VH sequence, and a light chain variable region (VL) comprising an amino acid sequence that is at least 90% identical to a selected VL sequence, wherein the selected VH sequence and the selected VL sequence are one of the following:
(1) the selected VH sequence is SEQ ID NO: 37, and the selected VL sequence is SEQ ID NO: 38;
(2) the selected VH sequence is SEQ ID NO: 39, and the selected VL sequence is SEQ ID NO: 40; and
(3) the selected VH sequence is SEQ ID NO: 41, and the selected VL sequence is SEQ ID NO: 42.

32. The antibody or antigen-binding fragment thereof of claim 31, wherein the VH comprises the sequence of SEQ ID NO: 37 and the VL comprises the sequence of SEQ ID NO: 38.

33. The antibody or antigen-binding fragment thereof of claim 31, wherein the VH comprises the sequence of SEQ ID NO: 39 and the VL comprises the sequence of SEQ ID NO: 40.

34. The antibody or antigen-binding fragment thereof of claim 31, wherein the VH comprises the sequence of SEQ ID NO: 41 and the VL comprises the sequence of SEQ ID NO: 42.

35. The antibody or antigen-binding fragment thereof of any one of claims 31-34, wherein the antibody or antigen-binding fragment thereof specifically binds to human, mouse, monkey, or dog SIGLEC15.

36. The antibody or antigen-binding fragment thereof of any one of claims 31-35, wherein the antibody or antigen-binding fragment thereof is a human antibody or antigen-binding fragment thereof, a single-chain variable fragment (scFv), a one-armed antibody, and or a multi-specific antibody (e.g., a bispecific antibody).

37. The antibody or antigen-binding fragment thereof of any one of claims 31-36, wherein the antibody or antigen-binding fragment is a human IgG1 antibody or antigen-binding fragment thereof or a human IgG4 antibody or antigen-binding fragment thereof.

38. An antibody or antigen-binding fragment thereof comprising the VH CDRs 1, 2, 3, and the VL CDRs 1, 2, 3 of the antibody or antigen-binding fragment thereof of any one of claims 1-10 and 31-37.

39. An antibody or antigen-binding fragment thereof that cross-competes with the antibody or antigen-binding fragment thereof of any one of claims 1-10 and 31-38.

40. The antibody or antigen-binding fragment thereof of any one of claims 1-10 and 31-39, wherein the antibody or antigen-binding fragment thereof comprises a fragment crystallizable region (Fc region).

41. The antibody or antigen-binding fragment thereof of claim 40, wherein the Fc region has reduced complement-dependent cytotoxicity (CDC) or antibody-dependent cellular cytotoxicity (ADCC).

42. A chimeric antigen receptor (CAR) comprising the antibody or antigen-binding fragment thereof of any one of claims 1-10 and 31-39.

43. An antibody-drug conjugate comprising the antibody or antigen-binding fragment thereof of any one of claims 1-10 and 31-41 covalently bound to a therapeutic agent.

44. The antibody drug conjugate of claim 43, wherein the therapeutic agent is a cytotoxic or cytostatic agent.

45. A method of treating a subject having cancer, the method comprising administering a therapeutically effective amount of a composition comprising the antibody or antigen-binding fragment thereof of any one of claims 1-10 and 31-41, or the antibody-drug conjugate of claim 43 or 44, to the subject.

46. The method of claim 45, wherein the subject has a solid tumor.

47. The method of claim 45, wherein the cancer is non-small-cell lung carcinoma (NSCLC), ovarian cancer, melanoma, colorectal cancer, breast cancer, colon adenocarcinoma, a hematological malignancy, head and neck cancer, gastrointestinal cancer, bladder cancer, or bone cancer.

48. The method of claim 45, wherein the cancer is Non-Hodgkin's lymphoma, lymphoma, leukemia, acute myeloid leukemia, or chronic lymphocytic leukemia.

49. The method of claim 45, wherein the subject is further treated with an effective amount of an anti-4-1BB antibody, an anti-OX40 antibody, anti-PD-1, anti-CTLA4, or an anti-PD-L1 antibody.

50. A method of decreasing the rate of tumor growth, the method comprising contacting a tumor cell with an effective amount of a composition comprising an antibody or antigen-binding fragment thereof of any one of claims 1-10 and 31-41, or the antibody-drug conjugate of claim 43 or 44.

51. A method of killing a tumor cell, the method comprising contacting a tumor cell with an effective amount of a composition comprising the antibody or antigen-binding fragment thereof of any one of claims 1-10 and 31-41, or the antibody-drug conjugate of claim 43 or 44.

52. A method of increasing immune response in a subject, the method comprising administering to the subject an effective amount of a composition comprising the antibody or antigen-binding fragment thereof of any one of claims 1-10 and 31-41.

53. A method of treating a subject having a bone disease, the method comprising administering a therapeutically effective amount of a composition comprising the antibody or antigen-binding fragment thereof of any one of claims 1-10 and 31-41 to the subject.

54. The method of claim 53, wherein the bone disease is osteoporosis.

55. A pharmaceutical composition comprising the antibody or antigen-binding fragment thereof of any one of claims 1-10 and 31-41, and a pharmaceutically acceptable carrier.

56. A pharmaceutical composition comprising the antibody drug conjugate of claim 43 or 44, and a pharmaceutically acceptable carrier.


SEQUENCE LISTING (78 sequences)

SEQ ID NO: 1 - protein, 5 aa, synthetic construct
NYEMN

SEQ ID NO: 2 - protein, 17 aa, synthetic construct
YISNSGSTIYYADSVKG

SEQ ID NO: 3 - protein, 13 aa, synthetic construct
EDYDSSGYYWFDP

SEQ ID NO: 4 - protein, 11 aa, synthetic construct
RASQGISSWLA

SEQ ID NO: 5 - protein, 7 aa, synthetic construct
AASSLQS

SEQ ID NO: 6 - protein, 9 aa, synthetic construct
QQTNSFPLT

SEQ ID NO: 7 - protein, 5 aa, synthetic construct
NYGFS

SEQ ID NO: 8 - protein, 17 aa, synthetic construct
WINTYNDNTNYAQKFQG

SEQ ID NO: 9 - protein, 14 aa, synthetic construct
DGLWFGELPHYFDY

SEQ ID NO: 10 - protein, 16 aa, synthetic construct
RSSQSLVNSDGETFLS

SEQ ID NO: 11 - protein, 7 aa, synthetic construct
KISNRFS

SEQ ID NO: 12 - protein, 9 aa, synthetic construct
MQATQFPHT

SEQ ID NO: 13 - protein, 5 aa, synthetic construct
GYFWS

SEQ ID NO: 14 - protein, 16 aa, synthetic construct
EINHSGSTNYNPSLKS

SEQ ID NO: 15 - protein, 13 aa, synthetic construct
EGDSSSWGNWFDP

SEQ ID NO: 16 - protein, 12 aa, synthetic construct
RASQSVSSSYLA

SEQ ID NO: 17 - protein, 7 aa, synthetic construct
GASSRAT

SEQ ID NO: 18 - protein, 9 aa, synthetic construct
QQFGNSPLT

SEQ ID NO: 19 - protein, 7 aa, synthetic construct
GFTFSNY

SEQ ID NO: 20 - protein, 6 aa, synthetic construct
SNSGST

SEQ ID NO: 21 - protein, 13 aa, synthetic construct
EDYDSSGYYWFDP

SEQ ID NO: 22 - protein, 11 aa, synthetic construct
RASQGISSWLA

SEQ ID NO: 23 - protein, 7 aa, synthetic construct
AASSLQS

SEQ ID NO: 24 - protein, 9 aa, synthetic construct
QQTNSFPLT

SEQ ID NO: 25 - protein, 7 aa, synthetic construct
GYTFTNY

SEQ ID NO: 26 - protein, 6 aa, synthetic construct
NTYNDN

SEQ ID NO: 27 - protein, 14 aa, synthetic construct
DGLWFGELPHYFDY

SEQ ID NO: 28 - protein, 16 aa, synthetic construct
RSSQSLVNSDGETFLS

SEQ ID NO: 29 - protein, 7 aa, synthetic construct
KISNRFS

SEQ ID NO: 30 - protein, 9 aa, synthetic construct
MQATQFPHT

SEQ ID NO: 31 - protein, 7 aa, synthetic construct
GGSFSGY

SEQ ID NO: 32 - protein, 5 aa, synthetic construct
NHSGS

SEQ ID NO: 33 - protein, 13 aa, synthetic construct
EGDSSSWGNWFDP

SEQ ID NO: 34 - protein, 12 aa, synthetic construct
RASQSVSSSYLA

SEQ ID NO: 35 - protein, 7 aa, synthetic construct
GASSRAT

SEQ ID NO: 36 - protein, 9 aa, synthetic construct
QQFGNSPLT

SEQ ID NO: 37 - protein, 122 aa, synthetic construct
EVQLVQSGGGLVQPGGSLRLSCAASGFTFSNYEMNWVRQAPGKGLEWISYISNSGSTIYYADSVKGRFTISRDDAKNSLYLQMNSLRAEDTAVYYCAREDYDSSGYYWFDPWGQGTPVTVSS

SEQ ID NO: 38 - protein, 107 aa, synthetic construct
DIQLTQSPSSVSASVGDRVTITCRASQGISSWLAWYQQKPGKAPKLLIYAASSLQSGVPSRFSGSGSGTDFTLTINSLQPEDFATYYCQQTNSFPLTFGGGTKVEIK

SEQ ID NO: 39 - protein, 123 aa, synthetic construct
QVQLVQSGAEVKKPGATVKVSCKASGYTFTNYGFSWVRQAPGQGLEWMGWINTYNDNTNYAQKFQGRVTMTTDTSTSTAYMELRSLRSDDTAVYYCARDGLWFGELPHYFDYWGQGTLVTVSS

SEQ ID NO: 40 - protein, 112 aa, synthetic construct
DIQLTQSPLSSPVTLGQPASISCRSSQSLVNSDGETFLSWLQQRPGQPPRLLIYKISNRFSGVPDRFSGSGAGTDFTLKISRVEAEDVGVYYCMQATQFPHTFGQGTKLEIK

SEQ ID NO: 41 - protein, 121 aa, synthetic construct
QVQLQQWGAGLLKPSETLSLTCAVYGGSFSGYFWSWIRQPPGKGLEWIGEINHSGSTNYNPSLKSRVTLSVDTSKNQFSLKLSSVTAADTAVYYCAREGDSSSWGNWFDPWGQGTLVTVSS

SEQ ID NO: 42 - protein, 108 aa, synthetic construct
EIVLTQSPGTLSLSPGERATLSCRASQSVSSSYLAWYQQKPGQAPRFLIYGASSRATGIPDRFSGSGSGTDFTLTISRLEPEDFAVYYCQQFGNSPLTFGGGTKVEIK

SEQ ID NO: 43 - protein, 453 aa, synthetic construct
QVQLVQSGAEVKKPGATVKVSCKASGYTFTNYGFSWVRQAPGQGLEWMGWINTYNDNTNYAQKFQGRVTMTTDTSTSTAYMELRSLRSDDTAVYYCARDGLWFGELPHYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 45 - protein, 328 aa, Homo sapiens
MEKSIWLLACLAWVLPTGSFVRTKIDTTENLLNTEVHSSPAQRWSMQVPPEVSAEAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVRLHVTAAPRIVNISVLPSPAHAFRALCTAEGEPPPALAWSGPALGNSLAAVRSPREGHGHLVTAELPALTHDGRYTCTAANSLGRSEASVYLFRFHGASGASTVALLLGALGFKALLLLGVLAARAARRRPEHLDTPDTPPRSQAQESNYENLSQMNPRSPPATMCSP

SEQ ID NO: 46 - protein, 342 aa, Mus musculus
MEGSLQLLACLACVLQMGSLVKTRRDASGDLLNTEAHSAPAQRWSMQVPAEVNAEAGDAAVLPCTFTHPHRHYDGPLTAIWRSGEPYAGPQVFRCTAAPGSELCQTALSLHGRFRLLGNPRRNDLSLRVERLALADSGRYFCRVEFTGDAHDRYESRHGVRLRVTAAAPRIVNISVLPGPAHAFRALCTAEGEPPPALAWSGPAPGNSSAALQGQGHGYQVTAELPALTRDGRYTCTAANSLGRAEASVYLFRFHGAPGTSTLALLLGALGLKALLLLGILGARATRRRLDHLVPQDTPPRSQAQESNYENLSQMSPPGHQLPRVCCEELLSHHHLVIHHEK

SEQ ID NO: 47 - protein, 328 aa, Macaca fascicularis
MESSIRLLACLACVLPTGSFVRTKIDTTENLLNTEVHSSPAQRWSMQVPAEVSAAAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVRLHVTAAPRIINISVLPGPAHAFRALCTAEGEPPPALAWSGPALGNGSAAVPSSGQGHGHLVTAELPALNHDGRYTCTAANSLGRSEASVYLFRFHGASGASTVALLLGALGLKALLLLGVLAAGVARRRPEHLNTPDTPPRSQAQESNYENLSQMNPRSPPAAMCSP

SEQ ID NO: 48 - protein, 353 aa, Canis lupus
MEWFIRLLACLVGILPMGSFVRTKRDTTGNLLNTEVHSAPAQRWSMQVPAEVSAAAGEAAVLPCTFTHPHRHYDGPLTAIWRAGEVYAGPQVFRCAAARGSELCQTALSLHGRFRLLGNPRRNDLSLRVERLALADDGRYFCRVEFAGDVHDRYESRHGVRLRVTAAPRIVSLSVLPGPAHAFRALCTAEGEPPPVLAWSGPALGNRSAAASGPGQGHGHQVTAELPALAHDGRYTCTASNSLGRAEASVYLFRFRGAPGAPADYKDDDDKDYKDDDDKDYKDDDDKLALPLGALGLKALLLLGVLAARAAARRRREHPVAQDTPPWPQAQESNYENLSQMSPRDPAAATCLP

SEQ ID NO: 49 - protein, 1622 aa, Homo sapiens
SWGVSSPQDVQGVKGSCLLIPCIFSFPADVEVPDGITAIWYYDYSGQRQVVSHSADPKLVEARFRGRTEFMGNPEHRVCNLLLKDLQPEDSGSYNFRFEISEVNRWSDVKGTLVTVTEEPRVPTIASPVELLEGTEVDFNCSTPYVCLQEQVRLQWQGQDPARSVTFNSQKFEPTGVGHLETLHMAMSWQDHGRILRCQLSVANHRAQSEIHLQVKYAPKGVKILLSPSGRNILPGELVTLTCQVNSSYPAVSSIKWLKDGVRLQTKTGVLHLPQAAWSDAGVYTCQAENGVGSLVSPPISLHIFMAEVQVSPAGPILENQTVTLVCNTPNEAPSDLRYSWYKNHVLLEDAHSHTLRLHLATRADTGFYFCEVQNVHGSERSGPVSVVVNHPPLTPVLTAFLETQAGLVGILHCSVVSEPLATLVLSHGGHILASTSGDSDHSPRFSGTSGPNSLRLEIRDLEETDSGEYKCSATNSLGNATSTLDFHANAARLLISPAAEVVEGQAVTLSCRSGLSPTPDARFSWYLNGALLHEGPGSSLLLPAASSTDAGSYHCRARDGHSASGPSSPAVLTVLYPPRQPTFTTRLDLDAAGAGAGRRGLLLCRVDSDPPARLQLLHKDRVVATSLPSGGGCSTCGGCSPRMKVTKAPNLLRVEIHNPLLEEEGLYLCEASNALGNASTSATFNGQATVLAIAPSHTLQEGTEANLTCNVSREAAGSPANFSWFRNGVLWAQGPLETVTLLPVARTDAALYACRILTEAGAQLSTPVLLSVLYPPDRPKLSALLDMGQGHMALFICTVDSRPLALLALFHGEHLLATSLGPQVPSHGRFQAKAEANSLKLEVRELGLGDSGSYRCEATNVLGSSNTSLFFQVRGAWVQVSPSPELQEGQAVVLSCQVHTGVPEGTSYRWYRDGQPLQESTSATLRFAAITLTQAGAYHCQAQAPGSATTSLAAPISLHVSYAPRHVTLTTLMDTGPGRLGLLLCRVDSDPPAQLRLLHGDRLVASTLQGVGGPEGSSPRLHVAVAPNTLRLEIHGAMLEDEGVYICEASNTLGQASASADFDAQAVNVQVWPGATVREGQLVNLTCLVWTTHPAQLTYTWYQDGQQRLDAHSIPLPNVTVRDATSYRCGVGPPGRAPRLSRPITLDVLYAPRNLRLTYLLESHGGQLALVLCTVDSRPPAQLALSHAGRLLASSTAASVPNTLRLELRGPQPRDEGFYSCSARSPLGQANTSLELRLEGVRVILAPEAAVPEGAPITVTCADPAAHAPTLYTWYHNGRWLQEGPAASLSFLVATRAHAGAYSCQAQDAQGTRSSRPAALQVLYAPQDAVLSSFRDSRARSMAVIQCTVDSEPPAELALSHDGKVLATSSGVHSLASGTGHVQVARNALRLQVQDVPAGDDTYVCTAQNLLGSISTIGRLQVEGARVVAEPGLDVPEGAALNLSCRLLGGPGPVGNSTFAWFWNDRRLHAEPVPTLAFTHVARAQAGMYHCLAELPTGAAASAPVMLRVLYPPKTPTMMVFVEPEGGLRGILDCRVDSEPLASLTLHLGSRLVASSQPQGAPAEPHIHVLASPNALRVDIEALRPSDQGEYICSASNVLGSASTSTYFGVRALHRLHQFQQ

SEQ ID NO: 50 - protein, 668 aa, Homo sapiens
DSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRR

SEQ ID NO: 51 - protein, 497 aa, Homo sapiens
GHWGAWMPSSISAFEGTCVSIPCRFDFPDELRPAVVHGVWYFNSPYPKNYPPVVFKSRTQVVHESFQGRSRLLGDLGLRNCTLLLSNVSPELGGKYYFRGDLGGYNQYTFSEHSVLDIVNTPNIVVPPEVVAGTEVEVSCMVPDNCPELRPELSWLGHEGLGEPAVLGRLREDEGTWVQVSLLHFVPTREANGHRLGCQASFPNTTLQFEGYASMDVKYPPVIVEMNSSVEAIEGSHVSLLCGADSNPPPLLTWMRDGTVLREAVAESLLLELEEVTPAEDGVYACLAENAYGQDNRTVGLSVMYAPWKPTVNGTMVAVEGETVSILCSTQSNPDPILTIFKEKQILSTVIYESELQLELPAVSPEDDGEYWCVAENQYGQRATAFNLSVEFAPVLLLESHCAAARDTVQCLCVVKSNPEPSVAFELPSRNVTVNESEREFVYSERSGLVLTSILTLRGQAQAPPRVICTARNLYGAKSLELPFQGAHRLMWAKIGP

SEQ ID NO: 52 - protein, 425 aa, Homo sapiens
EKPVYELQVQKSVTVQEGLCVLVPCSFSYPWRSWYSSPPLYVYWFRDGEIPYYAEVVATNNPDRRVKPETQGRFRLLGDVQKKNCSLSIGDARMEDTGSYFFRVERGRDVKYSYQQNKLNLEVTALIEKPDIHFLEPLESGRPTRLSCSLPGSCEAGPPLTFSWTGNALSPLDPETTRSSELTLTPRPEDHGTNLTCQMKRQGAQVTTERTVQLNVSYAPQTITIFRNGIALEILQNTSYLPVLEGQALRLLCDAPSNPPAHLSWFQGSPALNATPISNTGILELRRVRSAEEGGFTCRAQHPLGFLQIFLNLSVYSLPQLLGPSCSWEAEGLHCRCSFRARPAPSLCWRLEEKPLEGNSSQGSFKVNSSSAGPWANSSLILHGGLSSDLKVSCKAWNIYGSQSGSVLLLQGRSNLGTGVVPAAL

SEQ ID NO: 53 - protein, 321 aa, Homo sapiens
QERRFQLEGPESLTVQEGLCVLVPCRLPTTLPASYYGYGYWFLEGADVPVATNDPDEEVQEETRGRFHLLWDPRRKNCSLSIRDARRRDNAAYFFRLKSKWMKYGYTSSKLSVRVMALTHRPNISIPGTLESGHPSNLTCSVPWVCEQGTPPIFSWMSAAPTSLGPRTTQSSVLTITPRPQDHSTNLTCQVTFPGAGVTMERTIQLNVSYAPQKVAISIFQGNSAAFKILQNTSSLPVLEGQALRLLCDADGNPPAHLSWFQGFPALNATPISNTGVLELPQVGSAEEGDFTCRAQHPLGSLQISLSLFVHWKPEGRAGGV

SEQ ID NO: 54 - protein, 335 aa, Homo sapiens
QKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFRMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGVLL

SEQ ID NO: 55 - protein, 347 aa, Homo sapiens [sequence omitted]

SEQ ID NO: 56 - protein, 331 aa, Homo sapiens
QTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQG

SEQ ID NO: 57 - protein, 534 aa, Homo sapiens
MDGRFWIRVQESVMVPEGLCISVPCSFSYPRQDWTGSTPAYGYWFKAVTETTKGAPVATNHQSREVEMSTRGRFQLTGDPAKGNCSLVIRDAQMQDESQYFFRVERGSYVRYNFMNDGFFLKVTALTQKPDVYIPETLEPGQPVTVICVFNWAFEECPPPSFSWTGAALSSQGTKPTTSHFSVLSFTPRPQDHNTDLTCHVDFSRKGVSAQRTVRLRVAYAPRDLVISISRDNTPALEPQPQGNVPYLEAQKGQFLRLLCAADSQPPATLSWVLQNRVLSSSHPWGPRPLGLELPGVKAGDSGRYTCRAENRLGSQQRALDLSVQYPPENLRVMVSQANRTVLENLGNGTSLPVLEGQSLCLVCVTHSSPPARLSWTQRGQVLSPSQPSDPGVLELPRVQVEHEGEFTCHARHPLGSQHVSLSLSVHYSPKLLGPSCSWEAEGLHCSCSSQASPAPSLRWWLGEELLEGNSSQDSFEVTPSSAGPWANSSLSLHGGLSSGLRLRCEAWNVHGAQSGSILQLPDKKGLISTAFSN

SEQ ID NO: 58 - protein, 458 aa, Homo sapiens
QLTGDPGKGSCSLVIRDAQREDEAWYFFRVERGSRVRHSFLSNAFFLKVTALTKKPDVYIPETLEPGQPVTVICVFNWAFKKCPAPSFSWTGAALSPRRTRPSTSHFSVLSFTPSPQDHDTDLTCHVDFSRKGVSAQRTVRLRVAYAPKDLIISISHDNTSALELQGNVIYLEVQKGQFLRLLCAADSQPPATLSWVLQDRVLSSSHPWGPRTLGLELRGVRAGDSGRYTCRAENRLGSQQQALDLSVQYPPENLRVMVSQANRTVLENLGNGTSLPVLEGQSLRLVCVTHSSPPARLSWTRWGQTVGPSQPSDPGVLELPPIQMEHEGEFTCHAQHPLGSQHVSLSLSVHYPPQLLGPSCSWEAEGLHCSCSSQASPAPSLRWWLGEELLEGNSSQGSFEVTPSSAGPWANSSLSLHGGLSSGLRLRCKAWNVHGAQSGSVFQLLPGKLEHGGGLGL

SEQ ID NO: 59 - protein, 463 aa, Homo sapiens
KEQKDYLLTMQKSVTVQEGLCVSVLCSFSYPQNGWTASDPVHGYWFRAGDHVSRNIPVATNNPARAVQEETRDRFHLLGDPQNKDCTLSIRDTRESDAGTYVFCVERGNMKWNYKYDQLSVNVTASQDLLSRYRLEVPESVTVQEGLCVSVPCSVLYPHYNWTASSPVYGSWFKEGADIPWDIPVATNTPSGKVQEDTHGRFLLLGDPQTNNCSLSIRDARKGDSGKYYFQVERGSRKWNYIYDKLSVHVTALTHMPTFSIPGTLESGHPRNLTCSVPWACEQGTPPTITWMGASVSSLDPTITRSSMLSLIPQPQDHGTSLTCQVTLPGAGVTMTRAVRLNISYPPQNLTMTVFQGDGTASTTLRNGSALSVLEGQSLHLVCAVDSNPPARLSWTWGSLTLSPSQSSNLGVLELPRVHVKDEGEFTCRAQNPLGSQHISLSLSLQNEYTGKMRPISGVTLGA

SEQ ID NO: 60 - protein, 342 aa, Homo sapiens
EKPVYELQVQKSVTVQEGLCVLVPCSFSYPWRSWYSSPPLYVYWFRDGEIPYYAEVVATNNPDRRVKPETQGRFRLLGDVQKKNCSLSIGDARMEDTGSYFFRVERGRDVKYSYQQNKLNLEVTALIEKPDIHFLEPLESGRPTRLSCSLPGSCEAGPPLTFSWTGNALSPLDPETTRSSELTLTPRPEDHGTNLTCQVKRQGAQVTTERTVQLNVSYAPQNLAISIFFRNGTGTALRILSNGMSVPIQEGQSLFLACTVDSNPPASLSWFREGKALNPSQTSMSGTLELPNIGAREGGEFTCRVQHPLGSQHLSFILSVQRSSSSCICVTEKQQGSWPLVL

SEQ ID NO: 61 - protein, 418 aa, Homo sapiens
NKDPSYSLQVQRQVPVPEGLCVIVSCNLSYPRDGWDESTAAYGYWFKGWTSPKTGAPVATNNQSREVEMSTRDRFQLTGDPGKGSCSLVIRDAQREDEAWYFFRVERGSRVRHSFVNNLFFLKVTALTQKPDVYIPETLEPGQPVTVICVFNWAFKKCPAPSFSWTGAALSPRRTRPSTSHFSVLSFTPSPQDHDTDLTCHVDFSRKGVSAQRTVRLRVASLELQGNVIYLEVQKGQFLRLLCAADSQPPATLSWVLQDRVLSSSHPWGPRTLGLELRGVRAGDSGRYTCRAENRLGSQQRALDLSVQYPPENLRVMVSQANRTVLENLRNGTSLRVLEGQSLRLVCVTHSSPPARLSWTRWGQTVGPSQPSDPGVLELPRVQMEHEGEFTCHARHPLGSQRVSLSFSVHCKSGPMTG

SEQ ID NO: 62 - protein, 327 aa, Macaca fascicularis
MESSIRLLACLACVLPTGSFVRTKIDTTENLLNTEVHSSPVQRWSMQVPAEVSAAAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVRLHVTAAPRIINISVLPGPAHAFRALCTTEGEPPPALAWSGPALGNGSAAVPSSGQGHGHLVTAELPALNHDGRYTCTAANSLGRSEASVYLFRFHGASASTVALLLGALGLKALLLLGVLAAGVARHRPEHLNTPDTPPRSQAQESNYENLSQMNPRSPPAAMCSP

SEQ ID NO: 63 - protein, 452 aa, synthetic construct
QVQLQQPGAELVKPGASVKMSCKASGYTFTSYWITWVIQRPGQGLEWIGDIYCGSDTMHYNEKFKNKATLTVDTSSSTAYMQLSSLTSEDSAVYYCARWWDYGSSYDYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 214 aa, synthetic construct
DIKMTQSPSSMYASLGERVTITCKASQDINSYLSWFQQKPGKSPKTLIYRANRLVDGVPSRFSGSGSGQDYSLTISSLEYEDMGIYYCLQYDEFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 65 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 343 aa, synthetic construct
MEGSLQLLACLACVLQMGSLVKTRRDASGDLLNTEVHSSPAQRWSMQVPPEVSAEAGDAAVLPCTFTHPHRHYDGPLTAIWRAGEPYAGPQVFRCAAARGSELCQTALSLHGRFRLLGNPRRNDLSLRVERLALADDRRYFCRVEFAGDVHDRYESRHGVRLHVTAAPRIVNISVLPSPAHAFRALCTAEGEPPPALAWSGPALGNSLAAVRSPREGHGHLVTAELPALTHDGRYTCTAANSLGRSEASVYLFRFHGAPGTSTLALLLGALGLKALLLLGILGARATRRRLDHLVPQDTPPRSQAQESNYENLSQMSPPGHQLPRVCCEELLSHHHLVIHHEK

SEQ ID NO: 68 - protein, 257 aa, synthetic construct
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSLQVLTLFLALTSALLLALIFITLLFSVLKWIRKKFPHIFKQPFKKTTGAAQEEDACSCRCPQEEEGGGGGYEL

SEQ ID NO: 69 - protein, 270 aa, synthetic construct
MYVWVQQPTALLLLGLTLGVTARRLNCVKHTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSELPSPPTLVTPEGPAFAVLLGLGLGLLAPLTVLLALYLLRKAWRLPNTPKPCWGNSFRTPIQEEHTDAHFTLAKI

SEQ ID NO: 70 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 71 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLYITREPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 76 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 77 - protein, 327 aa, synthetic construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 78 - protein, 219 aa, synthetic construct
DIQLTQSPLSSPVTLGQPASISCRSSQSLVNSDGETFLSWLQQRPGQPPRLLIYKISNRFSGVPDRFSGSGAGTDFTLKISRVEAEDVGVYYCMQATQFPHTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC